(19) United States
(12) Patent Application Publication
(10) Pub. No.: US 2025/0066767 A1
(43) Pub. Date: Feb. 27, 2025

(54) COMPOSITIONS AND METHODS FOR MAKING GUIDE NUCLEIC ACIDS

(71) Applicant: ARC BIO, LLC, Cambridge, MA (US)

(72) Inventors: Morten RASMUSSEN, Cambridge, MA (US); Stephane B. GOURGUECHON, Cambridge, MA (US)

(21) Appl. No.: 18/599,123

(22) Filed: Mar. 7, 2024

Related U.S. Application Data
(63) Continuation of application No. 17/057,390, filed on Nov. 20, 2020, now abandoned.
(60) Provisional application No. 62/682,140, filed on Jun. 7, 2018.

(51) Int. Cl. C12N 15/10; C12N 9/22; C12N 15/11

(52) U.S. Cl. CPC C12N 15/1068; C12N 9/22; C12N 15/11; C12N 2310/20; C12N 2800/80

(57) ABSTRACT

Provided are compositions and methods of making a guide nucleic acids (gNAs), methods of using gNAs. and ligation free methods of preparing libraries of nucleic acids for downstream applications such as high-throughput sequencing.

### RELATED APPLICATIONS

This application claims the benefit of priority to U.S. provisional patent application Ser. No. 62/682,140 filed on Jun. 7, 2018, the contents of which are incorporated by reference in their entirety.

### STATEMENT OF GOVERNMENT INTEREST

This invention was made with government support under 2017_DN_BX_0140 awarded by the National Institute of Justice. The government has certain rights in the invention.

### INCORPORATION OF SEQUENCE LISTING

The instant application contains a Sequence Listing which has been submitted in ASCII format via EFS-Web and is hereby incorporated by reference in its entirety. Said ASCII copy, created on Jun. 6, 2019 is named ARCB-01201WO_ST25.txt and is 3 kilobytes in size.

### BACKGROUND

Conventional techniques of preparing libraries of nucleic acids for high throughput sequencing use ligation to introduce adapters onto the 5′ and 3′ ends of the nucleic acids. However, these techniques may not be suitable for small and/or highly degraded samples. There thus exists a need in the art for additional, ligation-free methods of library preparation. The disclosure provides ligation-free methods of library preparation suitable for small and/or highly degraded samples.

In addition, many RNA polymerases can add untemplated nucleotides to the 3′ ends of in vitro transcribed RNAs. These additional untemplated nucleotides may negatively affect the function of in vitro transcribed RNAs. Thus there exists a need in the art to generate in vitro transcribed RNAs that do not contain untemplated 3′ nucleotides. The invention provides compositions and methods for generating in vitro transcribed RNAs that do not contain untemplated 3′ nucleotides.

### SUMMARY

The disclosure provides methods of preparing a library of nucleic acids, comprising: (a) providing a sample of nucleic acids comprising at least one sequence of interest; (b) contacting the sample of nucleic acids, a plurality of first polymerase chain reaction (PCR) primers and a polymerase under conditions that allow PCR to occur, thereby generating a plurality of first single-sided PCR products; (c) contacting the plurality of first single-sided PCR products with a terminal transferase under conditions sufficient to transfer dNTPs to the 3′ ends of the plurality of first single-sided PCR products, thereby generating a plurality of PCR products comprising 3′ tails; and (d) contacting the plurality of PCR products comprising 3′ tails, a plurality of second PCR primers and a polymerase under conditions that allow PCR to occur; thereby generating a library of nucleic acids with adapters at the 5′ and 3′ ends.

In some embodiments of the methods of the disclosure, the methods comprise (e) contacting the plurality of PCR products from (d) with a plurality of first indexing primers, a plurality of second indexing primers, and a polymerase under conditions that allow PCR to occur.

In some embodiments of the methods of the disclosure, the methods comprise contacting the sample of nucleic acids with an enzyme prior to step (b) under conditions that allow for blunting of overhangs in the sample of nucleic acids, thereby generating a blunt-ended sample of nucleic acids.

In some embodiments of the methods of the disclosure, the methods comprise contacting the blunt-ended sample of nucleic acids with an enzyme under conditions that allow for the addition of dideoxynucleotides (ddNTPs) to the to the 3′ ends of the blunt ended nucleic acids in the sample, wherein contacting the blunt-ended sample of nucleic acids with an enzyme occurs prior to step (b).

The disclosure provides methods of preparing a library of nucleic acids, comprising: (a) providing a sample of nucleic acids comprising at least one sequence of interest; (b) contacting the sample of nucleic acids with a terminal transferase under conditions sufficient to transfer NTPs to the 3′ end of the nucleic acids, thereby generating a plurality of nucleic acids comprising 3′ tails; (c) contacting the plurality of nucleic acids comprising 3′ tails with a plurality of first adapters and a reverse transcriptase under conditions sufficient for first strand complementary DNA (cDNA) synthesis to occur, thereby generating a plurality of cDNAs, wherein the plurality of cDNAs comprise 3′ polyC sequences; and (d) contacting the plurality of cDNAs with a second adapter under conditions sufficient to allow generation of double stranded DNA from the plurality of cDNAs to generate a plurality of double stranded DNAs, thereby preparing a library of nucleic acids with adapters at the 5′ and 3′ ends.

In some embodiments, the methods comprise (a) providing a plurality of guide nucleic acid (gNA)-CRISPR/Cas system protein complexes, wherein the gNAs are configured to hybridize to at least one sequence targeted for depletion; (b) mixing the library of nucleic acids with the plurality of gNA-CRISPR/Cas system protein complexes, wherein at least a portion of the gNA-CRISPR/Cas system protein complexes hybridize to the at least one sequence targeted for depletion; and (d) incubating the mixture to cleave the at least one sequence targeted for depletion.

The disclosure provides in vitro methods of making guide ribonucleic acids (gRNAs), overcoming challenges associated with RNA polymerases adding untemplated nucleotides to the 3′ ends of the gRNAs during transcription. In some embodiments of the methods of the disclosure, the method comprises separating in vitro transcribed RNAs such as gRNAs based on size. In some embodiments of the methods of the disclosure, the method comprises adding 3′ primer binding site to the in vitro transcribed RNA. In some embodiments, this primer binding site is hybridized to a DNA oligonucleotide, and the resulting DNA: RNA heteroduplex cleaved with RNase H or a restriction enzyme.

### BRIEF DESCRIPTION OF THE DRAWINGS

FIG. 1 is a diagram of Cas9 system-compatible and Cpf1 system-compatible gRNAs generated by in vitro transcription using T7 RNA polymerase, oriented with the 5′ end of the polynucleotide to the left.

FIG. 2 is a diagram showing methods for removing untemplated 3′ nucleotides from an in vitro transcribed RNA such as a Cpf1 gRNA by annealing a DNA oligo to a primer binding site and then cutting the DNA-RNA heteroduplex with a restriction enzyme or RNAse H.

FIG. 3 illustrates an exemplary scheme for a guide nucleic acid library from a DNA source that has been cut with either MseI or MluCI and treated with mung bean nuclease to degrade single stranded overhangs.

FIG. 4A and FIG. 4B illustrate an exemplary scheme for a guide nucleic acid library from a DNA source in which adenosines have been replaced with inosines.

FIG. 5A and FIG. 5B illustrate an exemplary scheme for a guide nucleic acid library from a DNA source in which thymidines have been replaced with uracils.

FIG. 6 illustrates an exemplary scheme for a guide nucleic acid library from a DNA source that has been randomly fragmented with a non-specific nickase and T7 endonuclease I (fragmentase).

FIG. 7A and FIG. 7B illustrate an exemplary scheme for a guide nucleic acid library from a DNA source that has been randomly sheared and methylated.

FIG. 8A, FIG. 8B and FIG. 8C illustrate an exemplary scheme for a guide nucleic acid library from a randomly sheared DNA source.

FIG. 9A and FIG. 9B illustrate an exemplary scheme for a guide nucleic acid library from a randomly sheared DNA source using the ligation of a circular adapter.

FIG. 10A, FIG. 10B, FIG. 10C and FIG. 10D illustrate an exemplary scheme for a guide nucleic acid library from a randomly sheared DNA source that has been blunt end repaired.

FIG. 11A, FIG. 11B and FIG. 11C illustrate an exemplary scheme for a guide nucleic acid library from a randomly sheared DNA source that has been blunt end repaired.

FIG. 12 illustrates an exemplary scheme for a guide nucleic acid library from a randomly sheared DNA source that has been circularized.

FIG. 13 illustrates an exemplary scheme for designing collections of guide nucleic acids.

FIG. 14 illustrates an exemplary scheme for designing collections of guide nucleic acids.

FIG. 15 illustrates an exemplary scheme for depleting, partitioning, or capturing targeted nucleic acids.

FIG. 16 illustrates an exemplary schematic of a strand-switching method.

FIG. 17 illustrates an exemplary scheme for the library generation and enrichment in a single workflow.

FIG. 18 is an Agilent High Sensitivity D1000 gel illustrating the DNA fragment distribution of ligation free sequencing libraries following indexing and purification, and an A-tailing negative control sample. At top, the wells from left to right are: EL1 (ladder), A1 (iPCR1-Pur-Neg, “Negative” sample), B1 (iPCR1-Pur-Test, “Test” Sample), C1 (iPCR1-Pur-Pos, “Positive” Sample) and D1 (PCR10-Atail-Neg, the A-tailing Negative Control).

FIG. 19 is a plot illustrating the size (x-axis, in base pairs [bp]) and intensity (y-axis, normalized fluorescence units, abbreviated FU) of the ladder (EL1). Lines and brackets indicate regions used to calculate the parameters disclosed in Table 15.

FIG. 20A is a plot illustrating the size (x-axis, in bp) and intensity (y-axis, FU) of the Negative sample (iPCR1-Pur-Neg) following indexing and purification. Lines and brackets indicate regions used to calculate the parameters disclosed in Table 16.

FIG. 20B is a plot illustrating the size (x-axis, in bp) and intensity (y-axis, FU) of the Negative sample (iPCR1-Pur-Neg) following indexing and purification. Lines and brackets indicate regions used to calculate the parameters disclosed in Table 17.

FIG. 21A is a plot illustrating the size (x-axis, in bp) and intensity (y-axis, FU) of the Test sample (iPCR1-Pur-Test) following indexing and purification. Lines and brackets indicate regions used to calculate the parameters disclosed in Table 18.

FIG. 21B is a plot illustrating the size (x-axis, in bp) and intensity (y-axis, FU) of the Test sample (iPCR1-Pur-Test) following indexing and purification. Lines and brackets indicate regions used to calculate the parameters disclosed in Table 19. Dark lines indicate from 100-1000 bp, light lines indicate from 265-1000 bp.

FIG. 22A is a plot illustrating the size (x-axis, in bp) and intensity (y-axis, FU) of the Positive sample (iPCR1-Pur-Pos) following indexing and purification. Lines and brackets indicate regions used to calculate the parameters disclosed in Table 20.

FIG. 22B is a plot illustrating the size (x-axis, in bp) and intensity (y-axis, FU) of the Positive sample (iPCR1-Pur-Pos) following indexing and purification. Lines and brackets indicate regions used to calculate the parameters disclosed in Table 21. Dark lines indicate from 100-1000 bp, light lines indicate from 265-1000 bp.

FIG. 23A is a plot illustrating the size (x-axis, in bp) and intensity (y-axis, FU) of the A-tailing negative sample (PCR10-Atail-Neg). Lines and brackets indicate regions used to calculate the parameters disclosed in Table 22.

FIG. 23B is a plot illustrating the size (x-axis, in bp) and intensity (y-axis, FU) of the A-tailing negative sample (PCR10-Atail-Neg). Lines and brackets indicate regions used to calculate the parameters disclosed in Table 23. Dark lines indicate from 100-1000 bp, light lines indicate from 265-1000 bp.

FIG. 24A is an Agilent High Sensitivity D1000 gel illustrating a profile comparison of A1 (iPCR1-Pur-Neg, “Negative” sample), B1 (iPCR1-Pur-Test, “Test” Sample), C1 (iPCR1-Pur-Pos, “Positive” Sample).

FIG. 24B is a plot illustrating a profile comparison of A1 (iPCR1-Pur-Neg, “Negative” sample, green), B1 (iPCR1-Pur-Test, “Test” Sample, orange), C1 (iPCR1-Pur-Pos, “Positive” Sample, blue). Size in bp is plotted on the x-axis, sample intensity (Normalized FU) is plotted on the y-axis.

FIG. 25 is a plot illustrating the distribution of fragment sizes (read lengths) from high throughput sequencing of the Test and Positive samples.

FIG. 26A is a plot illustrating the sequence counts for the Positive and Test samples. Duplicate read counts are an estimate only.

FIG. 26B is a plot illustrating the percentage of Unique and Duplicate Reads for the Positive and Test samples. Duplicate read counts are an estimate only.

FIG. 27 is a plot illustrating the mean sequence quality value across each base position in the read. The Test sample is shown in dark gray, the Positive sample is shown in light gray.

FIG. 28 is a plot illustrating the number of reads with average quality scores. This shows if a subset of reads have poor quality. The Positive sample is the top line, the Test sample is the lower line.

FIG. 29 is a plot illustrating the proportion of each base position for which each of the four normal DNA bases has been called during sequence analysis. Medium gray: % T; dark gray: % C; light gray: % A and Black: % G.

FIG. 30 is a plot illustrating the per sequence GC content, i.e. the average GC content of reads. Normal random libraries typically have a roughly normal distribution of GC content. The Positive sample is shown in light gray (top peak), the Test sample is shown in dark gray (bottom peak).

FIG. 31 is a plot showing the percentage of base calls at each position for which “N” was called.

FIG. 32 is a plot illustrating the sequence duplication levels. The plot shows the relative level of duplication found for every sequence.

FIG. 33 is a plot illustrating the total amount of over-represented sequences found in each library.

FIG. 34 is a diagram illustrating an exemplary method of the disclosure. Nucleic acids in the sample are adapter ligated, and then cleaved with a nucleic acid-guided nuclease that cleaves the nucleic acids targeted for depletion, resulting in nucleic acids of interest that are adapter ligated on both ends. This method can be used in conjunction with the ligation free library preparation methods of the disclosure.

### DETAILED DESCRIPTION OF THE INVENTION

Capturing information from trace nucleic acid samples, or degraded samples comprising small nucleic acid fragments, remains a significant challenge, particularly for the field of DNA forensics. These samples generally contain nucleic acid fragments that are too small for traditional PCR. Further, the amount of nucleic acids in the sample may be too small for traditional ligation-based based methods library preparation, which are inefficient. However, high-throughput sequencing (HTS) has the potential to recover information from these samples, as even small fragments can contain single nucleotide polymorphisms (SNPs) or other markers useful for identification, predicting visible characteristics such as ancestry and hair/eye color, and generating investigative leads. Disclosed herein are methods of ligation-free library preparation that can be optionally combined with targeted enrichment and/or depletion strategies that, coupled with custom informatics methods, can generate investigative leads from highly-degraded forensic samples.

Guide nucleic acids (gNAs), including guide RNAs (gRNAs) and guide DNAs (gDNAs) for targeting of CRISPR/Cas system proteins to target sites in nucleic acids (e.g., genomic DNA or cDNA) are of tremendous use in a variety of downstream applications, including clinical or diagnostic studies, as well as research. Collections of gNAs can be used with the ligation-free library preparation methods described herein to target sequences in the library for depletion, and thereby enrich for sequences of interest SNPs or other markers.

The disclosure provides methods for the efficient and cost-effective generation of gNAs and libraries of gNAs. Generating libraries of gNAs, e.g. gRNAs, often involves in vitro RNA transcription from a DNA template or library of DNA templates. However, RNA polymerases used to in vitro transcribe gRNAs, such as T7, T3 or SP6 polymerases, frequently fail to precisely terminate transcription and add additional random nucleotides to the 3′ end of transcribed RNAs that do not correspond to the DNA template (referred to herein as untemplated nucleotides). For Cas9 system compatible gRNAs, these additional untemplated 3′ nucleotides in the gRNA are added after the protein binding stem-loop stem sequence. Because of their location in the Cas9 gRNA, these additional nucleotides are unlikely to affect targeting of the Cas9 nucleic acid-guided nuclease-gRNA complex to its target, or cutting of the target sequence. However, for Cpf1 compatible gRNAs, the protein binding stem loop sequence of the gRNA is located 5′ of the target sequence, and so the untemplated 3′ nucleotides added by polymerases such as T7 are added immediately downstream of the target recognition sequence, where these untemplated nucleotides can affect the function of the Cpf1 nucleic acid-guided nuclease-gRNA complex. There thus exists a need in the art for in vitro transcribed RNAs that do not comprise additional 3′ untemplated nucleotides. The invention provides compositions and methods for removing untemplated nucleotides from the 3′ end of in vitro transcribed RNAs.

The “nucleic acid-guided nuclease-gRNA complex” refers to a complex comprising a nucleic acid-guided nuclease protein and a guide RNA. For example, the “Cpf1-gRNA complex” refers to a complex comprising a Cpf1 protein and a gRNA. The nucleic acid-guided nuclease may be any type of nucleic acid-guided nuclease, including but not limited to wild type nucleic acid-guided nuclease, a catalytically dead nucleic acid-guided nuclease, a nucleic acid-guided nuclease-nickase, and nucleases such as Cas9, Cpf1 and variants thereof.

The term “next-generation sequencing” refers to the so-called parallelized sequencing-by-synthesis or sequencing-by-ligation platforms, for example, those currently employed by Illumina, Life Technologies, and Roche, etc. Next-generation sequencing methods may also include nanopore sequencing methods or electronic-detection based methods such as Ion Torrent technology commercialized by Life Technologies.

The term “RNA promoter adapter” is an adapter that contains a promoter for a bacteriophage RNA polymerase, e.g., the RNA polymerase from bacteriophage T3, T7, SP6 or the like.

### Ligation-Free Preparation of Nucleic Acids by Single-Sided PCR

The disclosure provides methods of preparing libraries of nucleic acids, sometimes referred to herein as collections, without ligating adapters to the nucleic acids. The ligation-free methods of the instant disclosure allow for the capture of small fragments (e.g., less than 50 bp) in libraries, e.g. sequencing libraries. Thus, the methods of the instant disclosure are superior in their ability to capture small, trace and/or highly degraded nucleic acid samples in sequencing libraries for analysis when compared to convention methods of library preparation, which rely on adapter ligation. The libraries described herein can be used for sequencing, including high-throughput sequencing.

Capturing information from trace and degraded nucleic acid samples remains a significant challenge, particularly for the field of DNA forensics, but also for other fields such as archaeology and ancient DNA, and cell-free nucleic acids. These samples generally contain nucleic acids in fragments that are too small for traditional PCR and are thus not amenable to Combined DNA Index System (CODIS) profiling. Furthermore, the samples may not even contain complete copies of the donor's genome. High-throughput sequencing has the potential to recover information from these samples, as even small fragments can contain single nucleotide polymorphisms (SNPs) or other markers useful for identification, predicting visible characteristics such as ancestry and hair/eye color, and generating investigative leads.

Disclosed herein are methods of ligation-free library preparation that can be optionally combined with a targeted enrichment strategy that, coupled with custom informatics methods, can generate investigative leads from highly-degraded forensic samples.

In some embodiments, the methods of disclosure comprise (a) extracting nucleic acids using a protocol optimized to retain small fragments; (b) applying one of the ligation-free library preparation methods disclosed herein, wherein the method is targeted to a pre-selected panel of forensically relevant SNPs; (c) sequencing the library with high-throughput sequence methods; and (d) using custom informatics methods to generate a report that includes sex, autosomal ancestry, maternal and paternal lineage, select phenotypic markers, and match probabilities with confidence levels. In some embodiments, the library prepared using the ligation-free methods described herein is subject to depletion of sequences targeted for depletion prior to sequencing, thereby enriching for sequences of interest. For example, a sequencing library from a human forensics sample can be contacted with a plurality of gNAs and CRISPR/Cas system proteins prior to sequencing, wherein the plurality of gNAs target sequences for depletion, for example, human sequences excluding sequences comprising forensically relevant SNPs or other markers.

The targeted primer extension-based sequencing methods of the disclosure involve the use of a single primer binding near a sequence of interest (for example, a SNP or miniSTR). This approach bypasses the need for two primer binding sites in a fragment (e.g., in PCR), enabling the inclusion of very small (<50 base pair) fragments. Furthermore, sequencing adapters are added without the need for ligation, which is known to be highly inefficient and results in sample loss.

Targeted sequencing using the methods described herein can be conducted without ligation of adapters. This can enable sequencing of otherwise difficult to sequence samples, such as highly degraded samples. Highly degraded DNA, in addition to containing primarily short fragments, often has cross-links to other molecules, making the end-to-end amplification required for sequencing libraries inefficient or impossible. Additionally, existing protocols can require conversion of the entire sample to DNA libraries by ligating adapters, followed by a time-consuming enrichment and multiple PCR amplifications.

The pipeline described herein can be applied to extract information from samples for which the Combined DNA Index System (CODIS) genotyping failed, and can also provide investigative leads for cases in which no match is found in the CODIS database.

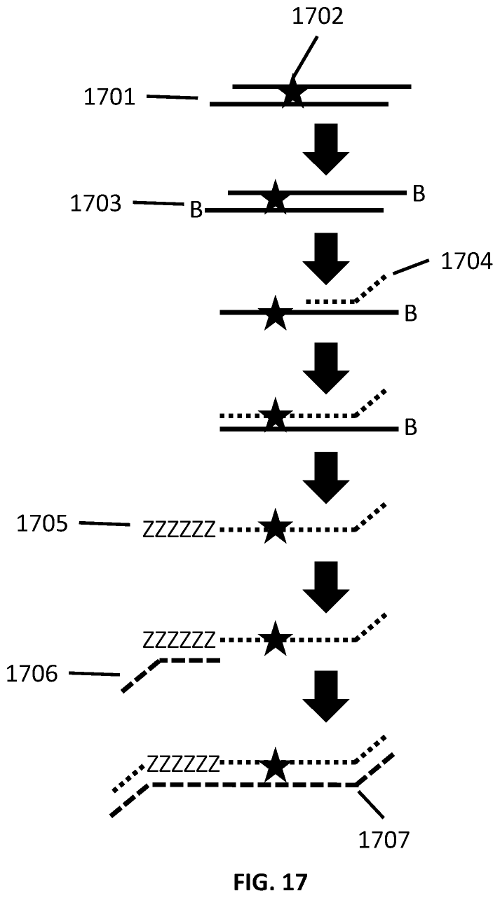

FIG. 17 illustrates a protocol that merges the library generation and enrichment to a single workflow, which can be faster and more efficient at recovering degraded DNA. First, 3′ ends of DNA molecules 1701 in the extract are modified, so they are blocked 1703 and will not be extended by any polymerase. Next, a sequencing adapter-tailed primer 1704 is designed to bind near the site of interest 1702 (most often a SNP, but could be miniSTR or other site), and is extended past the site of interest to the end of the DNA fragment. After removing unused primers, a terminal transferase is added and only the extended primers are given a tail 1705, since other fragments are blocked. Removal of unused primers can be conducted enzymatically (e.g., by digestion with an exonuclease) or by binding of labeled nucleotides (e.g., biotinylated nucleotides) incorporated in the extension. The tail is used to reverse prime with another adapter-containing primer 1706, converting the DNA into a library 1707 ready for amplification and sequencing. For higher sensitivity, a linear amplification step can be added by cycling the first extension step prior to removal of un-extended primer.

Primers can also incorporate barcode or unique molecular identifier (UMI) sequences, enabling tracking of distribution of targeted sites to gain quantitative information, removal of amplification errors, and prevention of cross-contamination from other samples. For example, with two flanking 8-mer UMIs more than 4 billion combinations (416) per primer are possible. As an additional metric, in some applications of the methods, for example those involving restriction digest prior to library preparation, the 3′ breakpoint for the original molecule is known, making it virtually impossible to encounter the same combination multiple times. With a database of previously used UMIs for each primer, contamination from previously handled samples can be monitored. Importantly, these data can be stored without keeping identifiable information to protect privacy.

Such ligation-free library preparation protocols can be used for forensics or other identification of individuals. For example, sequences of interest can include SNPs and other markers in mitochondrial DNA (mtDNA) and Y chromosome sites for assignment of maternal and paternal haplogroups. MiniSTRs or other identifying regions can be employed. For degraded samples, it is often favorable to look at the mitochondrial DNA due to its high copy number and well-characterized haplogroup tree.

Such ligation-free library preparation protocols can be used for disease diagnostics. For example, sequences of interest can include taxonomic markers including clade markers. Sequences of interest can include disease trait markers such as pathogenicity, virulence, resistance, strain identification, and other markers.

The disclosure provides methods of preparing a library of nucleic acids, comprising: (a) providing a sample of nucleic acids comprising at least one target sequence; (b) contacting the sample of nucleic acids, with a plurality of first polymerase chain reaction (PCR) primers and a polymerase under conditions that allow PCR to occur, thereby generating a plurality of first single-sided PCR products; (c) contacting the plurality of first single-sided PCR products with a terminal transferase under conditions sufficient to transfer dNTPs to the 3′ ends of the plurality of first single-sided PCR products, thereby generating a plurality of PCR products comprising 3′ tails; and (d) contacting the plurality of PCR products comprising 3′ tails, a plurality of second PCR primers and a polymerase under conditions that allow PCR to occur; thereby generating a library of nucleic acids with adapters at the 5′ and 3′ ends.

In some embodiments, the methods comprise blunting overhangs of the nucleic acids in the sample prior to the first single-sided PCR reaction. The overhangs can be 5′ or 3′ overhangs, and the nucleic acids comprise double stranded DNA. Blunting is a process in which single-stranded overhangs created by restriction digest or shearing are filled in by addition of nucleotides to the complementary strand, or by removing the overhang with an exonuclease. Exemplary blunting enzymes include T4 polymerase, Klenow fragment or Mung Bean Nuclease. For example, 1 Unit (U) T4 DNA polymerase per μg of sample DNA can be used. Blunting allows for the efficient incorporation of dNTPs or ddNTPs at the ends of DNAs by enzymes such as the Klenow fragment.

In some embodiments, the blunted sample of nucleic acids is purified following blunting.

In an exemplary embodiment, 1 Unit (U) T4 DNA polymerase per μg DNA is used to blunt the sample of nucleic acids. In an exemplary embodiment, the reaction is incubated at 12° C. for 15 minutes, and then at 75° C. for 20 minutes.

Purification can include removal of unincorporated nucleotides (e.g. dNTPs) introduced in the blunting reaction. The blunted sample of nucleic acids can be purified enzymatically, for example by using recombinant shrimp alkaline phosphatase, or using a bead or column-based purification strategy. An exemplary column purification strategy comprises the Qiaquick PCR purification kit, although alternative purification strategies will be known to the person of ordinary skill in the art.

In some embodiments, the methods comprising blocking the 3′ ends blunted sample of nucleic acids. Blocking can be accomplished by using an enzyme to incorporate dideoxynucleotides (ddNTPs) at the 3′ ends of blunted DNAs. In some embodiments, the enzyme is the Klenow fragment. The Klenow fragment is a fragment of DNA polymerase I that retains 5′ to 3′ polymerase activity and 3′ to 5′ exonuclease activity, but does not have 5′ to 3′ exonuclease activity.

In an exemplary embodiment, the sample of nucleic acids is incubated with Klenow, ddNTPs and a suitable buffer for 40 minutes at 37° C., and then for 75° C. for 20 minutes.

In some embodiments, the blocked sample of nucleic acids is purified following blocking. Purification can include removal of unincorporated nucleotides (e.g. ddNTPs) introduced in the blocking reaction. The blocked sample of nucleic acids can be purified enzymatically, for example by using alkaline phosphatase, or using a bead or column-based purification strategy. In some embodiments, the alkaline phosphatase is recombinant shrimp alkaline phosphatase. An exemplary column purification strategy comprises the Qiaquick Nucleotide removal kit, although alternative purification strategies will be known to persons of ordinary skill in the art.

In some embodiments, a first adapter is added to the sample of nucleic acids in a first single-sided PCR reaction using a first PCR primer. Single sided PCR, sometimes referred to as single-sided PCR, uses a single primer that base pairs with and binds to a sequence in a nucleic acid, and is then extended in a templated fashion by a polymerase. In some embodiments, the polymerase is a Klenow Fragment. In some embodiments, the polymerase is a Taq polymerase. In some embodiments, the polymerase is a high-fidelity polymerase, for example a Qiagen high fidelity polymerase. Suitable polymerases will be known to persons of ordinary skill in the art.

In some embodiments, the first PCR primer comprises (i) a sequence complementary to a sequence adjacent to or overlapping the at least one target sequence, and (ii) a first adapter sequence. In some embodiments, the first adapter sequence is 5′ of the sequence complementary to the sequence adjacent to or overlapping the at least one target sequence.

As used herein, “adjacent” refers to a sequence within 1-500, 1-300, 1-100, 1-75, 1-50 or 1-25 nucleotides of another sequence, for example a sequence of interest. Sequences that are “overlapping” can be wholly, or partly overlapping. For example, sequences that overlap by 1, 2, 3, 4, 5, 6, 7, 8, 9, 10, 11, 12, 13, 14, 15, 16, 17, 18, 19, 20, 21, 22, 23 24, 25 or more nucleotides are considered to be overlapping. In an exemplary embodiment, the sequence of interest comprises a forensically interesting SNP, and the first PCR primer binds within 1-20 nucleotides of the SNP.

In some embodiments, the first adapter comprises a first unique molecular identifier (UMI). In some embodiments, the first UMI comprises 2, 3, 4, 5, 6, 7, 8, 9, 10, 11, or 12 nucleotides. In some embodiments, the first UMI is more than 12 nucleotides. In some embodiments, the first UMI comprises or consists essentially of a random sequence.

In some embodiments, the first adapter comprises a sequencing adapter, for example for Illumina sequencing.

In some embodiments, the first adapter comprises a sequence of a NEBNext Adapter. The ordinarily skilled artisan will be able to design adapters suited to particular high-throughput sequencing platforms and applications.

In some embodiments, the first sing-sided PCR product is purified following the first single-sided PCR reaction. Purification can include removal of unincorporated nucleotides (e.g. ddNTPs) introduced in the blocking reaction. The first single-sided PCR product can be purified enzymatically, for example by using alkaline phosphatase, or using a bead or column-based purification strategy. In some embodiments, the alkaline phosphatase is recombinant shrimp alkaline phosphatase. An exemplary column purification strategy comprises the MinElute PCR purification kit, although alternative purification strategies will be known to persons of ordinary skill in the art.

In some embodiments, untemplated dNTPs are added to the 3′ end of the first single-sided PCR product. The untemplated dNTPs can be dATPs (a polyA tail), dCTPs (a polyC tail), dGTPs (a polyG tail) or dTTPs (a polyT tail). In some embodiments, the untemplated 3′ nucleotides are polyGs (G-tailing). G-tailing can provide superior consistency to A-tailing across a variety of sample DNA input concentrations.

Untemplated nucleotides can be added to nucleic acid samples using a terminal transferase. Exemplary terminal transferases include Terminal Transferase (TdT) from NEB.

In an exemplary embodiment, 1:1000 pmol ends to pmol dNTPs are used for the tailing reaction. 0.2 U/μL Terminal transferase up to 5 pmol are used. In an exemplary embodiment, the terminal transferase reactions are incubated at 37° C. for 30 minutes, and then at 70° C. for 10 minutes.

In some embodiments, the tailed single-sided PCR product is purified following tailing. Purification can include removal of unincorporated nucleotides (e.g. dNTPs) introduced in the terminal transferase reaction. The tailed first single-sided PCR product can be purified enzymatically, for example by using alkaline phosphatase, or using a bead or column-based purification strategy. In some embodiments, the alkaline phosphatase is recombinant shrimp alkaline phosphatase. An exemplary column purification strategy comprises the MinElute Reaction cleanup kit, although alternative purification strategies will be known to persons of ordinary skill in the art.

In some embodiments, a second adapter is added to the sample of nucleic acids in a second single-sided PCR reaction following 3′ tailing. In some embodiments, the polymerase is a Taq polymerase. In some embodiments, the polymerase is a high-fidelity polymerase, for example a Qiagen high fidelity polymerase. Suitable polymerases will be known to persons of ordinary skill in the art.

In some embodiments, the second PCR primer for the second PCR reaction comprises (i) a sequence complementary to the 3′ tails added to first PCR products at the tailing step, and (ii) a second adapter sequence. For example, if the tailing step added polyG tails to the nucleic acids in the sample, the second PCR primer comprises a polyC sequence to facilitate base-pairing with the polyG tails. In some embodiments, the second adapter sequence is 5′ of the sequence complementary to the 3′ tail.

In some embodiments, the second adapter comprises a second unique molecular identifier (UMI). In some embodiments, the second UMI comprises 2, 3, 4, 5, 6, 7, 8, 9, 10, 11, or 12 nucleotides. In some embodiments, the second UMI is more than 12 nucleotides. In some embodiments, the second UMI comprises or consists essentially of a random sequence. In some embodiments, the first and second UMI sequences are the same sequence. In some embodiments, the first and second UMI sequences are not the same sequence.

In some embodiments, the second adapter comprises a sequencing adapter, for example for Illumina sequencing.

In some embodiments, the second adapter comprises a sequence of a NEBNext Adapter. The ordinarily skilled artisan will be able to design adapters suited to particular high-throughput sequencing platforms and applications.

In some embodiments, the second single-sided PCR product is purified following the second single-sided PCR reaction.

In some embodiments, the second single-sided PCR product can be purified using a bead or column-based purification strategy. Purification can include removal of unincorporated nucleotides (e.g. ddNTPs) introduced in the second single-sided PCR reaction. An exemplary column purification strategy comprises the MinElute PCR purification kit, although alternative purification strategies will be known to persons of ordinary skill in the art.

In some embodiments, indexing sequences are added to the second single-sided PCR product in an indexing PCR reaction. For example, in those embodiments where the first and second adapters do not comprise UMI sequences, indexing sequences comprising UMI sequences, and optionally, additional adapter sequences tailored to particular high-throughput sequencing platforms can be added in an indexing PCR reaction.

In some embodiments, the methods comprise contacting the plurality of PCR products from the second single-sided PCR reaction with a plurality of first indexing primers, a plurality of second indexing primers, and a polymerase under conditions that allow PCR to occur.

In some embodiments, first indexing primer comprises a sequence complementary to the first adapter and a first unique molecular identifier sequence (UMI). For example, if the first adapter comprises a sequence of a NEBNext adapter, the indexing primer comprises a sequence complementary to the NEBNext adapter sequence of the first adapter. In some embodiments, the first UMI sequence is 5′ of the sequence complementary to the first adapter. In some embodiments, the first UMI comprises 2, 3, 4, 5, 6, 7, 8, 9, 10, 11, or 12 nucleotides. In some embodiments, the first UMI is more than 12 nucleotides. In some embodiments, the first UMI comprises or consists essentially of a random sequence. In some embodiments, the first indexing primer comprises a sequencing adapter, for example for Illumina sequencing.

In some embodiments, the second indexing primer comprises a sequence complementary to the second adapter and a second UMI sequence. For example, if the second adapter comprises a sequence of a second NEBNext adapter, the second indexing primer comprises a sequence complementary to the second NEBNext adapter sequence of the second adapter. In some embodiments, the second UMI sequence is 5′ of the sequence complementary to the second adapter. In some embodiments, the second UMI comprises 2, 3, 4, 5, 6, 7, 8, 9, 10, 11, or 12 nucleotides. In some embodiments, the second UMI is more than 12 nucleotides. In some embodiments, the second UMI comprises or consists essentially of a random sequence. In some embodiments, the first and second UMI sequences are the same sequence. In some embodiments, the first and second UMI sequences are not the same sequence.

In some embodiments, the second indexing primer comprises a sequencing adapter, for example for Illumina sequencing. The ordinarily skilled artisan will be able to design indexing primers suited to particular high-throughput sequencing applications.

In an embodiment, the indexing PCR reaction comprises 6 polymerase extension cycles. The number of polymerase extension cycles can be calculated based off of qPCR plateau values quantifying the amount of PCR product from the second single-sided PCR reaction.

In some embodiments, the indexing PCR product is purified following indexing PCR. In some embodiments, the purification comprises Kapa Pure beads (Roche).

In some embodiments, libraries generated using the methods disclosed herein can be further processed according to the methods of depletion/enriched of the instant disclosure. For example, sequences for depletion in the library can be targeted using collections of gNAs, which direct a nucleic-acid guided nuclease to sequences targeted for depletion in the library.

High-throughput sequencing data generated using the methods described herein can be analyzed using any methods known in the art. Software tools for analyzing high-throughput sequencing data include, but are not limited to, Samtools, FastQC, BWA, GenomeMapper, Novoalign, mrsFAST, Bowtie, GEM mapper, MoDIL, BreakDancer, Splitread, DeNovoGear and Scalpel.

Sites of interest can be used to determine identity of a subject. In some cases, identity can be determined using identity by state (IBS) or identity-by-decent (IBD). In identifying different genealogical relationships, relationship can be defined as R=(k₀, k₁, k₂), where km matches the fraction of the genome where the two individuals share m alleles. Table 1 has expected values for relationships typically relevant in forensics. This can be formulated in Bayesian terms as:

R=((IBD=k₀|Data),(IBD=k₁|Data,P(IBD=k₂|Data).

Combining this with the expected values from table 1, we can setup a likelihood ratio test as:

$$LR = \frac{L ( H ( Data ) )}{L ( H ( Expected ) )} = {\prod }_{i = 0}^{2} \frac{P ( IBD = {k}_{i} \vert Data )}{P ( IBD = {k}_{i} \vert Expected )}$$

A measure of significance is the obtained by making use of the following asymptotic property:

$$- 2 log ( LR ) ∼ {χ}_{d}^{2}$$

where d is degrees of freedom.

TABLE 1
Expected allele sharing among related individuals.

| Relationship | k0 | k1 | k2 |
|---|---|---|---|
| Self/mono-zygotic | 0 | 0 | 1 |
| twin | | | |
| Parent-Offspring | 0 | 1 | 0 |
| Full Siblings | 0.25 | 0.5 | 0.25 |
| Niece, nephew, uncle, | 0.5 | 0.5 | 0 |
| aunt, grandparent, | | | |
| grandchild, | | | |
| half-sibling | | | |
| First cousins | 0.75 | 0.25 | 0 |
| Unrelated | 1 | 0 | 0 |

High-throughput sequencing can enable analysis of a huge pool of degraded/trace forensics samples that are refractory to current STR-based genotyping methods. The SNP data generated by HTS also contains information that STR profiles do not, including ancestry and phenotype predictions that can be used to generate investigative leads. As such, the methods disclosed herein can serve as a supplement for samples where partial or no CODIS profile can be generated, and can add additional data for investigative leads in cases where no match is found in the CODIS database. However, for the forensics community to transition to HTS, it needs the tools to collect and analyze SNP data in the most efficient, inexpensive, and targeted way possible. The methods disclosed herein can give a reliable way of testing highly degraded samples, by focusing extraction methods on shorter DNA fragments and targeting sequencing to sites of interest, followed by analysis with a streamlined informatics pipeline backed by strong statistical analyses.

### Ligation-Free Preparation of Nucleic Acids by Strand Switching

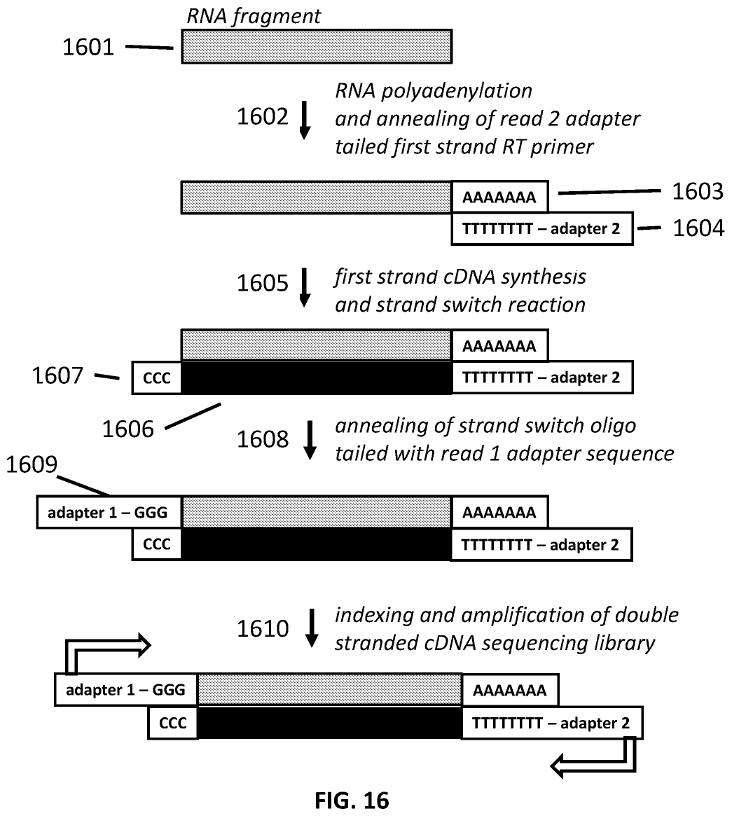

RNA can be prepared for sequencing (e.g., as cDNA) using a strand-switching method. FIG. 16 shows an exemplary schematic of such a strand-switching method. RNA molecules 1601 can be polyadenylated 1602 or otherwise given a tail (e.g., a poly-A tail) 1603. An oligonucleotide comprising an adapter (here, “Adapter 2”) 1604 can be hybridized to the RNA tail, for example via a poly-T region of the oligonucleotide. Reverse transcription 1605 can then be used to synthesize cDNA 1606. A region such as a poly-C region 1607 can be added to the cDNA for example by using MMLV as the reverse transcriptase, which can enable strand-switching. A strand-switching oligonucleotide 1609 can then be hybridized to the cDNA tail (e.g., the poly-C tail), for example via a poly-G region of the oligonucleotide. The strand-switching oligonucleotide can comprise an adapter (here, “Adapter 1”). The adapters can then be used for amplification and/or indexing 1610 of a double stranded cDNA sequencing library.

The adapters can comprise sequencing adapters (e.g., Illumina sequencing adapters). The adapters can comprise unique molecular identifier (UMI) sequences. The UMI sequences can comprise a sequence that is unique to each original RNA molecule (e.g., a random sequence). In some embodiments, the UMI comprises 2, 3, 4, 5, 6, 7, 8, 9, 10, 11, or 12 nucleotides. In some embodiments, the UMI is more than 12 nucleotides. In some embodiments, the UMI comprises or consists essentially of a random sequence. This can allow quantification of RNA amounts, free from sequencing bias. The adapters can comprise “barcode” sequences. The barcode sequences can comprise a barcode sequence that is shared among RNA molecules from a particular source (such as a subject, patient, environmental sample, partition (e.g., droplet, well, bead)). This can allow pooling of sequencing information for subsequent analysis, and can allow detection and elimination of cross-contamination. The adapters can comprise multiple distinct sequences, such as a UMI unique to each RNA molecule, a barcode shared among RNA molecules from a particular source, and a sequencing adapter.

The cDNA library can be further processed according to methods of the present disclosure, such as by targeted digestion or other depletion. For example, cDNA from a host (e.g., a human) can be digested or otherwise depleted, while cDNA from a non-host (e.g., an infectious agent) can remain. The cDNA can be sequenced or otherwise analyzed (e.g., hybridization assay, amplification assay).

Collections of gRNAs, nucleic acid-guided nucleases, or complexes thereof can be arranged on one or more surfaces. Arrangement on surfaces can be used to control the amount, timing, and/or order with which a sample encounters the gRNAs, nucleic acid-guided nucleases, or complexes thereof. For example, gRNAs, nucleic acid-guided nucleases, or complexes thereof can be bound to the surface of a channel into which a sample is flowed; gRNAs, nucleic acid-guided nucleases, or complexes thereof bound to the surface closer to the beginning of the channel will be encountered before those bound toward the end of the channel. In some cases, this approach can be used to cause a sample to encounter gRNAs, nucleic acid-guided nucleases, or complexes thereof targeted to the most frequent recognition sequences, which can be designed and produced as discussed herein. In some cases, this approach can be used to cause a sample to encounter gRNAs, nucleic acid-guided nucleases, or complexes thereof in different amounts or relative amounts, such as in proportion to the frequency of the gRNA in the target nucleic acid. In an example, a first gRNA-nucleic acid-guided nuclease complex is targeted to a sequence that appears twice as frequently in a target genome compared to a second gRNA-nucleic acid-guided nuclease complex, and twice the number of the first complex is bound to a surface compared to the number of the second complex bound to the surface.

Collections of gRNAs, nucleic acid-guided nucleases, or complexes thereof can be bound to a variety of surfaces, including but not limited to arrays, flow cells, channels, microfluidic channels, beads, and other substrates.

### Methods of Library Depletion Enrichment

In some embodiments, libraries of nucleic acids are depleted of nucleic acids targeted for depletion, and thereby enriched for nucleic acids comprising sequences of interest prior to high throughput sequencing.

In some embodiments, the collections of gNAs provided herein, and the methods of depleting sequences targeted for depletion, partitioning, capturing or enriching sequences of interest can be combined the methods of ligation-free preparation of nucleic acid libraries described herein. In some embodiments, the sample of nucleic acids comprises RNA, and the ligation-free preparation comprises reverse transcription with template switching. In some embodiments, the sample of nucleic acids comprises DNA, and the ligation-free preparation comprises two single-sided PCR reactions. In some embodiments, the samples of nucleic acids are prepared for downstream applications such as sequencing, high-throughput sequencing, amplification and cloning.

Applications of gNAs including depletion and capture are described in PCT publications WO/2016/100955 and WO/2017/031360, the contents of each of which are hereby incorporated by reference in their entirety.

In one embodiment, the gNAs are selective for host nucleic acids in a biological sample from a host, but are not selective for non-host nucleic acids in the sample from a host. In one embodiment, the gNAs are selective for non-host nucleic acids from a biological sample from a host but are not selective for the host nucleic acids in the sample. In one embodiment, the gNAs are selective for both host nucleic acids and a subset of the non-host nucleic acids in a biological sample from a host. For example, where a complex biological sample comprises host nucleic acids and nucleic acids from more than one non-host organisms, the gNAs may be selective for more than one of the non-host species. In such embodiments, the gNAs are used to serially deplete or partition the sequences that are not of interest. For example, saliva from a human contains human DNA, as well as the DNA of more than one bacterial species, but may also contain the genomic material of an unknown pathogenic organism. In such an embodiment, gNAs directed at the human DNA and the known bacteria can be used to serially deplete the human DNA, and the DNA of the known bacterial, thus resulting in a sample comprising the genomic material of the unknown pathogenic organism.

In an exemplary embodiment, the gNAs are selective for human host DNA obtained from a biological sample from the host, but do not hybridize with DNA from an unknown pathogen(s) also obtained from the sample.

In some embodiments, the sample is a forensic sample, and the gNAs are selective for human sequences that are not of interest in forensic analysis. For example, the gNAs are selective for human sequences that cannot be used to identify individual subjects, i.e. sequences that are highly similar or identical across human populations. This includes, sequences other than SNPs, mini short tandem repeats, Y chromosome markers and X chromosome markers that vary between individual subjects in a population.

In some embodiments, the gNAs are useful for depleting and partitioning of targeted sequences in a sample, enriching a sample for non-host nucleic acids, or serially depleting targeted nucleic acids in a sample comprising: providing nucleic acids extracted from a sample; and contacting the sample with a plurality of complexes comprising (i) any one of the collection of gNAs described herein and (ii) nucleic acid-guided nuclease (e.g., CRISPR/Cas) system proteins.

In some embodiments, the gNAs are useful for methods of depletion and partitioning of targeted sequences in a sample comprising: providing nucleic acids extracted from a sample, wherein the extracted nucleic acids comprise sequences of interest and targeted sequences for one of depletion and partitioning; contacting the sample with a plurality of complexes comprising (i) a collection of gNAs provided herein; and (ii) nucleic acid-guided nuclease (e.g., CRISPR/Cas) system proteins, under conditions in which the nucleic acid-guided nuclease system proteins cleave the nucleic acids in the sample.

In some cases, fusion proteins comprising domains from a nucleic acid-guided nuclease system protein (e.g., a CRISPR/Cas system protein) can be used with gNAs. Domains from nucleic acid-guided nuclease system proteins can include guide nucleic acid complexing domains, target nucleic acid recognition and binding domains, nuclease domains, and other domains. Domains can be from different variants of nucleic acid-guided nuclease system proteins, including but not limited to catalytically active variants, nickase variants, catalytically dead variants, and combinations thereof. Other domains in fusion proteins can come from proteins including restriction enzymes, other endonucleases (e.g., FokI), enzymes that modify DNA (e.g., methyltransferases), or tags (e.g., avidin, or fluorescent proteins such as GFP). As an example, nucleic acid-guided nuclease system protein domains for complexing with guide nucleic acids and binding to target nucleic acids can be combined in a fusion protein with nucleic acid cleaving or nicking domains from restriction enzymes. In some cases, the fusion protein comprises a catalytic domain of a restriction enzyme plus a nucleic acid guided nuclease domain. In some cases, the fusion protein comprises a catalytic domain of a restriction enzyme plus a catalytically-dead nucleic acid guided nuclease domain. For example, the catalytic domain of a restriction enzyme can be a catalytic domain of FokI. The nucleic acid guided nuclease domain can be a Cpf1 or Cas9 domain, including a catalytically dead Cpf1 or Cas9 domain. In some cases, the fusion protein comprises a catalytic domain of a restriction enzyme plus a nucleotide sequence recognition domain. In some cases, the fusion protein comprises a restriction enzyme domain plus a nucleic acid guided nuclease domain. The restriction enzyme domain can be a mutant that lacks a functioning nucleotide sequence recognition domain. For example, the restriction enzyme domain can be FokI, in some cases with a N13Y mutation to inactivate the nucleotide sequence recognition domain. In some cases, the fusion protein comprises a restriction enzyme domain plus a catalytically-dead nucleic acid guided nuclease domain. In some cases, the fusion protein comprises a restriction enzyme domain plus a nucleotide sequence recognition domain. The nucleotide sequence recognition domain can be from a restriction enzyme or a nucleic acid guided nuclease, for example.

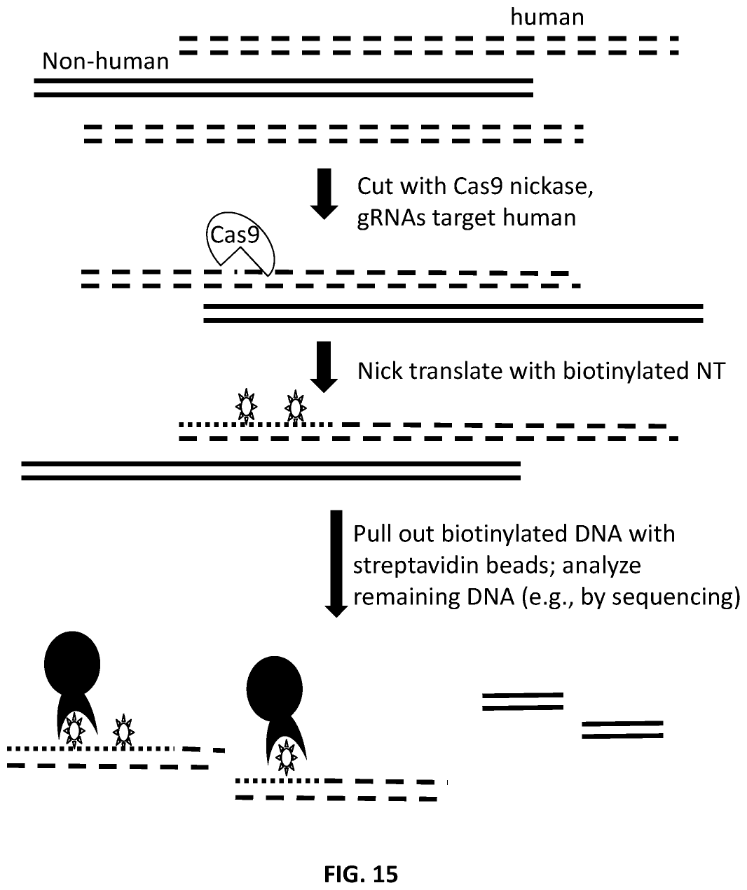

In some embodiments, the gNAs are useful for depleting, partitioning, or capturing targeted nucleic acids (e.g., host nucleic acids) in a sample. For example, gNAs, comprising targeting sequences directed at the target (e.g., host) nucleic acids, are complexed with nucleic acid guided nickase system proteins and used to nick the target nucleic acids. Nick translation can then be conducted with labeled nucleotides, such as biotinylated nucleotides. The labeled nucleic acid sequences generated by nick translation can be used to bind the targeted sequences, such as with streptavidin. This binding can be used to capture the target nucleic acids. The captured target nucleic acids can then be separated from the non-captured nucleic acids. The non-captured nucleic acids (e.g., non-host nucleic acids) can be further analyzed, such as by sequencing. Alternatively or additionally, the captured target nucleic acids can also be further analyzed. FIG. 15 shows an exemplary schematic of such a method. In FIG. 15, a sample comprising human and non-human nucleic acids is contacted with a nucleic acid guided nuclease nickase (e.g., Cas9 nickase) guided by human-targeted guide nucleic acids (e.g., gRNAs). At the nicked sites, nick translation is performed with labeled nucleotides (e.g., biotinylated nucleotides), and the labeled (e.g., biotinylated) nucleic acids can be captured using the labels (e.g., on a streptavidin substrate). The remaining non-human nucleic acids can then be further analyzed, for example by sequencing or other assay (e.g., hybridization, PCR).

Nucleic acids with hairpin loops (e.g., nanopore sequencing adapters) can also be targeted for depletion. A collection of nucleic acids (e.g., a sequencing library) with loops on one side of the nucleic acids (e.g., sequencing adapters) can be obtained. Then, second loops can be added to the other side of the nucleic acids, making the nucleic acids circular. The second loops can comprise a known restriction site or a particular nucleic acid-guided nuclease site. The collection of circular nucleic acids can then be contacted with target-specific (e.g., host-specific, human-specific) nucleic acid-guided nucleases or nickases. These nucleic acid-guided nucleases or nickases can cut or nick the targeted constituents of the nucleic acid collection while leaving the other nucleic acids in the collection intact. The cut or nicked nucleic acids can then be digested with exonucleases, while the intact nucleic acids remain undigested, thereby depleting the targeted nucleic acids from the collection. Then, the second loops can be removed by digestion at the restriction site or particular nucleic acid-guided nuclease site. The non-depleted nucleic acids (e.g., non-host nucleic acids) can then be further analyzed, such as by sequencing (e.g., sequencing on a nanopore sequencing platform). The adapters, such as the second loops, can also be designed such that any adapter dimers formed would result in a known site (e.g., a restriction enzyme site or a specific nucleic acid-guided nuclease site) in the adapter dimers, which can be digested by the appropriate restriction enzyme or nucleic acid-guided nuclease. Such an approach can also be employed for sequencing libraries for sequencing platforms that do not employ hairpin adapters, such as Illumina libraries, for example by amplifying the library after digesting the second loops.

In some embodiments, nucleic acids targeted for depletion can comprise human ribonucleic acids. In some cases, all human ribonucleic acids can be targeted for depletion. In some embodiments, only human ribonucleic acids that are not of forensic or diagnostic interest are targeted for depletion.

In some embodiments, nucleic acids targeted for depletion comprise nucleic acids that are common or prevalent in a subject. For example, the depleted nucleic acids can comprise nucleic acids common to all cell types, or more abundant in typical or healthy cells, including but not limited to those associated with immune system factors (e.g., mRNA). Following depletion, the remaining nucleic acids to be analyzed can then comprise less common or less prevalent nucleic acids, such as cell type-specific nucleic acids. These less common nucleic acids can be signals of cell death, including cell death of one or more particular cell types. Such signals can be indicative of infections, cancers, and other diseases. In some cases, the signals are signals of cancer-related apoptosis in a particular tissue or tissues.

In some embodiments, the gNAs are useful for enriching a sample for non-host nucleic acids comprising: providing a sample comprising host nucleic acids and non-host nucleic acids; contacting the sample with a plurality of complexes comprising (i) a collection of gNAs provided herein comprising targeting sequences directed at the host nucleic acids; and (ii) nucleic acid-guided nuclease (e.g., CRISPR/Cas) system proteins, under conditions in which the nucleic acid-guided nuclease system proteins cleave the host nucleic acids in the sample, thereby depleting the sample of host nucleic acids, and allowing for the enrichment of non-host nucleic acids.

In some embodiments, the gNAs are useful for one method for serially depleting targeted nucleic acids in a sample comprising: providing a biological sample from a host comprising host nucleic acids and non-host nucleic acids, wherein the non-host nucleic acids comprise nucleic acids from at least one known non-host organism and nucleic acids from an unknown non-host organism; providing a plurality of complexes comprising (i) a collection of gNAs provided herein, directed at the host nucleic acids; and (ii) nucleic acid-guided nuclease (e.g., CRISPR/Cas) system proteins; mixing the nucleic acids from the biological sample with the gRNA-nucleic acid-guided nuclease system protein complexes (e.g., gNA-CRISPR/Cas system protein complexes) configured to hybridize to targeted sequences in the host nucleic acids, wherein at least a portion of the complexes hybridizes to the targeted sequences in the host nucleic acids, and wherein at least a portion of the host nucleic acids are cleaved; mixing the remaining nucleic acids from the biological sample with the gNA-nucleic acid-guided nuclease system protein complexes configured to hybridize to targeted sequences in the at least one known non-host nucleic acids, wherein at least a portion of the complexes hybridizes to the targeted sequences in the at least one non-host nucleic acids, and wherein at least a portion of the non-host nucleic acids are cleaved; and isolating the remaining nucleic acids from the unknown non-host organism and preparing for further analysis.

In some embodiments, the gNAs generated herein are used to perform genome-wide or targeted functional screens in a population of cells. In such an embodiment, libraries of in vitro-transcribed gRNAs or vectors encoding the gRNAs can be introduced into a population of cells via transfection or other laboratory techniques known in the art, along with a nucleic acid-guided nuclease (e.g., CRISPR/Cas) system protein, in a way that gNA-directed nucleic acid-guided nuclease system protein editing can be achieved to sequences across the entire genome or to a specific region of the genome. In one embodiment, the nucleic acid-guided nuclease system protein can be introduced as a DNA. In one embodiment, the nucleic acid-guided nuclease system protein can be introduced as mRNA. In one embodiment, the nucleic acid-guided nuclease system protein can be introduced as protein. In one exemplary embodiment, the nucleic acid-guided nuclease system protein is Cpf1. In one exemplary embodiment, the nucleic acid-guided nuclease system protein is Cas9.

In some embodiments, the gNAs generated herein are used for the selective capture and/or enrichment of nucleic acid sequences of interest. For example, in some embodiments, the gNAs generated herein are used for capturing target nucleic acid sequences comprising: providing a sample comprising a plurality of nucleic acids; and contacting the sample with a plurality of complexes comprising (i) a collection of gNAs provided herein; and (ii) nucleic acid-guided nuclease (e.g., CRISPR/Cas) system proteins. Once the sequences of interest are captured, they can be further ligated to create, for example, a sequencing library.

In some embodiments, the gNAs generated herein are used for introducing labeled nucleotides at targeted sites of interest comprising: (a) providing a sample comprising a plurality of nucleic acid fragments; (b) contacting the sample with a plurality of complexes comprising (i) a collection of gNAs provided herein; and (ii) nucleic acid-guided nuclease (e.g., CRISPR/Cas) system protein-nickases (e.g. Cas9-nickases or Cpf1-nickases), wherein the gNAs are complementary to targeted sites of interest in the nucleic acid fragments, thereby generating a plurality of nicked nucleic acid fragments at the targeted sites of interest; and (c) contacting the plurality of nicked nucleic acid fragments with an enzyme capable of initiating nucleic acid synthesis at a nicked site, and labeled nucleotides, thereby generating a plurality of nucleic acid fragments comprising labeled nucleotides in the targeted sites of interest.

In some embodiments, the gNAs generated herein are used for capturing target nucleic acid sequences of interest comprising: (a) providing a sample comprising a plurality of adapter-ligated nucleic acids, wherein the nucleic acids are ligated to a first adapter at one end and are ligated to a second adapter at the other end; and (b) contacting the sample with a collection of gNAs which comprise a plurality of dead nucleic acid-guided nuclease-gNA complexes (e.g., dCpf1-gRNA complexes), wherein the dead nucleic acid-guided nuclease (e.g., dCpf1) is fused to a transposase, wherein the gNAs are complementary to targeted sites of interest contained in a subset of the nucleic acids, and wherein the dead nucleic acid-guided nuclease-gNA transposase complexes (e.g., dCpf1-gRNA transposase complexes) are loaded with a plurality of third adapters, to generate a plurality of nucleic acids fragments comprising either a first or second adapter at one end and a third adapter at the other end. In one embodiment the method further comprises amplifying the product of step (b) using first or second adapter and third adapter-specific PCR.

In some embodiments, the gNAs generated herein are used to perform genome-wide or targeted activation or repression in a population of cells. In such an embodiment, libraries of in vitro-transcribed gNAs or vectors encoding the gNAs can be introduced into a population of cells via transfection or other laboratory techniques known in the art, along with a catalytically dead nucleic acid-guided nuclease (e.g., CRISPR/Cas) system protein fused to an activator or repressor domain (catalytically dead nucleic acid-guided nuclease system protein-fusion protein), in a way that gRNA-directed catalytically dead nucleic acid-guided nuclease system protein-mediated activation or repression can be achieved at sequences across the entire genome or to a specific region of the genome. In one embodiment, the catalytically dead nucleic acid-guided nuclease system protein-fusion protein can be introduced as DNA. In one embodiment, the catalytically dead nucleic acid-guided nuclease system protein-fusion protein can be introduced as mRNA. In one embodiment, the catalytically dead nucleic acid-guided nuclease system protein-fusion protein can be introduced as protein. In some embodiments, the collection of gNAs or nucleic acids encoding for gNAs exhibit specificity for more than one nucleic acid-guided nuclease system protein. In one exemplary embodiment, the catalytically dead nucleic acid-guided nuclease system protein is dCpf1.

In some embodiments, the collection comprises gNAs or nucleic acids encoding for gNAs with specificity for Cpf1 and one or more CRISPR/Cas system proteins selected from the group consisting of Cas9, Cas3, Cas8a-c, Cas10, Cse1, Csy1, Csn2, Cas4, Csm2, CasX, CasY, Cas13, Cas14 and Cm5. In some embodiments, the collection comprises gNAs or nucleic acids encoding for gNAs with specificity for various catalytically dead CRISPR/Cas system proteins fused to different fluorophores, for example for use in the labeling and/or visualization of different genomes or portions of genomes, for use in the labeling and/or visualization of different chromosomal regions, or for use in the labeling and/or visualization of the integration of viral genes/genomes into a genome.

In some embodiments, the collection of gNAs (or nucleic acids encoding for gNAs) have specificity for different nucleic acid-guided nuclease (e.g., CRISPR/Cas) system proteins, and target different sequences of interest, for example from different species. For example, a first subset of gNAs from a collection of gNAs (or transcribed from a population of nucleic acids encoding such gRNAs) targeting a genome from a first species can be first mixed with a first nucleic acid-guided nuclease system protein member (or an engineered version); and a second subset of gNAs from a collection of gNAs (or transcribed from a population of nucleic acids encoding such gNAs) targeting a genome from a second species can be mixed with a second different nucleic acid-guided nuclease system protein member (or an engineered version). In one embodiment, the nucleic acid-guided nuclease system proteins can be a catalytically dead version (for example dCpf1) fused with different fluorophores, so that different targeted sequence of interest, e.g. different species genome, or different chromosomes of one species, can be labeled by different fluorescent labels. For example, different chromosomal regions can be labeled by different gNA-targeted dCpf1-fluorophores, for visualization of genetic translocations. For example, different viral genomes can be labeled by different gNA-targeted dCpf1-fluorophores, for visualization of integration of different viral genomes into the host genome. In another embodiment, the nucleic acid-guided nuclease system protein can be dCpf1 fused with either activation or repression domain, so that different targeted sequence of interest, e.g. different chromosomes of a genome, can be differentially regulated. In another embodiment, the nucleic acid-guided nuclease system protein can be dCpf1 fused different protein domain which can be recognized by different antibodies, so that different targeted sequence of interest, e.g. different DNA sequences within a sample mixture, can be differentially isolated.

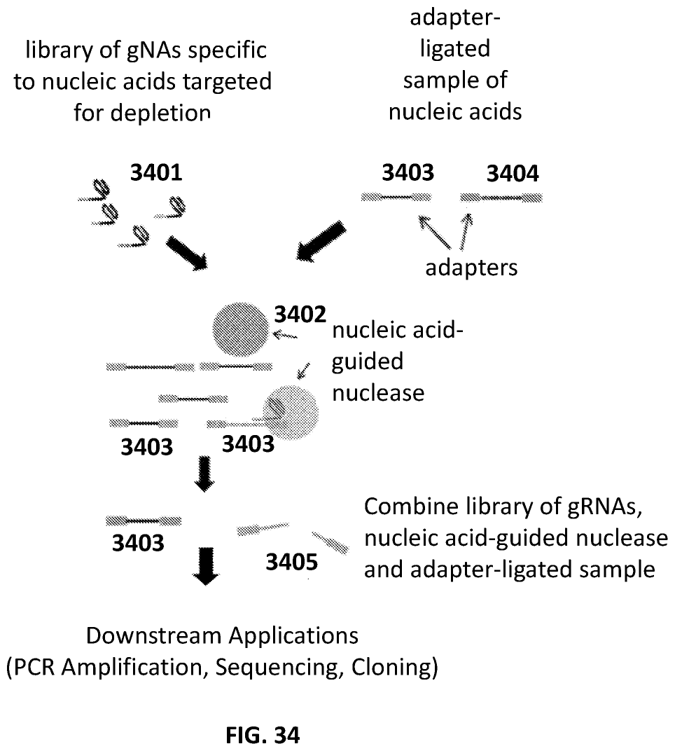

Exemplary methods of depleting nucleic acids targeted for depletion are depicted in FIG. 34. The methods depleting sequences targeted for depletion, thereby enriching for sequences of interest, can be combined with the ligation-free methods of preparing samples of nucleic acids described herein. A plurality of gNAs (3401) are used to target a nucleic acid-guided nuclease (3402) to nucleic acids targeted for depletion (3403) in a sample of adapter-ligated nucleic acids. The adapter ligated nucleic acids are generated by any of the methods of enrichment described herein that use modification-sensitive restriction enzymes to deplete nucleic acids targeted for depletion from a sample, either before or after an initial adapter ligation. In this method, the gNAs are specifically targeted to the nuclei acids targeted for depletion (3403), and not the nucleic acids of interest (3404), which are therefore not cut by the nucleic acid-guided nuclease (3402). Cleavage by the nucleic acid-guided nuclease results in nucleic acids targeted for depletion that are adapter ligated on one end (3405), and nucleic acids of interest that are adapter ligated on both ends (3403). These adapters can be used for downstream applications, for example adapter-mediated PCR amplification, sequencing (e.g. high throughput sequencing), quantification of the nucleic acids of interest in the sample and cloning.

In Vitro Transcription of gRNAs

In some embodiments, the gNAs comprise guide RNAs (gRNAs). In some embodiments of the methods of the invention, collections of gRNAs are made through the in vitro transcription of a DNA template. An exemplary DNA template of the disclosure comprises a first segment comprising a regulatory region; a second segment comprising a nucleic acid encoding a nucleic acid-guided nuclease (e.g., CRISPR/Cas) system protein-binding sequence; and a third segment encoding a targeting sequence. In some embodiments, the regulatory region comprises a T7, an SP6 or a T3 promoter.

In some embodiments, in particular those embodiments wherein the promoter is a T7 promoter, the T7 promoter comprises a sequence of 5′-TAATACGACTCACTATAGG-3′ (SEQ ID NO: 1). In some embodiments, the T7 promoter comprises a sequence of 5′-TAATACGACTCACTATAGGG-3′ (SEQ ID NO: 2). In some embodiments, the T7 promoter comprises a sequence of 5′-GCCTCGAGCTAATACGACTCACTATAGAG-3′ (SEQ ID NO: 3).

In some embodiments, the SP6 promoter comprises a sequence of 5′-ATTTAGGTGACACTATAG-3′ (SEQ ID NO: 4). In some embodiments, the SP6 promoter comprises a sequence of 5′-CATACGATTTAGGTGACACTATAG-3′ (SEQ ID NO: 5).

In some embodiments, the T3 promoter comprises a sequence of 5′ AATTAACCCTCACTAAAG 3′ (SEQ ID NO: 6).

In some embodiments, the gRNA DNA template is transcribed by a DNA dependent RNA polymerase. Polymerases of the disclosure can be RNA polymerase II or RNA polymerase III polymerases. In some embodiments, the polymerase is a T7 polymerase, an SP6 polymerase or a T3 polymerase. RNA polymerases of the disclosure may be wild type polymerases, artificial polymerases, or polymerases that have been optimized or engineered (e.g., for in vitro transcription). The activity of a polymerases of the disclosure may be highly specific for given promoter sequence (e.g., the T7 polymerase for the T7 promoter, the SP6 polymerase for the SP6 promoter, or the T3 polymerase for the T3 promoter).

The T7 promoter is recognized by and supports transcription by the T7 bacteriophage RNA polymerase. T7 polymerases of the disclosure may be wild type T7 polymerases, artificial T7 polymerases, or T7 polymerases that have been optimized or engineered (e.g., for in vitro transcription). The T7 polymerase is a DNA dependent RNA polymerase that catalyzes the formation of RNA from a DNA template in the 5′ to 3 direction. The DNA template may be double stranded or single stranded. T7 polymerase exhibits high specificity for the T7 promoter, can produce robust transcription in vitro, and is capable of incorporating modified nucleotides (e.g., labeled nucleotides) into nascent RNA transcripts. These features of the T7 polymerase make it an excellent polymerase for synthesizing gRNAs of the disclosure, e.g. the collections of gRNAs of the disclosure.

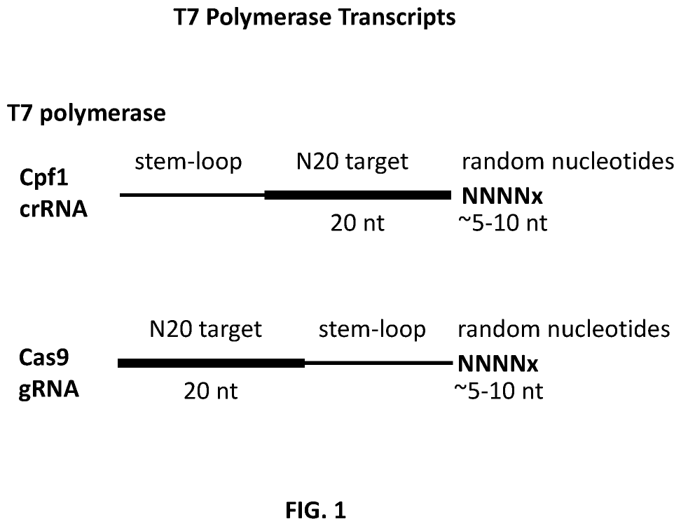

However, under some conditions, polymerases such as T7, T3 or SP6 polymerases add a few (e.g., 5-10) untemplated random nucleotides to the 3′ ends of in vitro transcribed RNA transcripts. For Cas9 system gRNAs, which are arranged 5′-recognition site-protein binding sequence stem loop sequence-3′, these untemplated nucleotides are added to the stem loop region, where there is less likely to be an impact on performance of the gRNA (see FIG. 1). For Cpf1 system gRNAs, which are arranged 5′-protein binding sequence stem loop sequence-recognition site-3′, the untemplated nucleotides are added to the recognition site region (see FIG. 1), which can affect gRNA performance. For example, a Cpf1 gRNA with untemplated nucleotides that match nucleotides adjacent to a sequence similar to the targeting sequence (aka, recognitions site) in a target genome (an “off target” sequence) could result in the mis-targeting of the Cpf1-gRNA complex to the off target sequence and not the target sequence. Previous work using Cpf1 (e.g. for gene editing) has employed other methods of gRNA generation, such as extension along a template, which would not produce extra nucleotides.

### Size Selection

Provided herein are methods for controlling the size of in vitro transcribed RNAs, for example gRNAs, through size selection techniques.

An RNA, e.g. a Cpf1 system protein compatible gRNA, can be in vitro transcribed from a template DNA comprising, from 5′ to 3: a first nucleic acid sequence encoding a promoter, a second nucleic acid sequence comprising a nucleic acid guided nuclease system protein binding sequence (e.g., a stem loop), a sequence encoding a targeting sequence and a sequence encoding a primer binding sequence. In some embodiments, the DNA dependent RNA polymerase comprises T7, SP6 or T3. In some embodiments, the DNA dependent RNA polymerase is T7. The transcribed RNA comprises, from 5′ to 3′, the sequence encoding the stem-loop, the sequence encoding the targeting sequence and the sequence encoding the primer binding sequence. In some embodiments, Cpf1 gRNAs are approximately 43 bases in length, comprising a 20-nucleotide targeting sequence and at least a 19 base pair nucleic acid guided nuclease system protein binding sequence (e.g. 19 bp, 20 bp, 21 bp, 22 bp, or 23 bp). Accordingly, in some embodiments, the size cut off for size-based separation of gRNAs is approximately 39, 40, 41, 42, 43, 44, or 45 base pairs. In some embodiments, Cpf1 gRNAs are approximately 38 bases in length, comprising a 15-nucleotide targeting sequence and at least a 19 base pair nucleic acid guided nuclease system protein binding sequence (e.g. 19 bp, 20 bp, 21 bp, 22 bp, or 23 bp). Accordingly, in some embodiments, the size cut off for size-based separation of gRNAs is approximately 34, 35, 36, 37, 38, 39, or 40 base pairs.

In some embodiments the targeting sequence is 15-250 bp. In some embodiments, the targeting sequence is greater than 14 bp, is greater than 15 bp, is greater than 16 bp, is greater than 17 bp, is greater than 18 bp, is greater than 19 bp, is greater than 20 bp, is greater than 21 bp, greater than 22 bp, greater than 23 bp, greater than 24 bp, greater than 25 bp, greater than 26 bp, greater than 27 bp, greater than 28 bp, greater than 29 bp, greater than 30 bp, greater than 40 bp, greater than 50 bp, greater than 60 bp, greater than 70 bp, greater than 80 bp, greater than 90 bp, greater than 100 bp, greater than 110 bp, greater than 120 bp, greater than 130 bp, greater than 140 bp, or even greater than 150 bp. In an exemplary embodiment, the targeting sequence is greater than 30 bp. In some embodiments, the targeting sequences of the present invention range in size from 30-50 bp. In some embodiments, targeting sequences of the present invention range in size from 30-75 bp. In some embodiments, targeting sequences of the present invention range in size from 30-100 bp. For example, a targeting sequence can be at least 14, 15 bp, 16 bp, 17 bp, 18 bp, 19 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, or 250 bp. In specific embodiments, the targeting sequence is at least 20 bp. In specific embodiments, the targeting sequence is 14-25 bp. In specific embodiments, the targeting sequence is 14, 15, 16, 17, 18, 19, 20, 21, 22, 23, 24 or 25 bp. In specific embodiments, the targeting sequence is 20 bp (an N20 targeting sequence).

The size cut off for size-based separation of gRNAs depends on the lengths of the targeting sequence and nucleic acid guided nuclease system protein binding sequence in a specific embodiment. In an exemplary embodiment, the size cut off is summed the length of the targeting sequence plus the length of the nucleic acid guided nuclease system protein binding sequence. The length of the nucleic acid guided nuclease system protein binding sequence can be, for example, 19-23 bp. In an exemplary embodiment, the size cut off is slightly larger than summed the length of the targeting sequence plus the length of the protein binding stem loop sequence. For example, the size cut off is 1, 2, 3, 4, 5, 10 or 15 bp longer than the length of the gNA. In an additional exemplary embodiment, the size cut off is a range that includes the summed length targeting sequence plus the length of the nucleic acid guided nuclease system protein binding sequence. For example, gRNAs that are shorter and longer than the summed length targeting sequence plus the length of the nucleic acid guided nuclease system protein binding sequence by 1, 2, 3, 4, 5, 10 or 15 bp can be included in the size cut off range.

In vitro transcribed RNAs can be size selected through standard size selection techniques. In vitro transcribed gRNAs can be size selected through standard size selection techniques. For example, gel electrophoresis can be used to pick the best sized guide RNAs. In vitro transcribed gRNAs can be run on a gel next to an RNA ladder, the region of the gel spanning the desired size range excised, and the gRNAs extracted. The gel can be a polyacrylamide gel, for example a 5% or 10% polyacrylamide gel. In some embodiments, the polyacrylamide gel is a denaturing polyacrylamide gel.

Alternatively, gRNAs can be size selected through size exclusion chromatography. In some embodiments, the size exclusion chromatography is gel-filtration chromatography.

### Removal of 3′ Nucleotides

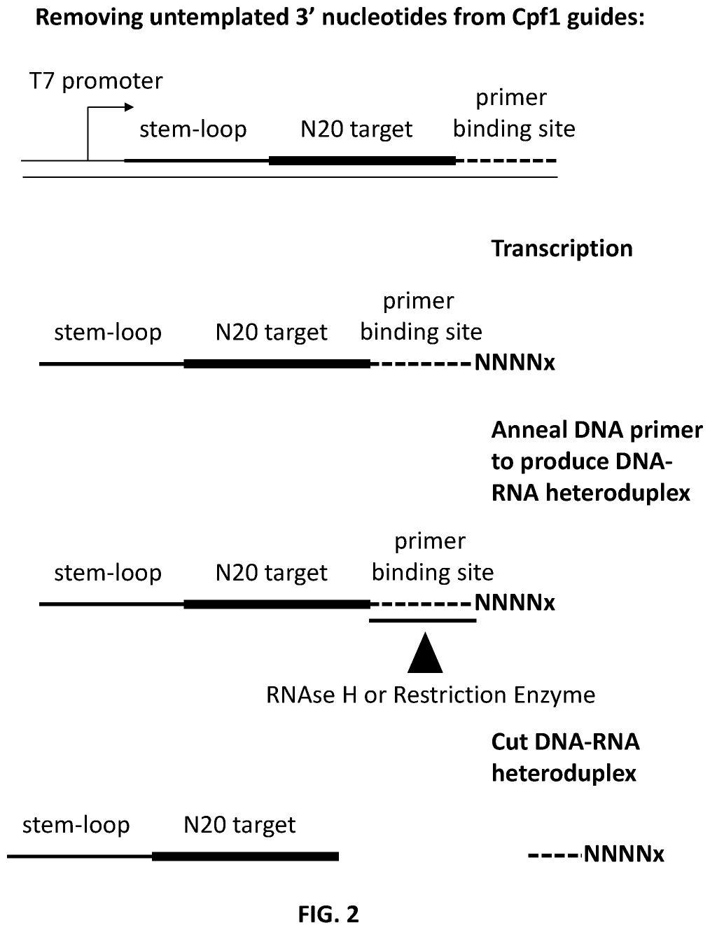

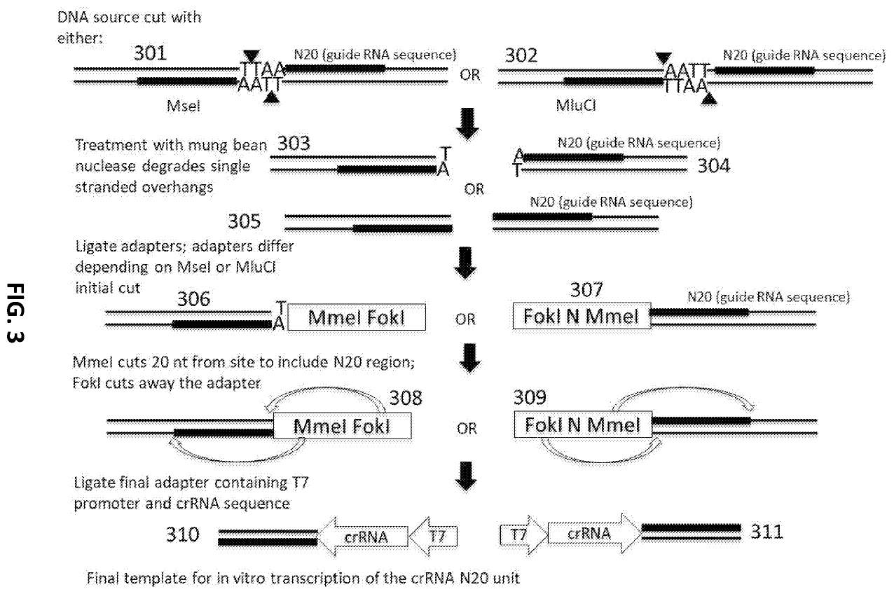

The invention provides methods for removing 3′ nucleotides from in vitro transcribed RNAs which are described below. Exemplary methods are shown in FIG. 2. An RNA, e.g. a Cpf1 system compatible gRNA, can be in vitro transcribed from a template DNA comprising from 5′ to 3: a first nucleic acid sequence encoding a promoter, a second nucleic acid sequence comprising a nucleic acid guided nuclease system protein binding sequence (e.g., a stem loop), a sequence encoding a targeting sequence and a sequence encoding a primer binding sequence. In some embodiments, the DNA dependent RNA polymerase comprises T7, SP6 or T3. In some embodiments, the DNA dependent RNA polymerase is a T7 polymerase. The transcribed RNA comprises, from 5′ to 3′, the sequence encoding the stem-loop, the sequence encoding the targeting sequence and the sequence encoding the primer binding sequence. A single stranded DNA (ssDNA) comprising a sequence complementary to the sequence encoding the primer binding sequence is hybridized to the primer binding sequence in the transcribed RNA, to form an RNA/DNA heteroduplex region.

In some embodiments, the RNA/DNA heteroduplex region of the in vitro transcribed RNA is digested with a Ribonuclease H (RNase H) enzyme. RNase H is a non-sequence specific endonuclease that catalyzes the cleavage of RNA in RNA/DNA heteroduplexes by hydrolyzing the phosphodiester bonds of the RNA when it is hybridized to DNA. RNase H enzymes of the disclosure may be wild type, recombinant, or engineered (e.g., for in vitro functionality). An exemplary RNase H is available from NEB (catalog #M0297S).

In some embodiments, the primer binding sequence comprises a recognition site for a restriction enzyme. A single stranded DNA (ssDNA) comprising a sequence complementary to the sequence encoding the primer binding sequence is hybridized to the primer binding sequence in the transcribed RNA, to form an RNA/DNA heteroduplex region. Following hybridization of a single stranded DNA to the primer binding sequence of the in vitro transcribed RNA, the RNA/DNA heteroduplex region is cut with a restriction enzyme. In some embodiments, the restriction enzyme is a Type II restriction enzyme, for example a Type IIP restriction enzyme. In some embodiments, the Type IIP restriction enzyme is selected from the group consisting of AvaII, AvrII, HaeIII, HinfI or TaqI. In some embodiments, the restriction enzyme comprises SaII, HhaI, AluI, HindIII, EcoRI or MspI. Restriction enzymes that hydrolyze RNA in RNA/DNA heteroduplexes are described in Murray et al. Nucleic Acids Res (2010), 38:8257-8268, the contents of which are hereby incorporated by reference in their entirety.

In some embodiments, the DNA template is a synthetic DNA. For example, a collection of synthetic DNA fragments designed and synthesized via the methods of the disclosure. In some embodiments, the DNA is a PCR amplification product. For example, the DNA may be a PCR amplification product of a collection of DNA gRNA templates produced from a starting DNA sample using the methods of the disclosure. In some embodiments, the DNA may be a plasmid. Plasmids can be linearized with restriction enzymes, for example, a type II restriction endonuclease, before in vitro transcription of the corresponding RNA.

Guide Nucleic Acids (gNAs)

Provided herein are guide nucleic acids (gNAs) and collections of gNAs derivable from any nucleic acid source. In some embodiments, the gNAs comprise guide ribonucleic acids (gRNAs). In some embodiments, the gNAs comprise deoxyribonucleic acids (gDNAs). In some embodiments, the gNAs comprise RNA and DNA.

In some embodiments, the collection of gNAs comprises or consists essentially of gRNAs. In some embodiments, the collection of gNAs comprises or consists essentially of gDNAs. In some embodiments, the collection of gNAs comprises gRNAs and gDNAs.

The gNAs (e.g., gRNAs and gDNAs) and collections of gNAs provided herein are useful for a variety of applications, including targeting sequences for depletion, partitioning, capture, or enrichment of target sequences of interest; genome-wide labeling; genome-wide editing; genome-wide function screens; and genome-wide regulation.

Guide Ribonucleic Acids (gRNAs)

Provided herein are guide ribonucleic acids (gRNAs) derivable from any nucleic acid source, which do not contain additional untemplated 3′ nucleotides. The nucleic acid source can be DNA or RNA. Provided herein are methods to generate gRNAs from any source nucleic acid, including DNA from a single organism, or mixtures of DNA from multiple organisms, or mixtures of DNA from multiple species, or DNA from clinical samples, or DNA from forensic samples, or DNA from environmental samples, or DNA from metagenomic DNA samples (for example a sample that contains more than one species of organism). Examples of any source DNA include, but are not limited to any genome, any genome fragment, cDNA, synthetic DNA, or a DNA collection (e.g. a SNP collection, DNA libraries). The gRNAs provided herein can be used for genome-wide applications.

gRNAs that are in vitro transcribed from a corresponding DNA template derived from a nucleic acid source can contain additional untemplated nucleotides at the 3′ end of the gRNA. For Cpf1 system protein compatible gRNAs, the arrangement of the nucleic acid guided nuclease system protein-binding sequence relative the targeting sequence makes these additional nucleotides that result from in vitro transcription steps potentially problematic. Provided herein are methods and compositions to remove additional 3′ nucleotides from gRNAs to generate gRNAs and collections of gRNAs with 3′ ends that do not contain additional untemplated 3′ nucleotides. These methods or removing 3′ nucleotides increase the sequence identity between the gRNA or collection of gRNAs and the nucleic acid source from which the gRNA or collection of gRNAs was derived. In some embodiments, this increases the fidelity of the protein-gRNA complex to a target site of interest.

In some embodiments, the gRNAs are derived from genomic sequences (e.g., genomic DNA). In some embodiments, the gRNAs are derived from mammalian genomic sequences. In some embodiments, the gRNAs are derived from eukaryotic genomic sequences. In some embodiments, the gRNAs are derived from prokaryotic genomic sequences. In some embodiments, the gRNAs are derived from viral genomic sequences. In some embodiments, the gRNAs are derived from bacterial genomic sequences. In some embodiments, the gRNAs are derived from plant genomic sequences. In some embodiments, the gRNAs are derived from microbial genomic sequences. In some embodiments, the gRNAs are derived from genomic sequences from a parasite, for example a eukaryotic parasite.

In some embodiments, the gRNAs are derived from repetitive DNA. In some embodiments, the gRNAs are derived from abundant DNA. In some embodiments, the gRNAs are derived from mitochondrial DNA. In some embodiments, the gRNAs are derived from ribosomal DNA. In some embodiments, the gRNAs are derived from centromeric DNA. In some embodiments, the gRNAs are derived from DNA comprising Alu elements (Alu DNA). In some embodiments, the gRNAs are derived from DNA comprising long interspersed nuclear elements (LINE DNA). In some embodiments, the gRNAs are derived from DNA comprising short interspersed nuclear elements (SINE DNA). In some embodiments, the abundant DNA comprises ribosomal DNA. In some embodiments, the abundant DNA comprises host DNA (e.g., host genomic DNA or all host DNA). In an example, the gRNAs can be derived from host DNA (e.g., human, animal, plant) for the depletion of host DNA to allow for easier analysis of other DNA that is present (e.g., bacterial, viral, or other metagenomic DNA). In another example, the gRNAs can be derived from the one or more most abundant types (e.g., species) in a mixed sample, such as the one or more most abundant bacteria species in a metagenomic sample. The one or more most abundant types (e.g., species) can comprise the two, three, four, five, six, seven, eight, nine, ten, or more than ten most abundant types (e.g., species). The most abundant types can be the most abundant kingdoms, phyla or divisions, classes, orders, families, genuses, species, or other classifications. The most abundant types can be the most abundant cell types, such as epithelial cells, bone cells, muscle cells, blood cells, adipose cells, or other cell types. The most abundant types can be non-cancerous cells. The most abundant types can be cancerous cells. The most abundant types can be animal, human, plant, fungal, bacterial, or viral. gRNAs can be derived from both a host and the one or more most abundant non-host types (e.g., species) in a sample, such as from both human DNA and the DNA of the one or more most abundant bacterial species. In some embodiments, the abundant DNA comprises DNA from the more abundant or most abundant cells in a sample. For example, for a specific sample, the highly abundant cells can be extracted and their DNA can be used to produce gRNAs; these gRNAs can be used to produce depletion library and applied to original sample to enable or enhance sequencing or detection of low abundance targets.

In some embodiments, the gRNAs are derived from DNA comprising short terminal repeats (STRs).

In some embodiments, the gRNAs are derived from DNA sequences with low or no variation across human populations.

In some embodiments, the gRNAs are derived from a genomic fragment, comprising a region of the genome, or the whole genome itself. In one embodiment, the genome is a DNA genome. In another embodiment, the genome is an RNA genome.

In some embodiments, the gRNAs are derived from a eukaryotic or prokaryotic organism; from a mammalian organism or a non-mammalian organism; from an animal or a plant; from a bacteria or virus; from an animal parasite; from a pathogen.

In some embodiments, the gRNAs are derived from any mammalian organism. In one embodiment the mammal is a human. In another embodiment the mammal is a livestock animal, for example a horse, a sheep, a cow, a pig, or a donkey. In another embodiment, a mammalian organism is a domestic pet, for example a cat, a dog, a gerbil, a mouse, a rat. In another embodiment the mammal is a type of a monkey.

In some embodiments, the gRNAs are derived from any bird or avian organism. An avian organism includes but is not limited to chicken, turkey, duck and goose.

In some embodiments, the sequences of interest are from an insect. Insects include, but are not limited to honeybees, solitary bees, ants, flies, wasps or mosquitoes.

In some embodiments, the gRNAs are derived from a plant. In one embodiment, the plant is rice, maize, wheat, rose, grape, coffee, fruit, tomato, potato, or cotton.

In some embodiments, the gRNAs are derived from a species of bacteria. In one embodiment, the bacteria are tuberculosis-causing bacteria.

In some embodiments, the gRNAs are derived from a virus.

In some embodiments, the gRNAs are derived from a species of fungi.

In some embodiments, the gRNAs are derived from a species of algae.

In some embodiments, the gRNAs are derived from any mammalian parasite.

In some embodiments, the gRNAs are derived from any mammalian parasite. In one embodiment, the parasite is a worm. In another embodiment, the parasite is a malaria-causing parasite. In another embodiment, the parasite is a Leishmaniosis-causing parasite. In another embodiment, the parasite is an amoeba.

In some embodiments, the gRNAs are derived from a nucleic acid target. Contemplated targets include, but are not limited to, pathogens; single nucleotide polymorphisms (SNPs), insertions, deletions, tandem repeats, or translocations; human SNPs or STRs; potential toxins; or animals, fungi, and plants. In some embodiments, the gRNAs are derived from pathogens, and are pathogen-specific gRNAs.

In some embodiments, a gRNA of the invention comprises a first nucleic acid segment comprising a nucleic acid guided nuclease system (e.g., CRISPR/Cas system) protein-binding sequence (e.g., a stem loop sequence) and a second nucleic acid segment comprising a targeting sequence, wherein the targeting sequence is 15-250 bp. In some embodiments, the targeting sequence is greater than 14 bp, is greater than 15 bp, is greater than 16 bp, is greater than 17 bp, is greater than 18 bp, is greater than 19 bp, is greater than 20 bp, the targeting sequence is greater than 21 bp, greater than 22 bp, greater than 23 bp, greater than 24 bp, greater than 25 bp, greater than 26 bp, greater than 27 bp, greater than 28 bp, greater than 29 bp, greater than 30 bp, greater than 40 bp, greater than 50 bp, greater than 60 bp, greater than 70 bp, greater than 80 bp, greater than 90 bp, greater than 100 bp, greater than 110 bp, greater than 120 bp, greater than 130 bp, greater than 140 bp, or even greater than 150 bp. In an exemplary embodiment, the targeting sequence is greater than 30 bp. In some embodiments, the targeting sequences of the present invention range in size from 30-50 bp. In some embodiments, targeting sequences of the present invention range in size from 30-75 bp. In some embodiments, targeting sequences of the present invention range in size from 30-100 bp. For example, a targeting sequence can be at least 14 bp, 15 bp, 16 bp, 17 bp, 18 bp, 19 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, or 250 bp. In specific embodiments, the targeting sequence is at least 20 bp. In specific embodiments, the targeting sequence is 14-25 bp. In specific embodiments, the targeting sequence is 14, 15, 16, 17, 18, 19, 20, 21, 22, 23, 24 or 25 bp. In specific embodiments, the targeting sequence is 20 bp (an N20 targeting sequence). In some cases, methods of the present disclosure are presented with reference to generating gRNAs with 20-basepair targeting sequences; these methods can be modified to yield targeting sequences with other lengths, for example by adjusting the spacing between a restriction enzyme site and the targeting sequence such that the restriction enzyme cuts to yield a different length targeting sequence.

In some embodiments, target-specific gRNAs can comprise a nucleic acid sequence that is complementary to a region on the opposite strand of the targeted nucleic acid sequence 3′ to a PAM sequence, which can be recognized by a nucleic acid-guided nuclease system (e.g., CRISPR/Cas system) protein. In some embodiments the targeted nucleic acid sequence is immediately 3′ to a PAM sequence. In specific embodiments, the nucleic acid sequence of the gRNA that is complementary to a region in a target nucleic acid is 15-250 bp. In specific embodiments, the nucleic acid sequence of the gRNA that is complementary to a region in a target nucleic acid is 20, 21, 22, 23, 24, 25, 30, 35, 40, 45, 50, 60, 70, 75, 80, 90, or 100 bp.

In some embodiments, the gRNAs comprise any purines or pyrimidines (and/or modified versions of the same). In some embodiments, the gRNAs comprise adenine, uracil, guanine, and cytosine (and/or modified versions of the same). In some embodiments, the gRNAs comprise adenine, thymine, guanine, and cytosine (and/or modified versions of the same). In some embodiments, the gRNAs comprise adenine, thymine, guanine, cytosine and uracil (and/or modified versions of the same).

In some embodiments, the gRNAs comprise a label, are attached to a label, or are capable of being labeled. In some embodiments, the gRNA comprises a moiety that is further capable of being attached to a label. A label includes, but is not limited to, an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group (capable of undergoing a redox reaction), an aptamer, one member of a binding pair, a member of a FRET pair (either a donor or acceptor fluorophore), and combinations thereof.

In some embodiments, the gRNAs are attached to a substrate. The substrate can be made of glass, plastic, silicon, silica-based materials, functionalized polystyrene, functionalized polyethylene glycol, functionalized organic polymers, nitrocellulose or nylon membranes, paper, cotton, and materials suitable for synthesis. Substrates need not be flat. In some embodiments, the substrate is a 2-dimensional array. In some embodiments, the 2-dimensional array is flat. In some embodiments, the 2-dimensional array is not flat, for example, the array is a wave-like array. Substrates include any type of shape including spherical shapes (e.g., beads). Materials attached to substrates may be attached to any portion of the substrates (e.g., may be attached to an interior portion of a porous substrates material). In some embodiments, the substrate is a 3-dimensional array, for example, a microsphere. In some embodiments, the microsphere is magnetic. In some embodiments, the microsphere is glass. In some embodiments, the microsphere is made of polystyrene. In some embodiments, the microsphere is silica-based. In some embodiments, the substrate is an array with interior surface, for example, is a straw, tube, capillary, cylindrical, or microfluidic chamber array. In some embodiments, the substrate comprises multiple straws, capillaries, tubes, cylinders, or chambers.

Nucleic Acids Encoding gNAs

Also provided herein are nucleic acids encoding for gNAs.

In some embodiments, by encoding it is meant that a gDNA results from replication of a DNA encoding the gDNA, or that the nucleic acid is a DNA encoding the gDNA.

In some embodiments, by encoding it is meant that a gRNA results from the transcription of a nucleic acid encoding for a gRNA. T7 promoters are discussed in this disclosure, though the use of other appropriate promoters such as SP6 and T7 is also contemplated. In some embodiments, by encoding, it is meant that the nucleic acid is a template for the transcription of a gRNA. In some embodiments, by encoding, it is meant that a gRNA results from the reverse transcription of a nucleic acid encoding for a gRNA. In some embodiments, by encoding, it is meant that the nucleic acid is a template for the reverse transcription of a gRNA. In some embodiments, by encoding, it is meant that a gRNA results from the amplification of a nucleic acid encoding for a gRNA. In some embodiments, by encoding, it is meant that the nucleic acid is a template for the amplification of a gRNA.

In some embodiments the nucleic acid encoding for a gRNA comprises a first segment comprising a regulatory region; a second segment comprising a nucleic acid encoding a nucleic acid-guided nuclease system (e.g., CRISPR/Cas system) protein-binding sequence (e.g., a stem loop sequence); and a third segment comprising targeting sequence, wherein the third segment can range from 15 bp-250 bp.

In some embodiments, the nucleic acids encoding for gRNAs comprise DNA. In some embodiments, the first segment is double stranded DNA. In some embodiments, the first segment is single stranded DNA. In some embodiments, the second segment is single stranded DNA. In some embodiments, the third segment is single stranded DNA. In some embodiments, the second segment is double stranded DNA. In some embodiments, the third segment is double stranded DNA.

In some embodiments, the nucleic acids encoding for gRNAs comprise RNA.

In some embodiments the nucleic acids encoding for gRNAs comprise DNA and RNA.

In some embodiments, the regulatory region is a region capable of binding a transcription factor. In some embodiments, the regulatory region comprises a promoter. In some embodiments, the promoter is selected from the group consisting of T7, SP6, and T3. In some embodiments, in particular those embodiments wherein the promoter is a T7 promoter, the T7 promoter comprises a sequence of 5′-TAATACGACTCACTATAGG-3′ (SEQ ID NO: 1). In some embodiments, the T7 promoter comprises a sequence of 5′-TAATACGACTCACTATAGGG-3′ (SEQ ID NO: 2). In some embodiments, the T7 promoter comprises the sequence of (5′-GCCTCGAGCTAATACGACTCACTATAGAG-3′ (SEQ ID NO: 3). In some embodiments, the SP6 promoter comprises a sequence of 5′-ATTTAGGTGACACTATAG-3′ (SEQ ID NO: 4). In some embodiments, the SP6 promoter comprises a sequence of 5′-CATACGATTTAGGTGACACTATAG-3′ (SEQ ID NO: 5). In some embodiments, the T3 promoter comprises a sequence of 5′ AATTAACCCTCACTAAAG 3′ (SEQ ID NO: 6).

Collections of gRNAs not Containing 3′ Untemplated Nucleotides

Provided herein are collections (interchangeably referred to as libraries) of gRNAs.

Collections of gRNAs that are in vitro transcribed from a corresponding DNA template using a polymerase such as T7, SP6 or T3 can contain additional untemplated nucleotides at the 3′ end of the gRNA. For Cpf1 system protein compatible gRNAs, the arrangement of the nucleic acid guided nuclease system protein-binding sequence relative the targeting sequence makes these additional nucleotides potentially problematic. Provided herein are methods and compositions to remove additional 3′ nucleotides from gRNAs to generate gRNAs and collections of gRNAs with homogenous 3′ ends that do not contain additional untemplated 3′ nucleotides. These methods or removing 3′ nucleotides increase the sequence identity between the gRNA or collection of gRNAs and the nucleic acid source from which the gRNA or collection of gRNAs was derived.

As used herein, a collection of gRNAs denotes a mixture of gRNAs containing at least 10² unique gRNAs. In some embodiments a collection of gRNAs contains at least 10², at least 10³, at least 10⁴, at least 10⁵, at least 10⁶, at least 10⁷, at least 10⁸, at least 10⁹, at least 10¹⁰ unique gRNAs. In some embodiments a collection of gRNAs contains a total of at least 10², at least 10³, at least 10⁴, at least 10⁵, at least 10⁶, at least 10⁷, at least 10⁸, at least 10⁹, at least 10¹⁰ gRNAs.

In some embodiments, a collection of gRNAs comprises a first nucleic acid (NA) segment comprising a nucleic acid-guided nuclease system (e.g., CRISPR/Cas system) protein-binding sequence and a second NA segment comprising a targeting sequence, wherein at least 10% of the gRNAs in the collection vary in size. In some embodiments, the first and second segments are in 5′- to 3′-order′. In some embodiments, the first and second segments are in 3′- to 5′-order′.

In some embodiments, the size of the second segment varies from 15-250 bp, or 30-100 bp, or 22-30 bp, or 15-50 bp, or 15-25 bp, or 15-75 bp, or 15-100 bp, or 15-125 bp, or 15-150 bp, or 15-175 bp, or 15-200 bp, or 15-225 bp, or 15-250 bp, or 22-50 bp, or 22-75 bp, or 22-100 bp, or 22-125 bp, or 22-150 bp, or 22-175 bp, or 22-200 bp, or 22-225 bp, or 22-250 bp across the collection of gRNAs.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the second segments in the collection are greater than or equal to 15 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the second segments in the collection are greater than or equal to 20 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the second segments in the collection are greater than 21 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the second segments in the collection are greater than 25 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the second segments in the collection are greater than 30 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the second segments in the collection are 15-50 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the second segments in the collection are 30-100 bp.

In some particular embodiments, the size of the second segment is not 20 bp.

In some particular embodiments, the size of the second segment is not 21 bp.

In some embodiments, the targeting sequences of the gRNAs in the collection of gRNAs comprise unique 5′ ends. In some embodiments, the collection of gRNAs exhibit variability in sequence of the 5′ end of the targeting sequence, across the members of the collection. In some embodiments, the collection of gRNAs exhibit variability at least 5%, or at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75% variability in the sequence of the 5′ end of the targeting sequence, across the members of the collection.

In some embodiments, the 3′ end of the gRNA targeting sequence can be any purine or pyrimidine (and/or modified versions of the same). In some embodiments, the 3′ end of the gRNA targeting sequence is an adenine. In some embodiments, the 3′ end of the gRNA targeting sequence is a guanine. In some embodiments, the 3′ end of the gRNA targeting sequence is a cytosine. In some embodiments, the 3′ end of the gRNA targeting sequence is a uracil. In some embodiments, the 3′ end of the gRNA targeting sequence is a thymine. In some embodiments, the 3′ end of the gRNA targeting sequence is not cytosine.

In some embodiments, the collection of gRNAs comprises targeting sequences which can base-pair with the targeted DNA, wherein the target of interest is spaced at least every 1 bp, at least every 2 bp, at least every 3 bp, at least every 4 bp, at least every 5 bp, at least every 6 bp, at least every 7 bp, at least every 8 bp, at least every 9 bp, at least every 10 bp, at least every 11 bp, at least every 12 bp, at least every 13 bp, at least every 14 bp, at least every 15 bp, at least every 16 bp, at least every 17 bp, at least every 18 bp, at least every 19 bp, 20 bp, at least every 25 bp, at least every 30 bp, at least every 40 bp, at least every 50 bp, at least every 100 bp, at least every 200 bp, at least every 300 bp, at least every 400 bp, at least every 500 bp, at least every 600 bp, at least every 700 bp, at least every 800 bp, at least every 900 bp, at least every 1000 bp, at least every 2500 bp, at least every 5000 bp, at least every 10,000 bp, at least every 15,000 bp, at least every 20,000 bp, at least every 25,000 bp, at least every 50,000 bp, at least every 100,000 bp, at least every 250,000 bp, at least every 500,000 bp, at least every 750,000 bp, or even at least every 1,000,000 bp across a genome of interest.

In some embodiments, the collection of gRNAs comprises a first NA segment comprising a nucleic acid-guided nuclease system (e.g., CRISPR/Cas system) protein-binding sequence, and a second NA segment comprising a targeting sequence; wherein the gRNAs in the collection can have a variety of first NA segments with various specificities for protein members of the nucleic acid-guided nuclease system (e.g., CRISPR/Cas system). For example a collection of gRNAs as provided herein, can comprise members whose first segment comprises a nucleic acid-guided nuclease system (e.g., CRISPR/Cas system) protein-binding sequence specific for a first nucleic acid-guided nuclease system (e.g., CRISPR/Cas system) protein; and also comprises members whose first segment comprises a nucleic acid-guided nuclease system (e.g., CRISPR/Cas system) protein-binding sequence specific for a second nucleic acid-guided nuclease system (e.g., CRISPR/Cas system) protein, wherein the first and second nucleic acid-guided nuclease system (e.g., CRISPR/Cas system) proteins are not the same. In some embodiments a collection of gRNAs as provided herein comprises members that exhibit specificity to at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or even at least 20 nucleic acid-guided nuclease system (e.g., CRISPR/Cas system) proteins. In one specific embodiment, a collection of gRNAs as provided herein comprises members that exhibit specificity for a Cpf1 protein and another protein selected from the group consisting of Cas9, Cas3, Cas8a-c, Cas10, Cse1, Csy1, Csn2, Cas4, Csm2, Cm5, CasX, Cas13, Cas14 and CasY. In some embodiments, the nucleic acid-guided nuclease system protein-binding sequences specific for the first and second nucleic acid-guided nuclease system proteins are both 5′ of the second NA segment comprising a targeting sequence. In some embodiments, the nucleic acid-guided nuclease system protein-binding sequences specific for the first and second nucleic acid-guided nuclease system proteins are both 3′ of the second NA segment comprising a targeting sequence. In some embodiments, the nucleic acid-guided nuclease system protein-binding sequence specific for the first nucleic acid-guided nuclease system (e.g., CRISPR/Cas system) protein is 5′ of the second NA segment comprising a targeting sequence and the second nucleic acid-guided nuclease system protein-binding sequences specific for the second nucleic acid-guided nuclease system protein is 3′ of the second NA segment comprising a targeting sequence. The order of the second NA segment comprising a targeting sequence and the first NA segment comprising a nucleic acid-guided nuclease system protein-binding sequence will depend on the nucleic acid-guided nuclease system protein. The appropriate 5′ to 3′ arrangement of the first and second NA segments and choice of nucleic acid-guided nuclease system proteins will be apparent to one of ordinary skill in the art.

In some embodiments, a plurality of the gRNA members of the collection are attached to a label, comprise a label or are capable of being labeled. In some embodiments, the gRNA comprises a moiety that is further capable of being attached to a label. Exemplary but non-limiting moieties comprise digoxigenin (DIG) and fluorescein (FITC). A label includes, but is not limited to, enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group (capable of undergoing a redox reaction), an aptamer, one member of a binding pair, a member of a FRET pair (either a donor or acceptor fluorophore), and combinations thereof.

In some embodiments, a plurality of the gRNA members of the collection are attached to a substrate. The substrate can be made of glass, plastic, silicon, silica-based materials, functionalized polystyrene, functionalized polyethylene glycol, functionalized organic polymers, nitrocellulose or nylon membranes, paper, cotton, and materials suitable for synthesis. Substrates need not be flat. In some embodiments, the substrate is a 2-dimensional array. In some embodiments, the 2-dimensional array is flat. In some embodiments, the 2-dimensional array is not flat, for example, the array is a wave-like array. Substrates include any type of shape including spherical shapes (e.g., beads). Materials attached to substrates may be attached to any portion of the substrates (e.g., may be attached to an interior portion of a porous substrates material). In some embodiments, the substrate is a 3-dimensional array, for example, a microsphere. In some embodiments, the microsphere is magnetic. In some embodiments, the microsphere is glass. In some embodiments, the microsphere is made of polystyrene. In some embodiments, the microsphere is silica-based. In some embodiments, the substrate is an array with interior surface, for example, is a straw, tube, capillary, cylindrical, or microfluidic chamber array. In some embodiments, the substrate comprises multiple straws, capillaries, tubes, cylinders, or chambers.

Collections of Nucleic Acids Encoding gRNAs

Provided herein are collections (interchangeably referred to as libraries) of nucleic acids encoding for gNAs. In some embodiments, the gNAs are gDNAs, gRNAs or a combination thereof. In some embodiments, the gNAs are gRNAs.

In some embodiments, gRNAs in the collections of gRNAs do not contain untemplated 3′ nucleotides. In some embodiments, by encoding it is meant that a gRNA results from the transcription of a nucleic acid encoding for a gRNA. In some embodiments, by encoding, it is meant that the nucleic acid is a template for the transcription of a gRNA.

As used herein, a collection of nucleic acids encoding for gNAs denotes a mixture of nucleic acids containing at least 10² unique nucleic acids. In some embodiments a collection of nucleic acids encoding for gRNAs contains at least 10², at least 10³, at least 10⁴, at least 10⁵, at least 10⁶, at least 10⁷, at least 10⁸, at least 10⁹, at least 10¹⁰ unique nucleic acids encoding for gNAs. In some embodiments a collection of nucleic acids encoding for gNAs contains a total of at least 10², at least 10³, at least 10⁴, at least 10⁵, at least 10⁶, at least 10⁷, at least 10⁸, at least 10⁹, at least 10¹⁰ nucleic acids encoding for gNAs.

In some embodiments, a collection of nucleic acids encoding for gNAs comprises a first segment comprising a regulatory region; a second segment comprising a nucleic acid encoding a nucleic acid-guided nuclease system (e.g., CRISPR/Cas system) protein-binding sequence; and a third segment comprising a targeting sequence; wherein at least 10% of the nucleic acids in the collection vary in size.

In some embodiments, the first, second, and third segments are in 5′- to 3′-order′.

In some embodiments, the first, second and third segments are arranged, from 5′ to 3′, first segment, third segment, and second segment.

In some embodiments, the nucleic acids encoding for gNAs comprise DNA. In some embodiments, the first segment is single stranded DNA. In some embodiments, the first segment is double stranded DNA. In some embodiments, the second segment is single stranded DNA. In some embodiments, the third segment is single stranded DNA. In some embodiments, the second segment is double stranded DNA. In some embodiments, the third segment is double stranded DNA.

In some embodiments, the nucleic acids encoding for gNAs comprise RNA.

In some embodiments the nucleic acids encoding for gNAs comprise DNA and RNA.

In some embodiments, the regulatory region is a region capable of binding a transcription factor. In some embodiments, the regulatory region comprises a promoter. In some embodiments, the promoter is selected from the group consisting of T7, SP6, and T3. In some embodiments, in particular those embodiments wherein the promoter is a T7 promoter, the T7 promoter comprises a sequence of 5′-TAATACGACTCACTATAGG-3′ (SEQ ID NO: 1). In some embodiments, the T7 promoter comprises a sequence of 5′-TAATACGACTCACTATAGGG-3′ (SEQ ID NO: 2). In some embodiments, the T7 promoter comprises a sequence of 5′-GCCTCGAGCTAATACGACTCACTATAGAG-3′ (SEQ ID NO: 3). In some embodiments, the SP6 promoter comprises a sequence of 5′-ATTTAGGTGACACTATAG-3′ (SEQ ID NO: 4). In some embodiments, the SP6 promoter comprises a sequence of 5′-CATACGATTTAGGTGACACTATAG-3′ (SEQ ID NO: 5). In some embodiments, the T3 promoter comprises a sequence of 5′ AATTAACCCTCACTAAAG 3′ (SEQ ID NO: 6).

In some embodiments, the size of the third segments (targeting sequence) in the collection varies from 15-250 bp, or 30-100 bp, or 22-30 bp, or 15-50 bp, or 15-25 bp, or 15-75 bp, or 15-100 bp, or 15-125 bp, or 15-150 bp, or 15-175 bp, or 15-200 bp, or 15-225 bp, or 15-250 bp, or 22-50 bp, or 22-75 bp, or 22-100 bp, or 22-125 bp, or 22-150 bp, or 22-175 bp, or 22-200 bp, or 22-225 bp, or 22-250 bp across the collection of gNAs.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the third segments in the collection are greater than or equal to 15 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the third segments in the collection are greater than or equal to 20 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the third segments in the collection are greater than 21 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the third segments in the collection are greater than 25 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the third segments in the collection are greater than 30 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the third segments in the collection are 15-50 bp.

In some embodiments, at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100% of the third segments in the collection are 30-100 bp.

In some particular embodiments, the size of the third segment is not 20 bp.

In some particular embodiments, the size of the third segment is not 21 bp.

In some embodiments, the targeting sequence of the gNAs in the collection of gNAs comprise unique 5′ ends. In some embodiments, the collection of gRNAs exhibit variability in sequence of the 5′ end of the targeting sequence, across the members of the collection. In some embodiments, the collection of gNAs exhibit variability at least 5%, or at least 10%, or at least 15%, or at last 20%, or at least 25%, or at least 30%, or at least 35%, or at least 40%, or at least 45%, or at least 50%, or at least 55%, or at least 60%, or at least 65%, or at least 70%, or at least 75% variability in the sequence of the 5′ end of the targeting sequence, across the members of the collection.

In some embodiments, the collection of nucleic acids comprises targeting sequences, wherein the target of interest is spaced at least every 1 bp, at least every 2 bp, at least every 3 bp, at least every 4 bp, at least every 5 bp, at least every 6 bp, at least every 7 bp, at least every 8 bp, at least every 9 bp, at least every 10 bp, at least every 11 bp, at least every 12 bp, at least every 13 bp, at least every 14 bp, at least every 15 bp, at least every 16 bp, at least every 17 bp, at least every 18 bp, at least every 19 bp, 20 bp, at least every 25 bp, at least every 30 bp, at least every 40 bp, at least every 50 bp, at least every 100 bp, at least every 200 bp, at least every 300 bp, at least every 400 bp, at least every 500 bp, at least every 600 bp, at least every 700 bp, at least every 800 bp, at least every 900 bp, at least every 1000 bp, at least every 2500 bp, at least every 5000 bp, at least every 10,000 bp, at least every 15,000 bp, at least every 20,000 bp, at least every 25,000 bp, at least every 50,000 bp, at least every 100,000 bp, at least every 250,000 bp, at least every 500,000 bp, at least every 750,000 bp, or even at least every 1,000,000 bp across a genome of interest.

In some embodiments, the collection of nucleic acids encoding for gNAs comprise a second segment encoding for a nucleic acid-guided nuclease system (e.g., CRISPR/Cas system) protein-binding sequence, wherein the segments in the collection vary in their specificity for protein members of the nucleic acid-guided nuclease system (e.g., CRISPR/Cas system). For example, a collection of nucleic acids encoding for gNAs as provided herein, can comprise members whose second segment encode for a nucleic acid-guided nuclease system (e.g., CRISPR/Cas system) protein-binding sequence specific for a first nucleic acid-guided nuclease system (e.g., CRISPR/Cas system) protein; and also comprises members whose second segment encodes for a nucleic acid-guided nuclease system (e.g., CRISPR/Cas system) protein-binding sequence specific for a second nucleic acid-guided nuclease system (e.g., CRISPR/Cas system) protein, wherein the first and second nucleic acid-guided nuclease system (e.g., CRISPR/Cas system) proteins are not the same.

In some embodiments, a collection of nucleic acids encoding for gNAs as provided herein comprises members that exhibit specificity to at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or even at least 20 nucleic acid-guided nuclease system (e.g., CRISPR/Cas system) proteins. In one specific embodiment, a collection of nucleic acids encoding for gRNAs as provided herein comprises members that exhibit specificity for a Cpf1 protein and another protein selected from the group consisting of Cas9, Cas3, Cas8a-c, Cas10, Cse1, Csy1, Csn2, Cas4, Csm2, CasX, CasY, Cas13, Cas14 and Cm5. In one specific embodiment, a collection of nucleic acids encoding for gRNAs as provided herein comprises members that exhibit specificity for a Cas9 protein and another protein selected from the group consisting of Cpf1, Cas3, Cas8a-c, Cas10, Cse1, Csy1, Csn2, Cas4, Csm2, CasX, CasY, Cas13, Cas14 and Cm5. In one specific embodiment, a collection of nucleic acids encoding for gRNAs as provided herein comprises members that exhibit specificity for a Cpf1 protein and a Cas9 protein. In some embodiments, the nucleic acid-guided nuclease system protein-binding sequences specific for the first and second nucleic acid-guided nuclease system proteins are both 5′ of the second NA segment comprising a targeting sequence. In some embodiments, the nucleic acid-guided nuclease system protein-binding sequences specific for the first and second nucleic acid-guided nuclease system proteins are both 3′ of the second NA segment comprising a targeting sequence. In some embodiments, the nucleic acid-guided nuclease system protein-binding sequence specific for the first nucleic acid-guided nuclease system (e.g., CRISPR/Cas system) protein is 5′ of the second NA segment comprising a targeting sequence and the second nucleic acid-guided nuclease system protein-binding sequences specific for the second nucleic acid-guided nuclease system protein is 3′ of the second NA segment comprising a targeting sequence. The order of the second NA segment comprising a targeting sequence and the first NA segment comprising a nucleic acid-guided nuclease system protein-binding sequence will depend on the nucleic acid-guided nuclease system protein. The appropriate 5′ to 3′ arrangement of the first and second NA segments and choice of nucleic acid-guided nuclease system proteins will be apparent to one of ordinary skill in the art.

### Sequences of Interest

Provided herein are methods of libraries from nucleic acid samples comprising a sequence of interest, methods of enriching libraries for a sequence of interest, and methods of making collection of gNAs which can be used to enrich libraries for a sequence of interest through depletion of targeted sequences.

In some embodiments, the sequences of interest are genomic sequences (genomic DNA). In some embodiments, the sequences of interest are mammalian genomic sequences. In some embodiments, the sequences of interest are eukaryotic genomic sequences. In some embodiments, the sequences of interest are prokaryotic genomic sequences. In some embodiments, the sequences of interest are viral genomic sequences. In some embodiments, the sequences of interest are bacterial genomic sequences. In some embodiments, the sequences of interest are plant genomic sequences. In some embodiments, the sequences of interest are microbial genomic sequences. In some embodiments, the sequences of interest are genomic sequences from a parasite, for example a eukaryotic parasite. In some embodiments, the sequences of interest are host genomic sequences (e.g., the host organism of a microbiome, a parasite, or a pathogen). In some embodiments, the sequences of interest are abundant genomic sequences, such as sequences from the genome or genomes of the most abundant species in a sample.

In some embodiments, the sequences of interest comprise repetitive DNA. In some embodiments, the sequences of interest comprise abundant DNA. In some embodiments, the sequences of interest comprise mitochondrial DNA. In some embodiments, the sequences of interest comprise ribosomal DNA. In some embodiments, the sequences of interest comprise centromeric DNA. In some embodiments, the sequences of interest comprise DNA comprising Alu elements (Alu DNA). In some embodiments, the sequences of interest comprise long interspersed nuclear elements (LINE DNA). In some embodiments, the sequences of interest comprise short interspersed nuclear elements (SINE DNA). In some embodiments, the abundant DNA comprises ribosomal DNA.

In some embodiments, the sequences of interest comprise single nucleotide polymorphisms (SNPs), short tandem repeats (STRs), cancer genes, inserts, deletions, structural variations, exons, genetic mutations, or regulatory regions.

In some embodiments, the sequences of interest can be a genomic fragment, comprising a region of the genome, or the whole genome itself. In one embodiment, the genome is a DNA genome. In another embodiment, the genome is an RNA genome.

In some embodiments, the sequences of interest are from a eukaryotic or prokaryotic organism; from a mammalian organism or a non-mammalian organism; from an animal or a plant; from a bacteria or virus; from an animal parasite; from a pathogen.

In some embodiments, the sequences of interest are from any mammalian organism. In one embodiment the mammal is a human. In another embodiment the mammal is a livestock animal, for example a horse, a sheep, a cow, a pig, or a donkey. In another embodiment, a mammalian organism is a domestic pet, for example a cat, a dog, a gerbil, a mouse, a rat. In another embodiment the mammal is a type of a monkey.

In some embodiments, the sequences of interest are from any bird or avian organism. An avian organism includes but is not limited to chicken, turkey, duck and goose.

In some embodiments, the sequences of interest are from an insect. Insects include, but are not limited to honeybees, solitary bees, ants, flies, wasps or mosquitoes.

In some embodiments, the sequences of interest are from a plant. In one embodiment, the plant is rice, maize, wheat, rose, grape, coffee, fruit, tomato, potato, or cotton.

In some embodiments, the sequences of interest are from a species of bacteria. In one embodiment, the bacteria are tuberculosis-causing bacteria.

In some embodiments, the sequences of interest are from a virus.

In some embodiments, the sequences of interest are from a species of fungi.

In some embodiments, the sequences of interest are from a species of algae.

In some embodiments, the sequences of interest are from any mammalian parasite.

In some embodiments, the sequences of interest are obtained from any mammalian parasite. In one embodiment, the parasite is a worm. In another embodiment, the parasite is a malaria-causing parasite. In another embodiment, the parasite is a Leishmaniosis-causing parasite. In another embodiment, the parasite is an amoeba.

In some embodiments, the sequences of interest are from a pathogen.

In some embodiments, the sequences of interest are human sequences. In some embodiments, the human sequences are polymorphic sequences that can be used to identify individual subjects in a human population, for example single nucleotide polymorphisms (SNPs), miniSTRs (mini short tandem repeats), mitochondrial markers, Y chromosome markers, or taxonomic markers and the like.

In some embodiments, the sequence of interest comprises a disease trait marker.

In some embodiments, the sequences of interest comprise single nucleotide polymorphisms (SNPs). In some embodiments, the SNPs are used for forensic analysis of human samples. For example, the SNPs are used characterize genetic variation between subjects.

In some embodiments, the sequence of interest comprises a miniSTR. In some embodiments, the miniSTR is used for forensic analysis of human samples. For example, the miniSTR is used to characterize genetic variation between subjects.

In some embodiments, the sequences of interest comprise RNA. In some embodiments, the sequences of interest comprise a transcriptome. In some embodiments, the sequences of interest comprise sequences of specific RNA transcripts.

### Targeting Sequences

Provided herein are gNAs and collections of gNAs, derived from any source DNA (for example from genomic DNA, cDNA, artificial DNA, DNA libraries), that can be used to target sequences in a sample for a variety of applications including, but not limited to, enrichment, depletion, capture, partitioning, labeling, regulation, and editing. The gRNAs comprise a targeting sequence, directed at targeted sequences. In some embodiments, the targeted sequence comprises the sequence of interest. For example, in those embodiments where nucleic acids in a sample are partitioned using a catalytically dead CRISPR/Cas system protein. In some embodiments, the target sequence comprises a sequence of interest. In some embodiments, the targeted sequence does not comprise the sequence of interest.

Methods of the disclosure which remove untemplated 3′ nucleotides from in vitro transcription products increase the sequence identity between the targeting sequence of the gNA and the sequence of interest in the sample.

As used herein, a targeting sequence is one that directs the gNA, and therefore the gNA: CRISPR/Cas protein complex, to specific sequences in a sample. In some embodiments, a targeting sequence targets a particular sequence of interest, for example the targeting sequence targets a genomic sequence of interest. In some embodiments, the targeting sequence targets a sequence for depletion, i.e. a sequence that is not the sequence of interest. In some embodiments, the targeting sequences target sequences for depletion, thereby enriching the sample for sequences of interest.

In some embodiments, the targeting sequence does not comprise additional 3′ untemplated nucleotides. In certain embodiments, additional untemplated nucleotides introduced by in vitro transcription of a corresponding template DNA using a T7, SP6 or T3 polymerase are removed using the methods of the disclosure. In certain embodiments, the 3′ ends of the targeting sequence of a gRNA are homogenous, and these homogenous 3′ ends are identical or nearly identical to a target sequence in a sequence of interest. In certain embodiments, the homogenous 3′ ends of the targeting sequence produced by the methods of the disclosure provide superior targeting to target sites in a sequence of interest, such as a genomic DNA sequence, by reducing off-target localization of the gRNA-CRISPR/Cas protein complex. In certain embodiments, the 3′ ends of the targeting sequence of a collection of gRNAs are identical or nearly identical to the 3′ ends of their corresponding DNA templates, and this correspondence between the 3′ ends of the gRNAs and the DNA templates provides superior targeting to target sites in a sequence of interest, such as a genomic DNA sequence, by reducing off-target localization of the gRNA-CRISPR/Cas protein complex.

Provided herein are gRNAs and collections of gRNAs that comprise a segment that comprises a targeting sequence. Also provided herein, are nucleic acids encoding for gRNAs, and collections of nucleic acids encoding for gRNAs that comprise a segment encoding for a targeting sequence.

In some embodiments, the targeting sequence comprises DNA.

In some embodiments, the targeting sequence comprises RNA.

In some embodiments, the targeting sequence comprises RNA, and shares at least 70% sequence identity, at least 75% sequence identity, at least 80% sequence identity, at least 85% sequence identity, at least 90% sequence identity, at least 95% sequence identity, or shares 100% sequence identity to a sequence 3′ to a PAM sequence on a sequence of interest, except that the RNA comprises uracils instead of thymines. In some embodiments, the PAM sequence is TTN, TCN or TGN. In some embodiments, the PAM sequence is NGG or NAG.

In some embodiments, the targeting sequence comprises DNA, and shares at least 70% sequence identity, at least 75% sequence identity, at least 80% sequence identity, at least 85% sequence identity, at least 90% sequence identity, at least 95% sequence identity, or shares 100% sequence identity to a sequence 3′ to a PAM sequence on a sequence of interest. In some embodiments, the PAM sequence is TTN, TCN or TGN

In some embodiments, the targeting sequence comprises RNA and is complementary to the strand opposite to a sequence of nucleotides 3′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 3′ to a PAM sequence. In some embodiments, the PAM sequence is TTN, TCN or TGN.

In some embodiments, the targeting sequence comprises DNA and is complementary to the strand opposite to a sequence of nucleotides 3′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 3′ to a PAM sequence. In some embodiments, the PAM sequence is TTN, TCN or TGN.

In some embodiments, a DNA encoding for a targeting sequence of a gRNA shares at least 70% sequence identity, at least 75% sequence identity, at least 80% sequence identity, at least 85% sequence identity, at least 90% sequence identity, at least 95% sequence identity, or shares 100% sequence identity to the strand opposite to a sequence of nucleotides 3′ to a PAM sequence. In some embodiments, the PAM sequence is TTN, TCN or TGN.

In some embodiments, a DNA encoding for a targeting sequence of a gRNA is complementary to the strand opposite to a sequence of nucleotides 5′ to a PAM sequence and is at least 70% complementary, at least 75% complementary, at least 80% complementary, at least 85% complementary, at least 90% complementary, at least 95% complementary, or is 100% complementary to a sequence 3′ to a PAM sequence on a sequence of interest. In some embodiments, the PAM sequence is TTN, TCN or TGN.

In some embodiments, the targeting sequence comprises RNA, and shares at least 70% sequence identity, at least 75% sequence identity, at least 80% sequence identity, at least 85% sequence identity, at least 90% sequence identity, at least 95% sequence identity, or shares 100% sequence identity to a sequence 5′ to a PAM sequence on a sequence of interest, except that the RNA comprises uracils instead of thymines. In some embodiments, the PAM sequence is NGG or NAG.

In some embodiments, the targeting sequence comprises DNA, and shares at least 70% sequence identity, at least 75% sequence identity, at least 80% sequence identity, at least 85% sequence identity, at least 90% sequence identity, at least 95% sequence identity, or shares 100% sequence identity to a sequence 5′ to a PAM sequence on a sequence of interest. In some embodiments, the PAM sequence is NGG or NAG.

In some embodiments, the targeting sequence comprises RNA and is complementary to the strand opposite to a sequence of nucleotides 5′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 5′ to a PAM sequence. In some embodiments, the PAM sequence is NGG or NAG.

In some embodiments, the targeting sequence comprises DNA and is complementary to the strand opposite to a sequence of nucleotides 5′ to a PAM sequence. In some embodiments, the targeting sequence is at least 70% complementary, at least 75% complementary, at least 80% complementary, at least 85% complementary, at least 90% complementary, at least 95% complementary, or is 100% complementary to the strand opposite to a sequence of nucleotides 5′ to a PAM sequence. In some embodiments, the PAM sequence is NGG or NAG.

In some embodiments, a DNA encoding for a targeting sequence of a gRNA shares at least 70% sequence identity, at least 75% sequence identity, at least 80% sequence identity, at least 85% sequence identity, at least 90% sequence identity, at least 95% sequence identity, or shares 100% sequence identity to the strand opposite to a sequence of nucleotides 5′ to a PAM sequence. In some embodiments, the PAM sequence is NGG or NAG.

In some embodiments, a DNA encoding for a targeting sequence of a gRNA is complementary to the strand opposite to a sequence of nucleotides 5′ to a PAM sequence and is at least 70% complementary, at least 75% complementary, at least 80% complementary, at least 85% complementary, at least 90% complementary, at least 95% complementary, or is 100% complementary to a sequence 5′ to a PAM sequence on a sequence of interest. In some embodiments, the PAM sequence is NGG or NAG.

### Nucleic Acid-Guided Nuclease System Proteins

Provided herein are gNAs and collections of gNAs comprising a segment that comprises a nucleic acid-guided nuclease system (e.g., CRISPR/Cas system) protein-binding sequence (e.g., a stem loop sequence). Also provided herein, are nucleic acids encoding for gNAs (e.g. gRNAs), and collections of nucleic acids encoding for gRNAs that comprise a segment encoding a nucleic acid-guided nuclease system (e.g., CRISPR/Cas system) protein-binding sequence. A nucleic acid-guided nuclease system can be an RNA-guided nuclease system.

Methods of the present disclosure can utilize nucleic acid-guided nucleases. As used herein, a “nucleic acid-guided nuclease” is any nuclease that cleaves DNA, RNA or DNA/RNA hybrids, and which uses one or more nucleic acid guide nucleic acids (gRNAs) to confer specificity. Nucleic acid-guided nucleases include CRISPR/Cas system proteins as well as non-CRISPR/Cas system proteins.

The nucleic acid-guided nucleases provided herein can be RNA guided DNA nucleases or RNA guided RNA nucleases. The nucleases can be endonucleases. The nucleases can be exonucleases. In one embodiment, the nucleic acid-guided nuclease is a nucleic acid-guided-DNA endonuclease. In one embodiment, the nucleic acid-guided nuclease is a nucleic acid-guided-RNA endonuclease.

A nucleic acid-guided nuclease system protein-binding sequence is a nucleic acid sequence that binds any protein member of a nucleic acid-guided nuclease system. For example, a CRISPR/Cas system protein-binding sequence is a nucleic acid sequence that binds any protein member of a CRISPR/Cas system.

Provided herein are gRNAs and collections of gRNAs which comprises a 5′ segment encoding a nucleic acid-guided nuclease system protein-binding sequence and a 3′ segment encoding targeting sequence through in vitro transcription. All CRISPR/Cas system proteins compatible with this 5′ to 3′ arrangement of segments in the gRNA are within the scope of the invention.

Exemplary nucleic acid-guided nucleases are selected from the group consisting of CAS Class I Type I, CAS Class I Type III, CAS Class I Type IV, CAS Class II Type II, and CAS Class II Type V. In some embodiments, CRISPR/Cas system proteins include proteins from CRISPR Type I systems, CRISPR Type II systems, and CRISPR Type III systems. Exemplary nucleic acid-guided nucleases include, but are not limited to, Cas9, Cpf1, Cas10, Csm2, CasX, CasY and C2c2.

In some embodiments, nucleic acid-guided nuclease system proteins (e.g., CRISPR/Cas system proteins) can be from any bacterial or archaeal species.

In some embodiments, the nucleic acid-guided nuclease system proteins (e.g., CRISPR/Cas system proteins) are from, or are derived from nucleic acid-guided nuclease system proteins (e.g., CRISPR/Cas system proteins) from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, Corynebacter diphtheria, Acidaminococcus, Lachnospiraceae bacterium or Prevotella.

In some embodiments, examples of nucleic acid-guided nuclease system (e.g., CRISPR/Cas system) proteins can be naturally occurring or engineered versions.

In some embodiments, naturally occurring nucleic acid-guided nuclease system (e.g., CRISPR/Cas system) proteins Exemplary nucleic acid-guided nucleases include, but are not limited to, Cas9, Cpf1, Cas10, Csm2, CasX, CasY and C2c2. Engineered versions of such proteins can also be employed.

In some embodiments, engineered examples of nucleic acid-guided nuclease system (e.g., CRISPR/Cas system) proteins include catalytically dead nucleic acid-guided nuclease system proteins. The term “catalytically dead” generally refers to a nucleic acid-guided nuclease system protein that has inactivated nucleases (e.g., RuvC nucleases). Such a protein can bind to a target site in any nucleic acid (where the target site is determined by the guide NA), but the protein is unable to cleave or nick the target nucleic acid (e.g., double-stranded DNA). In some embodiments, the nucleic acid-guided nuclease system catalytically dead protein is a catalytically dead CRISPR/Cas system protein. Accordingly, the catalytically dead CRISPR/Cas system protein allows separation of the mixture into unbound nucleic acids and protein-bound fragments. In one embodiment, a catalytically dead CRISPR/Cas system protein complex binds to targets determined by the gRNA sequence. The catalytically dead CRISPR/Cas system protein bound can prevent cutting by the CRISPR/Cas system protein while other manipulations proceed. In another embodiment, the catalytically dead CRISPR/Cas system protein can be fused to another enzyme, such as a transposase, to target that enzyme's activity to a specific site. Naturally occurring catalytically dead nucleic acid-guided nuclease system proteins can also be employed.

In some embodiments, engineered examples of nucleic acid-guided nuclease (e.g., CRISPR/Cas) system proteins also include nucleic acid-guided nickases (e.g., Cas nickases). A nucleic acid-guided nickase refers to a modified version of a nucleic acid-guided nuclease system protein, containing a single inactive catalytic domain. In one embodiment, the nucleic acid-guided nickase is a Cas nickase, for example a Cas9 nickase. A Cas nickase may contain a single inactive catalytic domain, for example, the RuvC domain. With only one active nuclease domain, the Cas nickase cuts only one strand of the target DNA, creating a single-strand break or “nick”. Depending on which mutant is used, the guide NA-hybridized strand or the non-hybridized strand may be cleaved. Nucleic acid-guided nickases bound to 2 gRNAs that target opposite strands will create a double-strand break in a target double-stranded DNA. This “dual nickase” strategy can increase the specificity of cutting because it requires that both nucleic acid-guided nuclease/gRNA complexes be specifically bound at a site before a double-strand break is formed. Naturally occurring nickase nucleic acid-guided nuclease system proteins can also be employed.

In some embodiments, engineered examples of nucleic acid-guided nuclease system proteins also include nucleic acid-guided nuclease system fusion proteins. For example, a nucleic acid-guided nuclease (e.g., CRISPR/Cas) system protein may be fused to another protein, for example an activator, a repressor, a nuclease, a fluorescent molecule, a radioactive tag, or a transposase.

In some embodiments, the nucleic acid-guided nuclease system protein-binding sequence comprises a gRNA stem-loop sequence.

Different CRISPR/Cas system proteins are compatible with different nucleic acid-guided nuclease system protein-binding sequences. It will be readily apparent to one of ordinary skill in the art which CRISPR/Cas system proteins are compatible with which nucleic acid-guided nuclease system protein-binding sequences.

In some embodiments, the CRISPR/Cas system protein is a Cpf1 protein. In some embodiments, the Cpf1 protein is isolated or derived from Franciscella species or Acidaminococcus species. In some embodiments, the gRNA CRISPR/Cas system protein-binding sequence comprises the following RNA sequence: (5′>3′, AAUUUCUACUGUUGUAGAU) (SEQ ID NO: 7).

In some embodiments, the CRISPR/Cas system protein is a Cpf1 protein. In some embodiments, the Cpf1 protein is isolated or derived from Franciscella species or Acidaminococcus species. In some embodiments, a DNA sequence encoding the gRNA CRISPR/Cas system protein-binding sequence comprises the following DNA sequence: (5′>3′, AATTTCTACTGTTGTAGAT) (SEQ ID NO: 8). In some embodiments, the DNA is single stranded. In some embodiments, the DNA is double stranded.

In some embodiments, provided herein is a nucleic acid encoding for a gRNA comprising a first segment comprising a regulatory region; a second segment comprising a nucleic acid encoding a nucleic acid-guided nuclease (e.g., CRISPR/Cas) system protein-binding sequence; and a third segment encoding a targeting sequence. In some embodiments, for example those embodiments wherein the CRISPR/Cas system protein is a Cpf1 system protein, the first, second and third segments are arranged, from 5′ to 3′: first segment (regulatory region), second segment (nucleic acid-guided nuclease system protein-binding sequence), and third segment (targeting sequence). In some embodiments, the second segment comprises a single transcribed component, which upon transcription yields a NA (e.g., RNA) stem-loop sequence. In some embodiments, the second segment comprising a single transcribed component that encodes for the gRNA stem-loop sequence is double-stranded, comprises the following DNA sequence on one strand (5′>3′, AATTTCTACTGTTGTAGAT) (SEQ ID NO: 8), and its reverse-complementary DNA on the other strand (5′>3′, ATCTACAACAGTAGAAATT) (SEQ ID NO: 9). In some embodiments, the second segment comprising a single transcribed component that encodes for the gRNA stem-loop sequence is single-stranded, and comprises the following DNA sequence: (5′>3′, ATCTACAACAGTAGAAATT) (SEQ ID NO: 9), wherein the single-stranded DNA serves as a transcription template. In some embodiments, upon transcription from the single transcribed component, the resulting gRNA stem-loop sequence comprises the following RNA sequence: (5′>3′, AAUUUCUACUGUUGUAGAU) (SEQ ID NO: 7).

In some embodiments, provided herein is a nucleic acid encoding for a gRNA comprising a first segment comprising a regulatory region; a second segment comprising a nucleic acid encoding a nucleic acid-guided nuclease (e.g., CRISPR/Cas) system protein-binding sequence; and a third segment encoding a targeting sequence. In some embodiments, for example those embodiments wherein the CRISPR/Cas system protein is a Cpf1 system protein, the first, second and third segments are arranged, from 5′ to 3′: first segment (regulatory region), second segment (nucleic acid-guided nuclease system protein-binding sequence), and third segment (targeting sequence). In some embodiments, the second segment comprises a single transcribed component, which upon transcription yields an RNA stem-loop sequence. In some embodiments, the second segment comprising a single transcribed component that encodes for the gRNA stem-loop sequence is double-stranded, comprises the following DNA sequence on one strand (5′>3′, AATTTCTACTGTTGTAGAT) (SEQ ID NO: 8), and its reverse-complementary DNA on the other strand (5′>3′, ATCTACAACAGTAGAAATT) (SEQ ID NO: 9). In some embodiments, the second segment comprising a single transcribed component that encodes for the gRNA stem-loop sequence is single-stranded, and comprises the following DNA sequence: (5′>3′, ATCTACAACAGTAGAAATT) (SEQ ID NO: 9), wherein the single-stranded DNA serves as a transcription template. In some embodiments, upon transcription from the single transcribed component, the resulting gRNA stem-loop sequence comprises the following RNA sequence: (5′>3′, AAUUUCUACUGUUGUAGAU) (SEQ ID NO: 7).

In some embodiments, provided herein is a nucleic acid encoding for a gRNA comprising a first segment comprising a regulatory region; a second segment comprising a nucleic acid encoding a nucleic acid-guided nuclease (e.g., CRISPR/Cas) system protein-binding sequence; and a third segment encoding a targeting sequence. In some embodiments, for example those embodiments wherein the CRISPR/Cas system protein is a Cas9 system protein, the first, second and third segments are arranged, from 5′ to 3′: first segment (regulatory region), third segment (targeting sequence), and second segment (nucleic acid-guided nuclease system protein-binding sequence). In some embodiments, the second segment (nucleic acid-guided nuclease system protein-binding sequence) comprises a stem-loop sequence. In some embodiments, a double-stranded DNA sequence encoding the gNA (e.g., gRNA) stem-loop sequence comprises the following DNA sequence on one strand (5′>3′, GTTTTAGAGCTAGAAATAGCAAGTTAAAATAAGGCTAGTCCGTTATCAACTTGAAAAA GTGGCACCGAGTCGGTGCTTTTTTT) (SEQ ID NO: 10), and its reverse-complementary DNA on the other strand (5′>3′, AAAAAAAGCACCGACTCGGTGCCACTTTTTCAAGTTGATAACGGACTAGCCTTATTTTA ACTTGCTATTTCTAGCTCTAAAAC) (SEQ ID NO: 11). In some embodiments, a single-stranded DNA sequence encoding the gNA (e.g., gRNA) stem-loop sequence comprises the following DNA sequence: (5′>3′, AAAAAAAGCACCGACTCGGTGCCACTTTTTCAAGTTGATAACGGACTAGCCTTATTTTA ACTTGCTATTTCTAGCTCTAAAAC) (SEQ ID NO: 11), wherein the single-stranded DNA serves as a transcription template. In some embodiments, the gNA (e.g., gRNA) stem-loop sequence comprises the following RNA sequence: (5′>3′, GUUUUAGAGCUAGAAAUAGCAAGUUAAAAUAAGGCUAGUCCGUUAUCAACUUGAAA AAGUGGCACCGAGUCGGUGCUUUUUUU) (SEQ ID NO: 12).

In some embodiments, the regulatory sequence can be bound by a transcription factor. In some embodiments, the regulatory sequence is a promoter. In some embodiments, the regulatory sequence is a T7 promoter, comprising a sequence of 5′-GCCTCGAGCTAATACGACTCACTATAGAG-3′ (SEQ ID NO: 3). In some embodiments, the T7 promoter comprises a sequence of 5′-TAATACGACTCACTATAGG-3′ (SEQ ID NO: 1). In some embodiments, the T7 promoter comprises a sequence of 5′-TAATACGACTCACTATAGGG-3′ (SEQ ID NO: 2). In some embodiments, the regulatory sequence is an SP6 promoter. In some embodiments, the SP6 promoter comprises a sequence of 5′-ATTTAGGTGACACTATAG-3′ (SEQ ID NO: 4). In some embodiments, the SP6 promoter comprises a sequence of 5′-CATACGATTTAGGTGACACTATAG-3′ (SEQ ID NO: 5). In some embodiments, the regulatory sequence is a T3 promoter. In some embodiments, the T3 promoter comprises a sequence of 5′ AATTAACCCTCACTAAAG 3′ (SEQ ID NO: 6).

### CRISPR Cas System Nucleic Acid-Guided Nucleases

In some embodiments, CRISPR/Cas system proteins are used in the embodiments provided herein. In some embodiments, CRISPR/Cas system proteins include proteins from CRISPR Type I systems, CRISPR Type II systems, and CRISPR Type III systems.

In some embodiments, CRISPR/Cas system proteins can be from any bacterial or archaeal species.

In some embodiments, the CRISPR/Cas system protein is isolated, recombinantly produced, or synthetic.

In some embodiments, the CRISPR/Cas system proteins are from, or are derived from CRISPR/Cas system proteins from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, Corynebacter diphtheria, Acidaminococcus, Lachnospiraceae bacterium or Prevotella.

In some embodiments, examples of CRISPR/Cas system proteins can be naturally occurring or engineered versions.

In some embodiments, naturally occurring CRISPR/Cas system proteins can belong to CAS Class I Type I, III, or IV, or CAS Class II Type II or V, and can include Cpf1, Cas10, Csm2 and C2c2.

In some embodiments, CRISPR/Cas system proteins can belong to CAS Class I Type I, III, or IV, or CAS Class II Type II or V, and can include Cas9, Cas3, Cas8a-c, Cas10, CasX, CasY, Cas13, Cas14, Cse1, Csy1, Csn2, Cas4, Csm2, Cmr5, Csf1, C2c2, and Cpf1.

In an exemplary embodiment, the CRISPR/Cas system protein comprises Cpf1.

In an exemplary embodiment, the CRISPR/Cas system protein comprises Cas9.

A “CRISPR/Cas system protein-gRNA complex” refers to a complex comprising a CRISPR/Cas system protein and a guide NA (e.g. a gRNA or a gDNA). The gRNA may be a single molecule (i.e. a gRNA) that comprises a crRNA sequence.

A CRISPR/Cas system protein may be at least 60% identical (e.g., at least 70%, at least 80%, or 90% identical, at least 95% identical or at least 98% identical or at least 99% identical) to a wild type CRISPR/Cas system protein. The CRISPR/Cas system protein may have all the functions of a wild type CRISPR/Cas system protein, or only one or some of the functions, including binding activity, nuclease activity, and nuclease activity.

The term “CRISPR/Cas system protein-associated guide RNA” refers to a guide RNA. The CRISPR/Cas system protein-associated guide RNA may exist as isolated RNA, or as part of a CRISPR/Cas system protein-gRNA complex.

All CRISPR/Cas system proteins compatible with gRNAs with a 5′ nucleic acid-guided nuclease system protein binding sequence and a 3′ targeting sequence are within the scope of the invention.

In some embodiments, the CRISPR/Cas system protein is an RNA-guided RNA nuclease (i.e., cuts RNA). Exemplary CRISPR/Cas system proteins that cut RNA include, but are not limited to C2c2. C2c2 (also known as Cas13a) is a class 2 type VI RNA-guided RNA-targeting CRISPR/Cas system protein. In some embodiments, the C2c2 nuclease is isolated or derived from Leptotrichia shahii. In some embodiments, C2c2 is guided by a single crRNA that cleaves an ssRNA carrying a complementary protospacer. An appropriate C2c2 crRNA sequence will be readily apparent to one of ordinary skill in the art.

In some embodiments, the CRISPR/Cas system protein is an RNA-guided DNA nuclease. In some embodiments, the DNA cleaved by the CRISPR/Cas system protein is double stranded. Exemplary RNA-guided DNA nucleases that cut double stranded DNA include, but are not limited to Cas9, Cpf1, CasX and CasY. Further exemplary RNA-guided DNA nucleases include Cas10, Csm2, Csm3, Csm4, and Csm5. In some embodiments, Cas10, Csm2, Csm3, Csm4, and Csm5 form a ribonucleoprotein complex with a gRNA.

In some embodiments, the RNA-guided DNA nuclease is CasX. In some embodiments, the CasX protein is dual guided (i.e., the gNA comprises a crRNA and a tracrRNA). In some embodiments, CasX recognizes a TTCN PAM located immediately 5′ of a sequence complementary to the targeting sequence. In some embodiments, the CasX protein is isolated or derived from Deltaproteobacteria or Planctomycetes. In some embodiments, the CasX protein is a CasX1, a CasX2 or a CasX3 protein. CasX proteins are described in WO/2018/064371, the contents of which are incorporated herein by reference in their entirety. Appropriate gNA sequences for CasX proteins will be readily apparent to the person of ordinary skill in the art.

In some embodiments, the RNA-guided DNA nuclease is CasY. In some embodiments, the CasY protein is dual guided (i.e., the gNA comprises a crRNA and a tracrRNA). In some embodiments, CasY recognizes a TA PAM located 5′ of the target sequence. CasY proteins are described in WO/2018/064352, the contents of which are incorporated herein by reference in their entirety. Appropriate gNA sequences for CasY proteins will be readily apparent to the person of ordinary skill in the art.

In some embodiments, the CRISPR/Cas system protein is an RNA-guided DNA nuclease. In some embodiments, the DNA cleaved by the CRISPR/Cas system protein is single stranded. Exemplary RNA guided CRISPR/Cas system proteins that cut single stranded DNA include, but are not limited to, Cas3 and Cas14. In some embodiments, the Cas14 protein does not require a PAM site.

### Cas9

In some embodiments, the CRISPR/Cas System protein nucleic acid-guided nuclease is or comprises Cas9. The Cas9 of the present disclosure can be isolated, recombinantly produced, or synthetic.

Examples of Cas9 proteins that can be used in the embodiments herein can be found in F. A. Ran, L. Cong, W. X. Yan, D. A. Scott, J. S. Gootenberg, A. J. Kriz, B. Zetsche, O. Shalem, X. Wu, K. S. Makarova, E. V. Koonin, P. A. Sharp, and F. Zhang; “In vivo genome editing using Staphylococcus aureus Cas9,” Nature 520, 186-191 (9 Apr. 2015) doi: 10.1038/nature14299, which is incorporated herein by reference.

In some embodiments, the Cas9 is a Type II CRISPR system derived from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

In some embodiments, the Cas9 is a Type II CRISPR system derived from S. pyogenes and the PAM sequence is NGG or NAG located on the immediate 3′ end of the target specific guide sequence. The PAM sequences of Type II CRISPR systems from exemplary bacterial species can also include: Streptococcus pyogenes (NGG), Staphylococcus aureus (NNGRRT), Neisseria meningitidis (NNNNGATT), Streptococcus thermophilus (NNAGAA) and Treponema denticola (NAAAAC) which are all usable without deviating from the present disclosure.

In one exemplary embodiment, Cas9 sequence can be obtained, for example, from the pX330 plasmid (available from Addgene), re-amplified by PCR then cloned into pET30 (from EMD biosciences) to express in bacteria and purify the recombinant 6His tagged protein.

A “Cas9-gNA complex” refers to a complex comprising a Cas9 protein and a guide NA. A Cas9 protein may be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein may have all the functions of a wild type Cas9 protein, or only one or some of the functions, including binding activity, nuclease activity, and nuclease activity.

The term “Cas9-associated guide NA” refers to a guide NA as described above. The Cas9-associated guide NA may exist isolated, or as part of a Cas9-gNA complex.

### Non-CRISPR Cas System Proteins

In some embodiments, non-CRISPR/Cas system proteins are used in the embodiments provided herein.

In some embodiments, the non-CRISPR/Cas system proteins can be from any bacterial or archaeal species.

In some embodiments, the non-CRISPR/Cas system protein is isolated, recombinantly produced, or synthetic.

In some embodiments, the non-CRISPR/Cas system proteins are from, or are derived from Aquifex aeolicus, Thermus thermophilus, Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, Natronobacterium gregoryi, or Corynebacter diphtheria.

In some embodiments, the non-CRISPR/Cas system proteins can be naturally occurring or engineered versions.

In some embodiments, a naturally occurring non-CRISPR/Cas system protein is NgAgo (Argonaute from Natronobacterium gregoryi).

A “non-CRISPR/Cas system protein-gNA complex” refers to a complex comprising a non-CRISPR/Cas system protein and a guide NA (e.g. a gRNA or a gDNA). Where the gNA is a gRNA, the gRNA may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a gRNA) that contains crRNA and tracrRNA sequences.

A non-CRISPR/Cas system protein may be at least 60% identical (e.g., at least 70%, at least 80%, or 90% identical, at least 95% identical or at least 98% identical or at least 99% identical) to a wild type non-CRISPR/Cas system protein. The non-CRISPR/Cas system protein may have all the functions of a wild type non-CRISPR/Cas system protein, or only one or some of the functions, including binding activity, nuclease activity, and nuclease activity.

The term “non-CRISPR/Cas system protein-associated guide NA” refers to a guide NA. The non-CRISPR/Cas system protein-associated guide NA may exist as isolated NA, or as part of a non-CRISPR/Cas system protein-gNA complex.

### Cpf1

In some embodiments, the CRISPR/Cas system protein nucleic acid-guided nuclease is or comprises a Cpf1 system protein. Cpf1 system proteins of the present invention can be isolated, recombinantly produced, or synthetic.

Cpf1 system proteins are Class II, Type V CRISPR system proteins. In some embodiments, the Cpf1 protein is isolated or derived from Francisella tularensis. In some embodiments, the Cpf1 protein is isolated or derived from Acidaminococcus, Lachnospiraceae bacterium or Prevotella.

Cpf1 proteins bind to a single guide RNA comprising a nucleic acid-guided nuclease system protein-binding sequence (e.g., stem-loop) and a targeting sequence. The Cpf1 targeting sequence comprises a sequence located immediately 3′ of a Cpf1 PAM sequence in a target nucleic acid. Unlike Cas9, the Cpf1 nucleic acid-guided nuclease system protein-binding sequence is located 5′ of the targeting sequence in the Cpf1 gRNA. Cpf1 can also produce staggered rather than blunt ended cuts in a target nucleic acid. Following targeting of the Cpf1 protein-gRNA complex to a target nucleic acid, Francisella derived Cpf1, for example, cleaves the target nucleic acid in a staggered fashion, creating an approximately 5 nucleotide 5′ overhang 18-23 bases away from the PAM at the 3′ end of the targeting sequence. In contrast, cutting by a wild type Cas9 produces a blunt end 3 nucleotides upstream of the Cas9 PAM.

In some embodiments, the CRISPR/Cas system protein is a Cpf1 system protein. Cpf1 system proteins can be isolated or derived from a variety of bacteria species, including, but not limited to, Francisella tularensis, Acidaminococcus, Lachnospiraceae bacterium or Prevotella. Cpf1 system proteins isolated or derived from different species can recognize and bind to different nucleic acid-guided nuclease system protein-binding sequences (sometimes called stem loop sequences). An exemplary Cpf1 system protein nucleic acid-guided nuclease system protein-binding sequence comprises the following RNA sequence: (5′>3′, AAUUUCUACUGUUGUAGAU) (SEQ ID NO: 7). A person of ordinary skill in the art will understand how to select nucleic acid-guided nuclease system protein-binding sequences that bind Cpf1 system proteins.

A “Cpf1 protein-gRNA complex” refers to a complex comprising a Cpf1 protein and a guide NA (e.g. a gRNA or a gDNA). The gRNA may be composed of a single molecule, i.e., one RNA (“crRNA”) which hybridizes to a target and provides sequence specificity.

A Cpf1 protein may be at least 60% identical (e.g., at least 70%, at least 80%, or 90% identical, at least 95% identical or at least 98% identical or at least 99% identical) to a wild type Cpf1 protein. The Cpf1 protein may have all the functions of a wild type Cpf1 protein, or only one or some of the functions, including binding activity, and nuclease activity.

Cpf1 system proteins recognize a variety of PAM sequences. Exemplary PAM sequences recognized by Cpf1 system proteins include, but are not limited to TTN, TCN and TGN. Additional Cpf1 PAM sequences include, but are not limited to TTTN.

One feature of Cpf1 PAM sequences is that they have a higher A/T content than the NGG or NAG PAM sequences used by Cas9 proteins. Target nucleic acids, for example, different genomes, differ in their percent G/C content. For example, the genome of the human malaria parasite Plasmodium falciparum is known to be A/T rich. Alternatively, protein coding sequences within a genome frequently have a higher G/C content than the genome as a whole. The ratio of A/T to G/C nucleotides in a target genome affects the distribution and frequency of a given PAM sequence in that genome. For example, A/T rich genomes may have fewer NGG or NAG sequences, while G/C rich genomes may have fewer TTN sequences. Cpf1 system proteins expand the repertoire of PAM sequences available to the ordinarily skilled artisan, resulting superior flexibility and function of gRNA libraries.

### Catalytically Dead Nucleic Acid-Guided Nucleases

In some embodiments, engineered examples of nucleic acid-guided nucleases include catalytically dead nucleic acid-guided nucleases (CRISPR/Cas system nucleic acid-guided nucleases or non-CRISPR/Cas system nucleic acid-guided nucleases). The term “catalytically dead” generally refers to a nucleic acid-guided nuclease that has inactivated nucleases, for example inactivated RuvC nucleases. Such a protein can bind to a target site in any nucleic acid (where the target site is determined by the guide NA), but the protein is unable to cleave or nick the nucleic acid.

In some embodiments, the catalytically dead nucleic acid-guided nuclease can be fused to another enzyme, such as a transposase, to target that enzyme's activity to a specific site.

In exemplary embodiments, the catalytically dead nucleic acid-guided nuclease protein is a dCpf1 protein.

In exemplary embodiments, the catalytically dead nucleic acid-guided nuclease protein is a dCas9 protein.

### Nucleic Acid-Guided Nuclease Nickases

In some embodiments, engineered examples of nucleic acid-guided nucleases include nucleic acid-guided nuclease nickases (referred to interchangeably as nickase nucleic acid-guided nucleases).

In some embodiments, engineered examples of nucleic acid-guided nucleases include CRISPR/Cas system nickases or non-CRISPR/Cas system nickases, containing a single inactive catalytic domain.

In exemplary embodiments, the nucleic acid-guided nuclease nickase is a Cpf1 nickase.

In exemplary embodiments, the nucleic acid-guided nuclease nickase is a Cas9 nickase.

In some embodiments, a nucleic acid-guided nuclease nickase can be used to bind to target sequence. With only one active nuclease domain, the nucleic acid-guided nuclease nickase cuts only one strand of a target DNA, creating a single-strand break or “nick”.

In exemplary embodiments, a Cas9 or Cpf1 nickase can be used to bind to target sequence. The term “Cpf1 nickase” refers to a modified version of the Cpf1 protein, containing a single inactive catalytic domain, for example, the RuvC domain. The term “Cas9 nickase” refers to a modified version of the Cas9 protein, containing a single inactive catalytic domain, for example, the RuvC domain. With only one active nuclease domain, the Cas9 or Cpf1 nickase cuts only one strand of the target DNA, creating a single-strand break or “nick”. Cas9 or Cpf1 nickases bound to 2 gRNAs that target opposite strands will create a double-strand break in the DNA. This “dual nickase” strategy can increase the specificity of cutting because it requires that both Cas9 or Cpf1/gRNA complexes be specifically bound at a site before a double-strand break is formed.

Capture of DNA can be carried out using a nucleic acid-guided nuclease nickase. In one exemplary embodiment, a nucleic acid-guided nuclease nickase cuts a single strand of double stranded nucleic acid, wherein the double stranded region comprises methylated nucleotides.

### Dissociable and Thermostable Nucleic Acid-Guided Nucleases

In some embodiments, thermostable nucleic acid-guided nucleases are used in the methods provided herein (thermostable CRISPR/Cas system nucleic acid-guided nucleases or thermostable non-CRISPR/Cas system nucleic acid-guided nucleases). In such embodiments, the reaction temperature is elevated, inducing dissociation of the protein; the reaction temperature is lowered, allowing for the generation of additional cleaved target sequences. In some embodiments, thermostable nucleic acid-guided nucleases maintain at least 50% activity, at least 55% activity, at least 60% activity, at least 65% activity, at least 70% activity, at least 75% activity, at least 80% activity, at least 85% activity, at least 90% activity, at least 95% activity, at least 96% activity, at least 97% activity, at least 98% activity, at least 99% activity, or 100% activity, when maintained for at least 75° C. for at least 1 minute. In some embodiments, thermostable nucleic acid-guided nucleases maintain at least 50% activity, when maintained for at least 1 minute at least at 75° C., at least at 80° C., at least at 85° C., at least at 90° C., at least at 91° C., at least at 92° C., at least at 93° C., at least at 94° C., at least at 95° C., 96° C., at least at 97° C., at least at 98° C., at least at 99° C., or at least at 100° C. In some embodiments, thermostable nucleic acid-guided nucleases maintain at least 50% activity, when maintained at least at 75° C. for at least 1 minute, 2 minutes, 3 minutes, 4 minutes, or 5 minutes. In some embodiments, a thermostable nucleic acid-guided nuclease maintains at least 50% activity when the temperature is elevated, lowered to 25° C.-50° C. In some embodiments, the temperature is lowered to 25° C., to 30° C., to 35° C., to 40° C., to 45° C., or to 50° C. In one exemplary embodiment, a thermostable enzyme retains at least 90% activity after 1 min at 95° C.

In some embodiments, the thermostable CRISPR/Cas system protein is thermostable Cpf1.

In some embodiments, the thermostable CRISPR/Cas system protein is thermostable Cas9.

Thermostable nucleic acid-guided nucleases can be isolated, for example, identified by sequence homology in the genome of thermophilic bacteria Streptococcus thermophilus and Pyrococcus furiosus. Nucleic acid-guided nuclease genes can then be cloned into an expression vector.

In other embodiments, a thermostable nucleic acid-guided nuclease can be obtained by in vitro evolution of a non-thermostable nucleic acid-guided nuclease. The sequence of a nucleic acid-guided nuclease can be mutagenized to improve its thermostability.

Methods of Making Collections of gRNAs

Provided herein are methods that enable the generation of a large number of diverse gRNAs, collections of gRNAs, from any source nucleic acid (e.g., DNA) that can be used with CRISPR/Cas system endonucleases. Some methods for the efficient synthesis of collections of gRNAs with a 3′ nucleic acid guided nuclease system protein binding sequence and a 5′ targeting sequence may be specific to gRNAs with that arrangement of segments. Provided herein are methods for the synthesis of collections of gRNAs with a 5′ nucleic acid guided nuclease system protein binding sequence and a 3′ targeting sequence. All CRISPR/Cas endonucleases that are compatible with gRNAs with a 5′ nucleic acid guided nuclease system protein binding sequence and a 3′ targeting sequence are envisaged as within the scope of the methods of the disclosure.

Provided herein are methods of making in vitro transcribed gRNAs from a corresponding DNA nucleic acid source using a polymerase such as T7, SP6 or T3. Polymerases such as T7, SP6 and T3 can add untemplated nucleotides at the 3′ end of a gRNA. For Cpf1 system protein compatible gRNAs, the arrangement of the nucleic acid guided nuclease system protein-binding sequence relative the targeting sequence makes these additional nucleotides potentially problematic. Provided herein are methods and compositions to remove additional 3′ nucleotides from gRNAs to generate gRNAs and collections of gRNAs with 3′ ends that do not contain additional untemplated 3′ nucleotides.

The contents of the PCT publication WO/2017/100343 and the PCT Application entitled “CREATION AND USE OF GUIDE NUCLEIC ACIDS” filed on Jun. 7, 2018, which describe compositions and methods for making collections of gRNAs, are hereby incorporated by reference in their entireties.

Methods provided herein can employ enzymatic methods including but not limited to digestion, ligation, extension, overhang filling, transcription, reverse transcription and amplification.

In some embodiments, the method comprises providing a nucleic acid (e.g., DNA); employing a first enzyme (or combinations of first enzymes) that cuts at a part of the PAM sequence in the nucleic acid, in a way that a residual nucleotide sequence from the PAM sequence is left; ligating an adapter that positions a restriction enzyme type IIS site (an enzyme that cuts outside yet near its recognition motif) at a distance to eliminate the PAM sequence; employing a second type IIS enzyme (or combination of second enzymes) to eliminate the PAM sequence together with the adapter; and fusing a sequence that can be recognized by protein members of the nucleic acid-guided nuclease (e.g., CRISPR/Cas) system, for example, a gRNA stem-loop sequence. In some embodiments, the first enzymatic reactions cuts part of the PAM sequence in a way that residual nucleotide sequence from the PAM sequence is left, and that the nucleotide sequence immediately 3′ to the PAM sequence can be any purine or pyrimidine. Alternative strategies for fragmenting a provided nucleic acid (e.g. DNA) specifically at the Cpf1 PAM sites comprise replacing adenines with inosines, or thymidines with uracils, and then cutting at abasic or mismatched sites.

As an additional alternative, a provided nucleic acid (e.g. DNA) can be randomly sheared. By random chance, a proportion of the fragmentation sites generated by random shearing will overlap with TTN PAM sequences. The fragments can be ligated either to adapters with complementary overhangs, or to blunt ended adapters that reconstitute functional restriction sites only when ligated to a fragment with a terminal PAM. These strategies allow for the selective processing into gRNAs of only those fragments that were 3′ of a PAM sequence in the original nucleic acid provided.

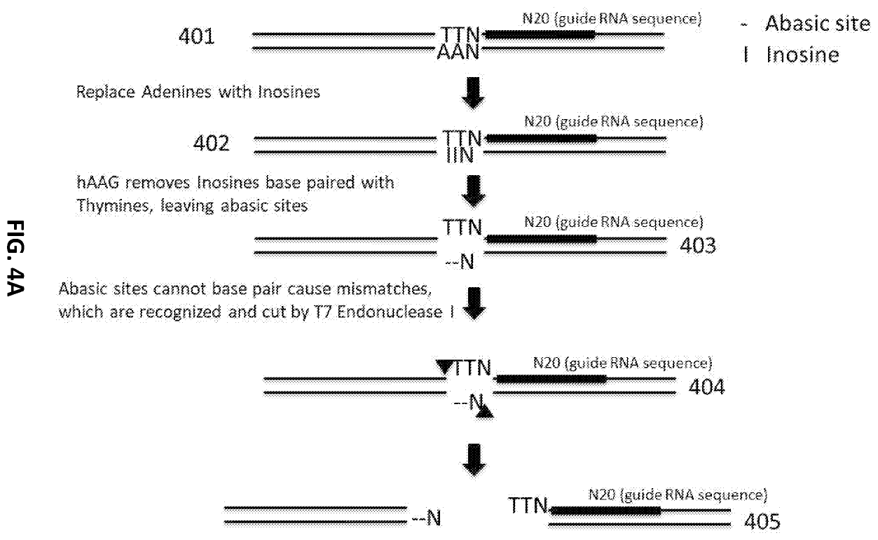

FIG. 3 shows an additional technique for constructing a gRNA library from input nucleic acids (e.g., DNA), such as genomic DNA (e.g., human genomic DNA, reverse transcribed cDNA such as from mRNA). The protocol can begin with nucleic acid fragments that have been cut with either MseI (301) or MluCI (302). MseI cuts within TTAA sites, while MluCI cuts at AATT sites. Both MseI and MluCI recognition sites comprise TTN, which, in certain embodiments, functions as a PAM site. For example, Cpf1 proteins isolated from Francisella tularensis recognize TTN as a PAM. Starting DNA digested with MseI or MluCI results in a collection of digested fragments such that the ends of the fragments comprise potential PAM sequences. Enzymes other than MseI and MluCI that cut within or adjacent to other PAM sequences are also envisaged as being within the scope of the invention. Exemplary, but non-limiting examples of restriction enzymes that produce digested fragments with terminal PAM sequences are listed in Table 2. MseI or MluCI digested DNA fragments are then treated with mung bean nuclease to degrade the single stranded overhangs (303, 304, 305). Adapters comprising MmeI and FokI restriction sites are then ligated to these DNA fragments. The adapter sequence will depend on whether the starting nucleic acid material was cut with MseI (306) or MluCI (307). The MmeI enzyme is then used to cut the DNA fragment 20 bp away from the MmeI site in the adapter sequence, removing unwanted DNA sequence from the 20-nucleotide nucleic acid targeting sequence (N20). Following MmeI digestion, the FokI enzyme is then used to cut adjacent to the adapter liberating the 20-nucleotide nucleic acid targeting sequence (N20) (308, 309). An additional adapter comprising a promoter sequence such as a T7 promoter sequence and a nucleic acid guided nuclease system protein binding sequence is then ligated to the DNA fragment comprising the N20 sequence (310, 311). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

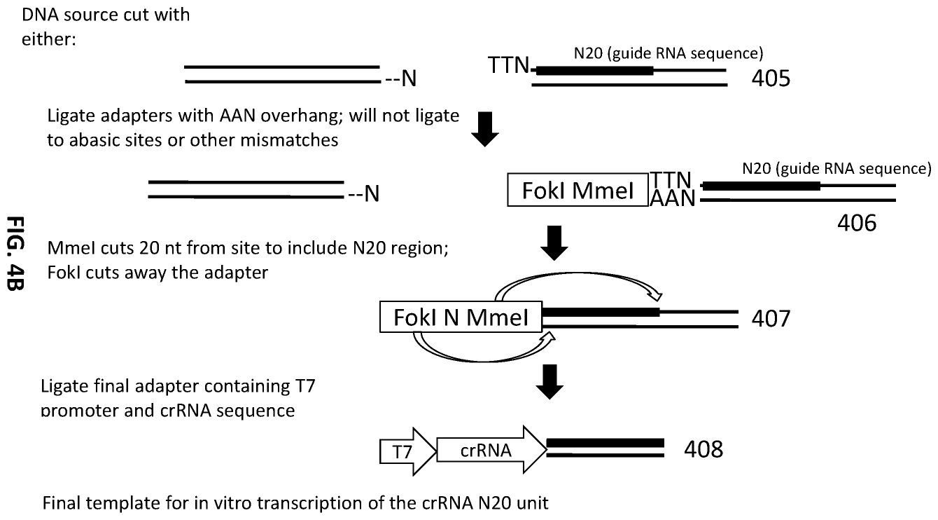

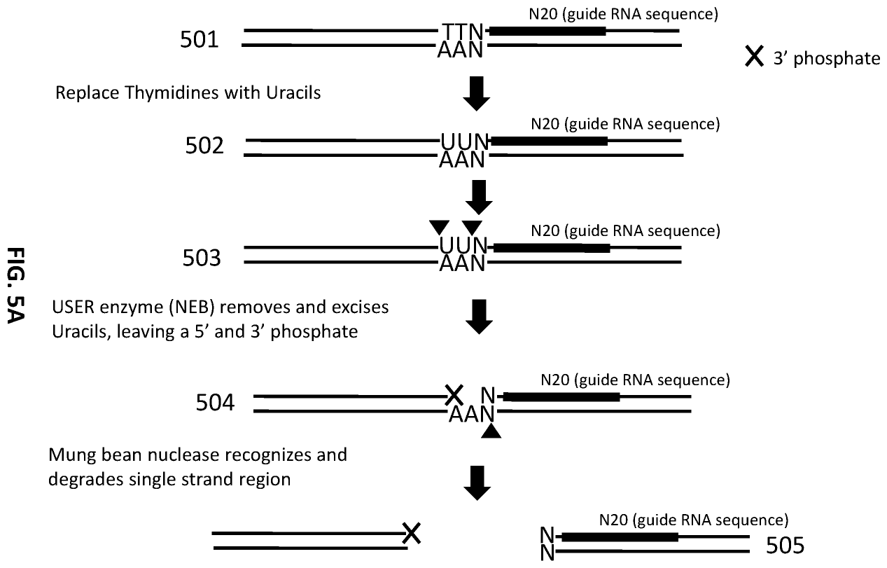

FIG. 4 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA in which the Adenines have been replaced with Inosines (FIG. 4). When Adenines have been replaced with Inosines (402), human Alkyladenine DNA Glycosylase (hAAG) is used to remove the Inosines that are based-paired with Thymines, leaving abasic sites (403). These abasic sites cannot base-pair, which causes mismatches that are recognized and cut by T7 Endonuclease I (404), resulting in DNA fragments with, for example, a TTN overhang (405). In certain embodiments, TTN functions as a PAM site. For example, Cpf1 proteins isolated from Francisella tularensis recognize TTN as a PAM. This TTN overhang can be used to ligate adapters with AAN overhangs. This overhang, in the 5′ to 3′ direction, is 5′-NAA-3′ and is complementary to the TTN overhang of DNA fragments produced by this method (406). A feature of these AAN overhang containing adapters is that these adapters will not ligate to abasic sites or other mismatches, which leads to adapter ligation specific to those N20 containing fragments that comprise TTN PAM sites as overhangs. DNA fragments, with, for example, a TNN terminal sequence that was cut by the T7 Endonuclease I of this method will fail to ligate to an adapter. This produces a collection of nucleic acid molecules comprising an adapter such as an adapter comprising FokI and MmeI restriction sites, a TTN sequence, and a nucleic acid targeting sequence (N20) (406). The MmeI restriction enzyme is then used to cut 20 bp away from the MmeI site in the adapter sequence, removing unwanted DNA sequence from the 20-nucleotide nucleic acid targeting sequence (N20). Following MmeI digestion, FokI is used to cut adjacent to the adapter, liberating the 20-nucleotide nucleic acid targeting sequence (N20) (407). An additional adapter comprising a promoter sequence such as a T7 promoter sequence and a nucleic acid guided nuclease system protein binding sequence is then ligated to the DNA fragment comprising the N20 sequence (408). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

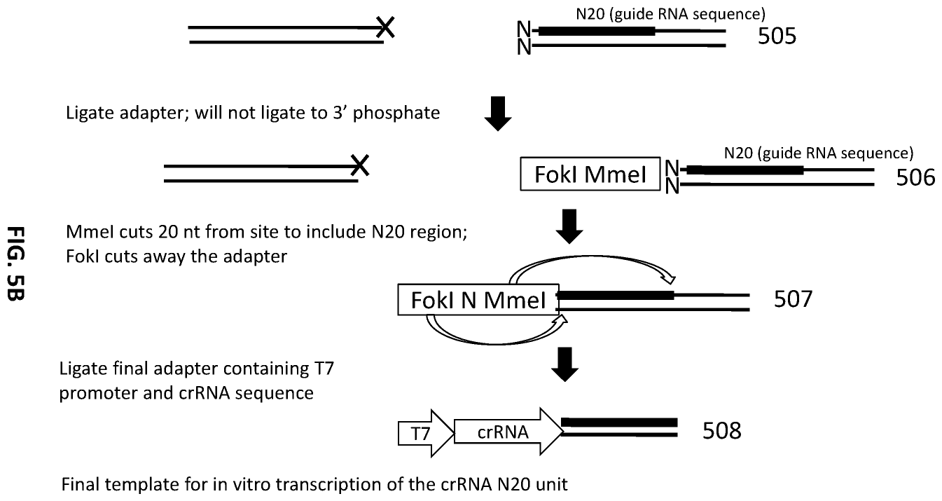

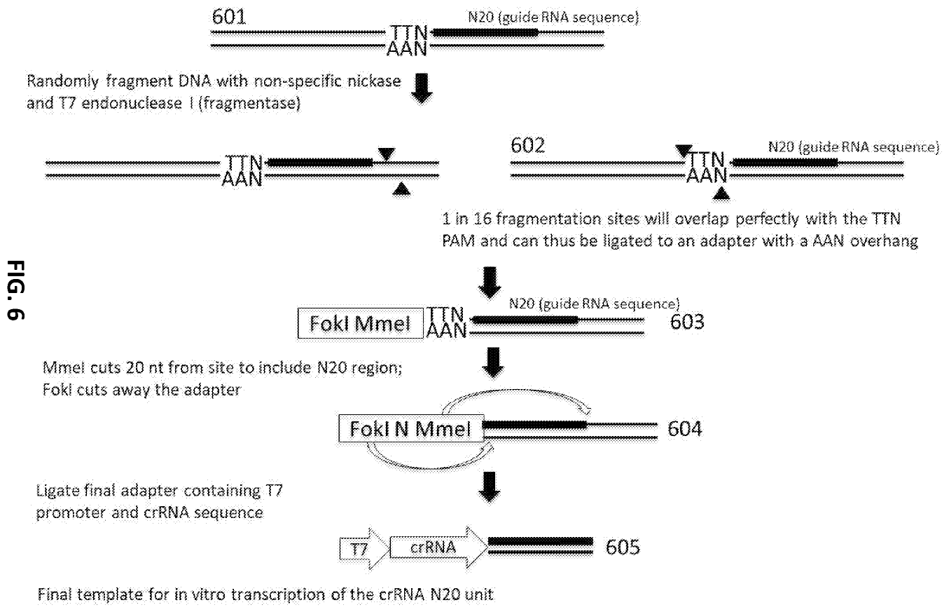

FIG. 5 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA in which the Thymidines have been replaced with Uracils (502). The USER Enzyme (Uracil-Specific Excision Reagent, NEB #M5505S) removes and excises the Uracils, leaving a 5′ and a 3′ phosphate (504). With USER, a Uracil DNA Glycosylase (UDG) catalyzes the excision of a uracil base to generate an abasic site, and Endonuclease VIII breaks the phosphodiester backbone at the 3′ and 5′ sides of the abasic site.

In certain embodiments of this method, phosphatase treatment removes the 3′ phosphate adjacent to the abasic site, followed by a single base pair extension using the dideoxyribonucleic acid ddTTP, prior to treatment with mung bean nuclease. Other DNA repair enzymes that can produce abasic sites are envisioned as within the scope of the invention. For example, a DNA glycosylase such as human Oxoguanine glycosylase (hOGG1) can be used to excise mismatched base pairs and generate abasic sites. A feature of this method is that specificity for fragmentation of the starting DNA at TTN sites, rather than, for example TN sites, comes in part from the combination of USER mediated excision and ddTTP extension. For TN sites, the end product is a nick, which makes a poor substrate. For TTN (or greater than two Ts), there is an at least one base pair gap that is more efficiently cleaved. In an alternative embodiment, USER-mediated Uracil excision is followed immediately by mung bean nuclease degradation of the single stranded region. Mung bean nuclease then recognizes and degrades the single stranded region (505). Mung bean nuclease treatment produces a collection of DNA fragments whose 5′ end is adjacent to the TT of a TTN site. In certain embodiments, TTN functions as a PAM site. For example, Cpf1 proteins isolated from Francisella tularensis recognize TTN as a PAM. Adapters comprising FokI and MmeI sites are ligated to the resulting nucleic acid fragments (506). A feature of these adapters is that these adapters will not ligate to 3′ phosphates. The MmeI restriction enzyme is used to cut 20 bp away from the MmeI site in the adapter sequence, removing unwanted DNA sequence from the 20-nucleotide nucleic acid targeting sequence (N20), and FokI is used to cut adjacent to the adapter liberating the 20-nucleotide nucleic acid targeting sequence (N20) (507). An additional adapter comprising a promoter sequence such as a T7 promoter sequence and a nucleic acid guided nuclease system protein binding sequence is then ligated to the DNA fragment comprising the N20 sequence (508). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

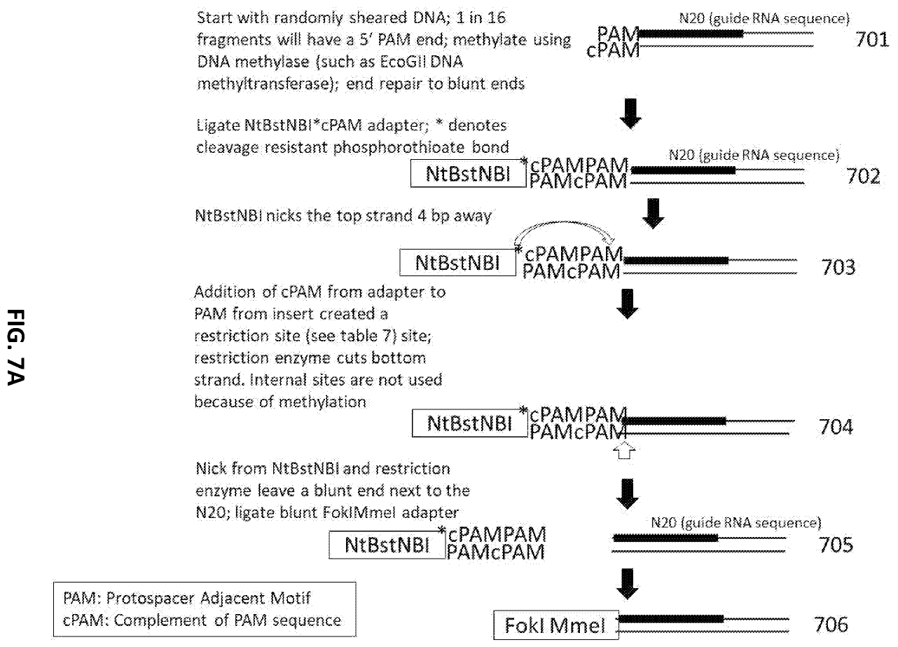

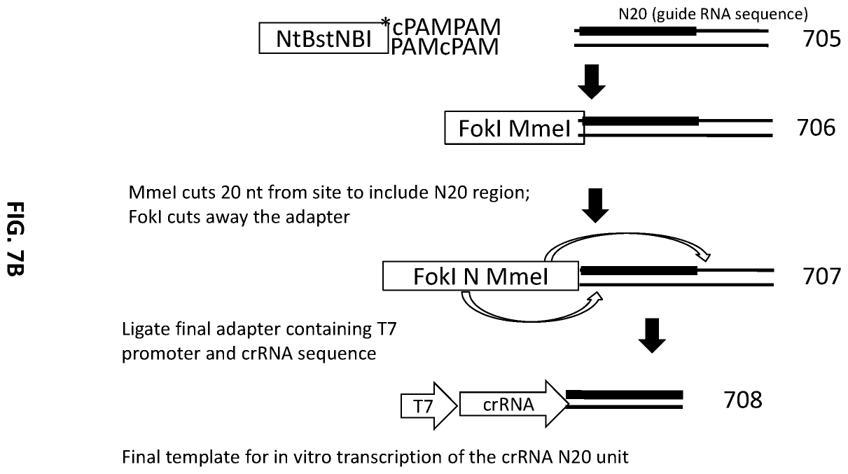

FIG. 6 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA which has been randomly fragmented with a non-specific nickase and T7 endonuclease I (fragmentase). In certain embodiments, 1 in 16 fragmentation sites will overlap perfectly with the TTN PAM site (602), producing a TTN overhang that can be ligated to an adapter comprising an AAN overhang. This produces a collection of adapter ligated DNA fragments that comprise an N20 sequence adjacent to a TTN PAM sequence. For example, an adapter comprising FokI and MmeI restriction sites is ligated to the DNA fragments (603). The MmeI enzyme is then used to cut 20 bp away from the MmeI site in the adapter sequence removing unwanted DNA sequence from the 20-nucleotide nucleic acid targeting sequence (N20), and FokI used to cut adjacent to the adapter liberating the 20-nucleotide nucleic acid targeting sequence (N20) (604). An additional adapter comprising a promoter sequence such as a T7 promoter sequence and a nucleic acid guided nuclease system protein binding sequence is then ligated to the DNA fragment comprising the N20 sequence (605). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

FIG. 7 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA which has been randomly sheared. In certain embodiments, 1 in 16 fragments will have a 5′ PAM end (701). The 5′ end of the randomly sheared DNA fragments can be methylated using a DNA methylase such as EcoGII DNA methyltransferase, and end repaired to produce blunt ends (701). An NtBstNBI*cPAM is ligated to the ends of the sheared, methylated and end repaired DNA fragments comprising the N20 nucleic acid targeting sequence (702). (*) denotes a cleavage resistant phosphorothioate bond, which negates second strand cutting. NtBstNBI (also called Nt. NstNBI) then nicks the top strand of the DNA 4 base pairs away from the phosphorothioate bond (703). In some embodiments, the NtBstNBI*cPAM adapter comprises a sequence such that the addition of the complementary PAM (cPAM) sequence of the adapter to the PAM sequence of the DNA fragment creates a restriction site (see table 2 for PAMs and the associated sequences and restriction enzymes). This restriction site can be cut by a restriction enzyme such as HaeIII, MluCI, AluI, DpnII or FatI. The creation of the restriction site through the ligation of the NtBstNBI*cPAM adapter (703) to the sheared DNA fragment comprising a PAM site, and the subsequent cleavage of the newly created restriction site (703, 704) allows for the selective processing of only those DNA fragments containing a terminal PAM sequence. The cleavage resistant phosphorothioate bond in the adapter negates second strand cutting by the restriction enzyme, and internal sites are not used because of methylation. Using an AATT PAM and MluCI as an example, by nicking the top strand at the PAM site with NtBstNBI producing an AATT (cut) position before cutting with MluCI, which cuts both strands, a blunt ended fragment is produced, as opposed to a nick or a 4 bp overhang. Only a blunt fragment can ligate to the adapter. The NtBstNBI nick (703) and the restriction enzyme cut produce a blunt end next to the N20 sequence (705), to which an adapter comprising a FokI site and an MmeI site is ligated (706). The MmeI enzyme then cuts 20 bp away from the adapter sequence removing unwanted DNA sequence from the 20-nucleotide nucleic acid targeting sequence (N20), and FokI cuts adjacent to the adapter liberating the 20-nucleotide nucleic acid targeting sequence (N20) (707). An additional adapter comprising a promoter sequence such as a T7 promoter sequence and a nucleic acid guided nuclease system protein binding sequence is then ligated to the DNA fragment comprising the N20 sequence (708). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

TABLE 2

| | | Restriction |
|---|---|---|
| | | enzyme to be |
| Target | Sequence of | utilized to |
| sequence | initial adapter | specifically |
| and | (PBS = primer | cut terminal |
| PAM | binding site) | PAM sites |
| N20-NGG | PBS-GAGTCGG (NtBstNBI | HaeIII |
| | Ad) Circ-GG (Circ Ad) | |
| TTN-N20 | PBS-GAGTCAA (NtBstNBI | MluCI |
| | Ad) Circ-AA (Circ Ad) | |
| N20-NAG | PBS-GAGTCAG (NtBstNBI | AluI |
| | Ad) Circ-AG (Circ Ad) | |
| TCN-N20 | PBS-GAGTCGA (NtBstNBI | DpnII |
| | Ad) Circ-GA (Circ Ad) | |
| TGN-N20 | PBS-GAGTCCA (NtBstNBI | FatI |
| | Ad) Circ-CA (Circ Ad) | |

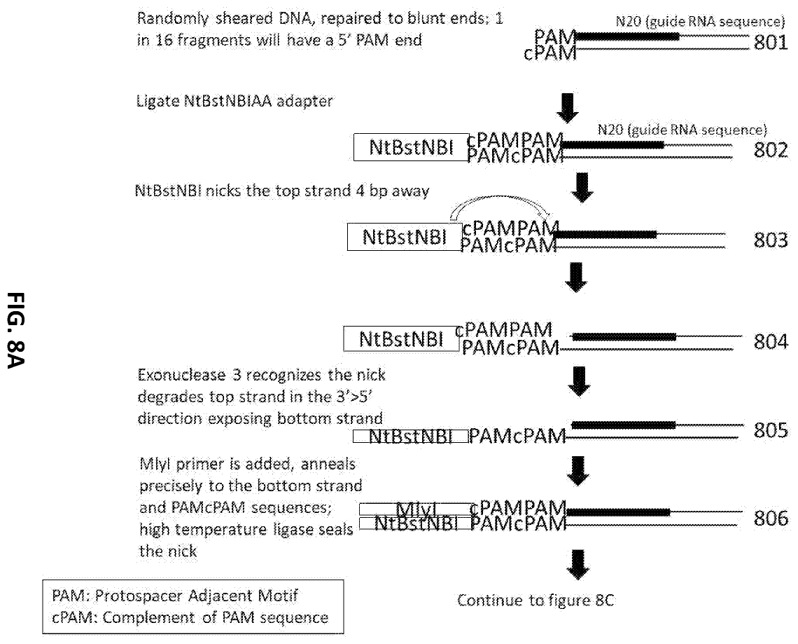

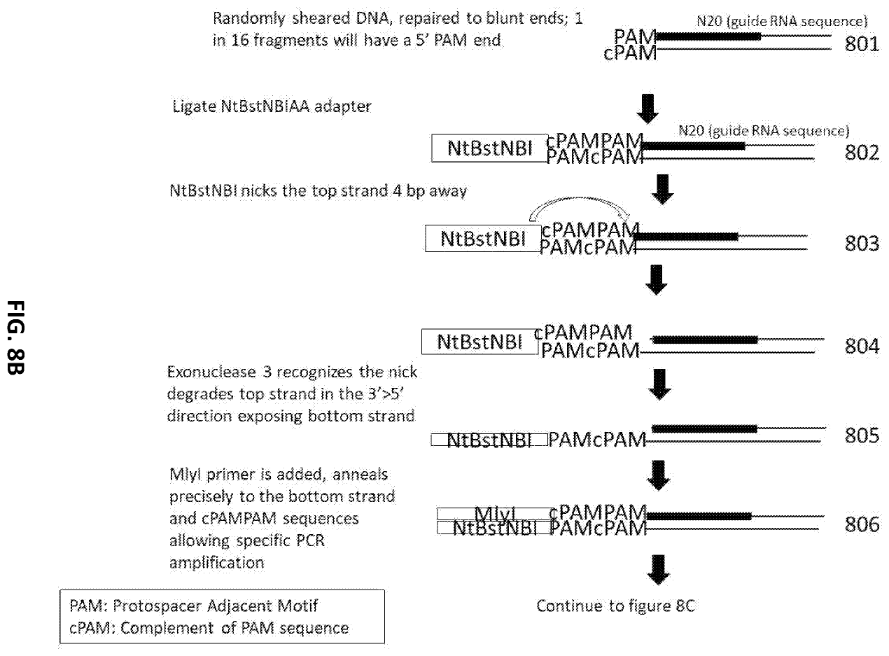

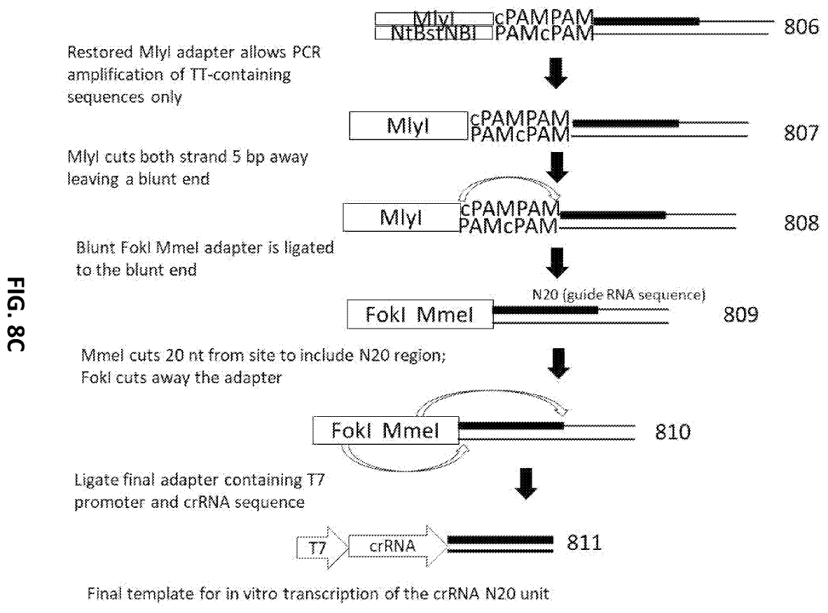

FIG. 8 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA which has been randomly sheared and repaired to blunt ends. In certain embodiments, 1 in 16 fragments will have a 5′ PAM end (801, PAM and complementary PAM (cPAM) sequences, as indicated). An NtBstNBIAA adapter is ligated to the randomly sheared, blunt ended DNA fragments (802), and NtBstNBI then nicks the top strand 4 base pairs away (803). Exonuclease 3 recognizes the nick (804) and degrades the top strand in the 3′ to 5′ direction exposing the bottom strand (805). An MlyI primer is added which anneals precisely to the bottom strand and the PAMcPAM sequences. A high temperature ligase seals the nick (806) which creates specificity for only those sheared, blunted DNA fragments comprising a terminal PAM sequence, and which gave rise to an PAMcPAM sequence upon ligation of the NtBstNBI adapter. Only creation of the PAMcPAM sequence allows precise ligation. Any other fragments will have a mismatch near the ligation site and this will negate the activity of the ligase. In some embodiments, the restored MlyI adapter allows for selective PCR amplification of the TT-containing sequences only of 806 (FIG. 8B) producing the MlyI fragments of 807, i.e. PCR amplified DNA fragments that contain both an MlyI sequence and PAM adjacent N20 sequences. PCR amplification is carried out with an enzyme without proofreading 3′ to 5′ exonuclease activity. MlyI then cuts both strands 5 base pairs away, leaving a blunt end and removing the PAMcPAM sequence (808). A blunt adapter comprising FokI and MmeI restriction sites is then ligated to the MlyI digested DNA fragments (809). The MmeI enzyme then cuts 20 bp away from the adapter sequence removing unwanted DNA sequence from the 20-nucleotide nucleic acid targeting sequence (N20), and FokI cuts adjacent to the adapter liberating the 20-nucleotide nucleic acid targeting sequence (N20) (810). An additional adapter comprising a promoter sequence such as a T7 promoter sequence and a nucleic acid guided nuclease system protein binding sequence is then ligated to the DNA fragment comprising the N20 sequence (811). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

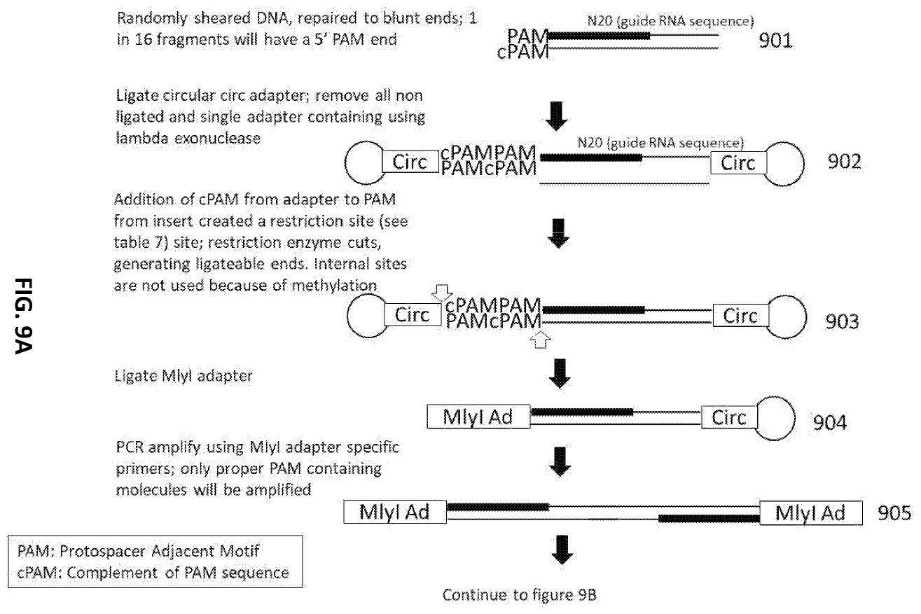

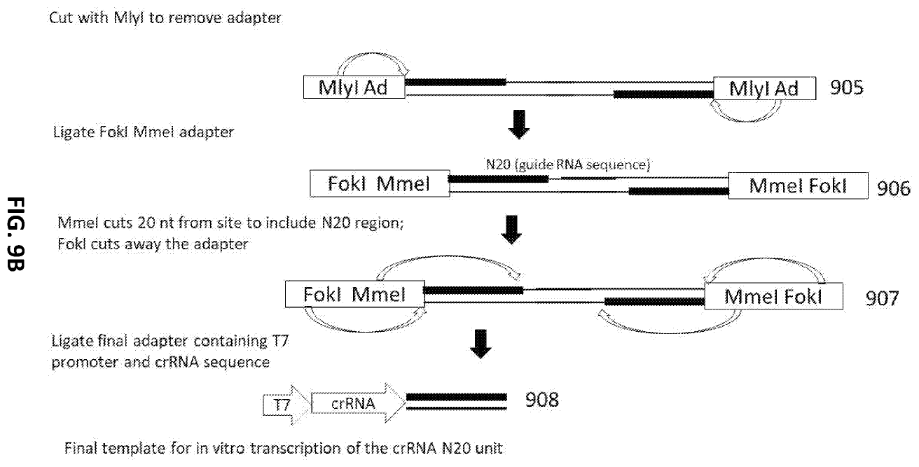

FIG. 9 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA which has been randomly sheared and repaired to have blunt ends. In certain embodiments, 1 in 16 fragments will have a 5′ PAM end (901, PAM and complimentary PAM (cPAM), as indicated). A circular adapter (circ adapter) is ligated to these blunt ended DNA fragments, and fragments without circular adapters at both ends are degraded using lambda exonuclease (902). In some embodiments, the addition of the cPAM sequence from the adapter to the PAM sequence of the DNA fragment creates a restriction site (see Table 2, and 903). This restriction site can be cut by a restriction enzyme such as HaeIII, MluCI, AluI, DpnII or FatI. When this site is cut by a restriction enzyme such as HaeIII, MluCI, AluI, DpnII or FatI, it generates ligate-able ends. The creation of the restriction site through the ligation of the circular adapter (902 to the sheared DNA fragment comprising a PAM site, and the subsequent cleavage of the newly created restriction site (903) allows for the selective processing of only those DNA fragments containing a terminal PAM sequence. Fragments with adapters that are not ligated at the PAM site will not be cut by the restriction enzyme (e.g. MluCI) at this step, and will thus remain circular. These circular fragments are unavailable for the subsequent rounds of ligation. Only the fragments with adapters ligated at the PAM sites will resist lambda nuclease (902), and then be cut by the restriction enzyme (e.g. MluCI, and 903) thus opening them for the subsequent ligation round. Internal restriction sites are not used because of methylation. A methyltransferase such as EcoGII can be used as a pre-treatment. An additional adapter comprising an MlyI sequence is then ligated to the DNA fragments (904). The DNA fragments are PCR amplified using MlyI adapter specific PCR primers (905). Only DNA molecules containing proper PAM sequences will be amplified. The amplified PCR product is then cut with MlyI to remove the adapter (FIG. 9B, 905), and an adapter comprising FokI and MmeI restriction sites is ligated to the resulting DNA fragment (906). The MmeI enzyme then cuts 20 bp away from the adapter sequence removing unwanted DNA sequence from the 20-nucleotide nucleic acid targeting sequence (N20), and FokI cuts adjacent to the adapter liberating the 20-nucleotide nucleic acid targeting sequence (N20) (907). An additional adapter comprising a promoter sequence such as a T7 promoter sequence and a nucleic acid guided nuclease system protein binding sequence is then ligated to the DNA fragment comprising the N20 sequence (908). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

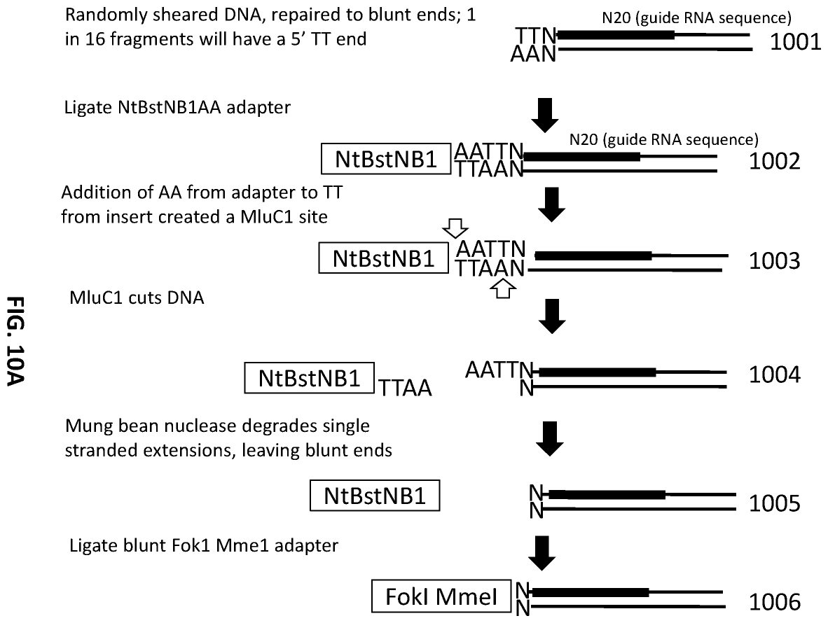

FIG. 10 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA which has been randomly sheared and repaired to have blunt ends. In certain embodiments, 1 in 16 fragments will have a 5′ TT end (1001, TTN and AAN, as indicated). In certain embodiments, TTN can be used as a PAM site. For example, TTN is recognized by Cpf1 and related family members. An NtBstNBI adapter comprising terminal an AA (NtBstNBIAA) is then ligated to the TT end (1002). The addition of 3′ terminal AA from the adapter to 5′ terminal TT from the DNA fragment creates an MluCI restriction site. MluCI cuts in this newly created site (1003), leaving an AATT single stranded overhang (1004), which is degraded by mung bean nuclease to leave blunt ended fragments (1005). The creation of the AATT MluCI restriction site by the ligation of the NtBstNBI adapter with a terminal AA to sheared DNA fragments with a terminal TT allows for the selective processing of N20 DNA fragments adjacent to a TTN PAM sequence. An adapter comprising FokI and MmeI restriction sites is ligated to the resulting DNA fragment (1006). This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

Alternatively, following ligation of the NtBstNBI adapter, NtBstNBI may be used to nick the top strand 4 base pairs away (1007), and MluCI used to cut the top and bottom strand (1008). The nick from the NtBstNBI and the cut from the MluCI produce a blunt end next to the N20 sequence (1009), to which a blunt ended adapter comprising FokI and MmeI restriction sites is ligated (1010). In certain embodiments, the NtBstNBI adapter may be an NtBstNBI*AA adapter, where (*) denotes a cleavage resistant phosphorothioate bond (1011). NtBstNBI is used to nick the top strand 4 base pairs away (1012). The addition of AA from the adapter to TT from the DNA fragment creates an MluCI restriction site, and MluCI cuts the bottom strand of this restriction site (1013). The nick from NtBstNBI and the cut from the MluCI produce a blunt end next to the N20 sequence (1014), to which a blunt ended adapter comprising FokI and MmeI restriction sites is ligated (1015). After the blunt ended adapter comprising FokI and MmeI restriction sites has been ligated to the DNA fragments comprising the N20 sequence, the MmeI enzyme then cuts 20 bp away from the adapter sequence removing unwanted DNA sequence from the 20-nucleotide nucleic acid targeting sequence (N20), and FokI cuts adjacent to the adapter liberating the 20-nucleotide nucleic acid targeting sequence (N20) (1016). An additional adapter comprising a promoter sequence such as a T7 promoter sequence and the crRNA sequence is then ligated to the DNA fragment comprising the N20 sequence (1017). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

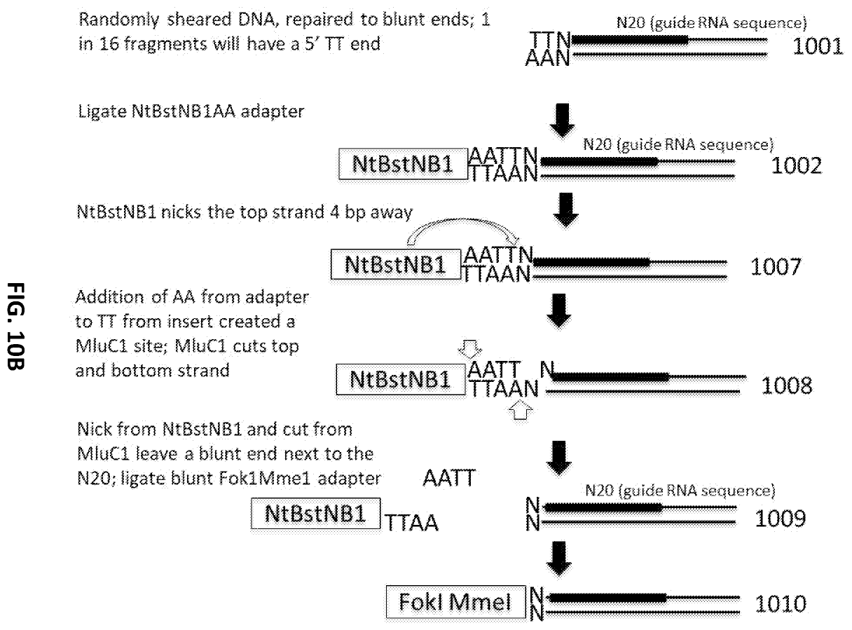

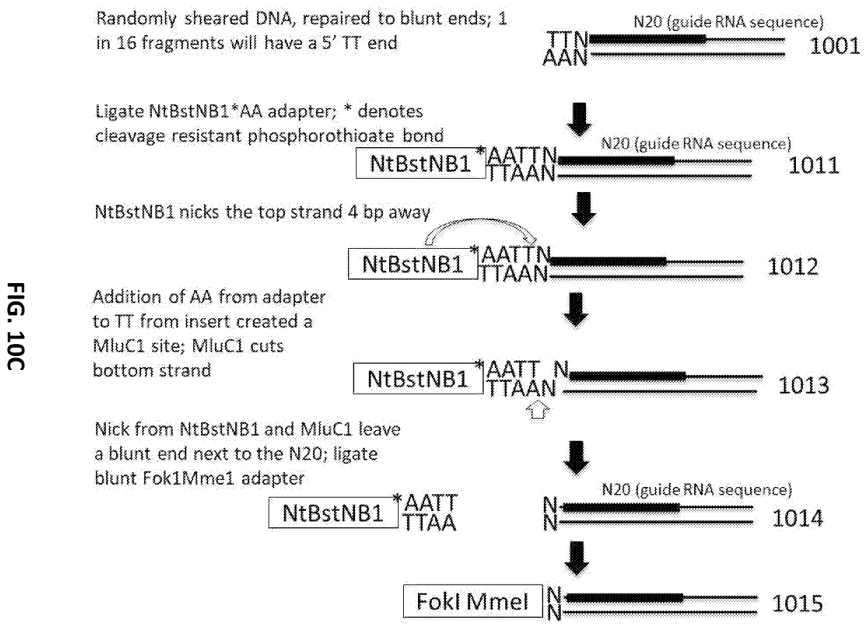

FIG. 11 shows an additional technique for constructing a gRNA library from input nucleic acids (e.g., DNA), such as genomic DNA (e.g., human genomic DNA, reverse transcribed cDNA such as from mRNA). In certain embodiments, the nucleic acid starting material for constructing a gRNA library comprises DNA which has been randomly sheared and repaired to have blunt ends. In certain embodiments, 1 in 16 fragments will have a 5′ TT end (1101, TTN and AAN, as indicated). In certain embodiments, TTN can be used as a PAM site. For example, Cpf1 proteins isolated from Francisella tularensis recognize TTN as a PAM. The NtBstNBI adapter comprising a terminal AA (NtBstNBIAA) is ligated to the end of the sheared, blunted DNA fragment (1102). When the sheared blunted DNA fragment comprises a terminal TT, ligation of the NtBstNBI adapter creates an AATT sequence (1102). The NtBstNBI enzyme is used to nick the top strand 4 base pairs away (1103). Exonuclease 3 recognizes the nick and degrades the top strand in the 3′ to 5′ direction, exposing the bottom strand (1105). An MlyI primer is added which anneals precisely to the bottom strand and the AATT sequence (1106). A high temperature ligase seals the nick (FIG. 11A, 1106), which creates specificity for only those sheared, blunted DNA fragments comprising a terminal TT sequence, and which gave rise to an AATT sequence upon ligation of the NtBstNBI AA adapter. In some embodiments, the restored MlyI adapter allows PCR selective amplification of the AATT-containing DNA fragments, i.e. those with TTN PAM adjacent N20 sequences (1107, FIG. 11B). MlyI then cuts both strands 5 base pairs away, leaving a blunt end and removing the AATT sequence (1108). A blunt adapter comprising FokI and MmeI restriction sites is then ligated to the MlyI digested DNA fragments (1109). The MmeI enzyme then cuts 20 bp away from the adapter sequence removing unwanted DNA sequence from the 20-nucleotide nucleic acid targeting sequence (N20), and FokI cuts adjacent to the adapter, liberating the 20-nucleotide nucleic acid targeting sequence (N20) (1110). An additional adapter comprising a promoter sequence such as a T7 promoter sequence and a nucleic acid guided nuclease system protein binding sequence is then ligated to the DNA fragment comprising the N20 sequence (1111). This produces the final template for in vitro transcription of the crRNA N20 unit to produce a gRNA. This method is presented with reference to generating gRNAs with 20-base pair targeting sequences; it can be modified to yield targeting sequences with other lengths, for example by adjusting the spacing between a restriction enzyme site and the targeting sequence such that the restriction enzyme cuts to yield a different length targeting sequence.

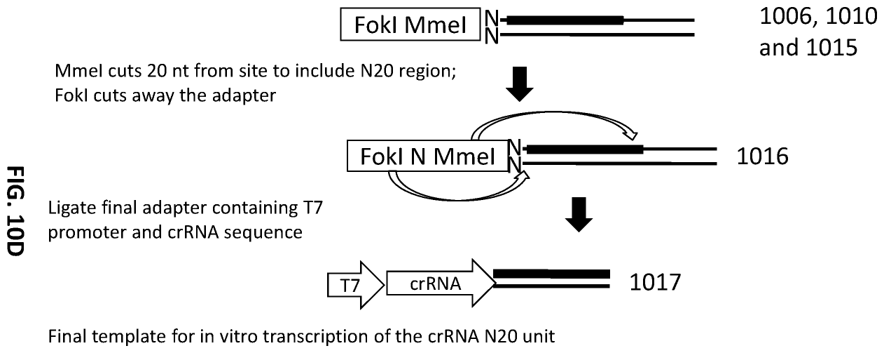

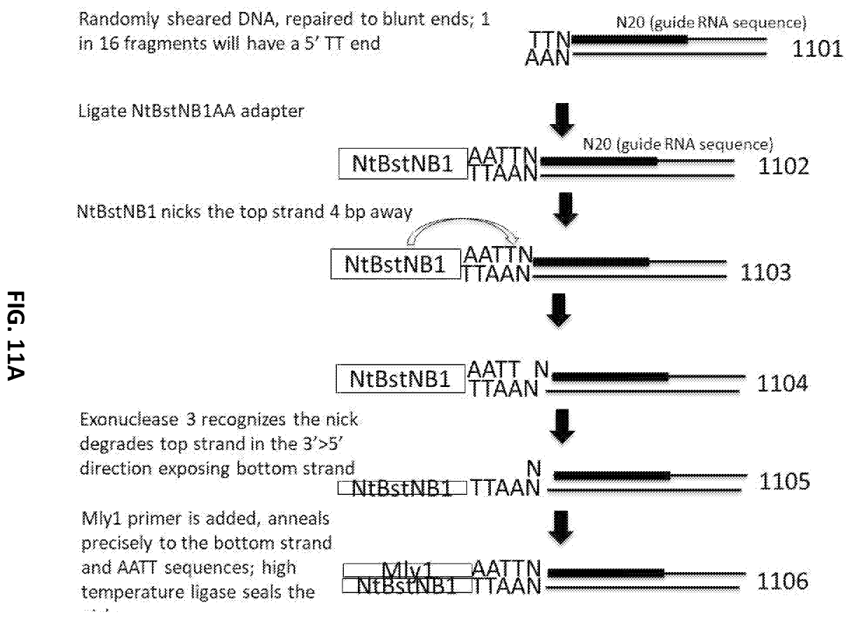

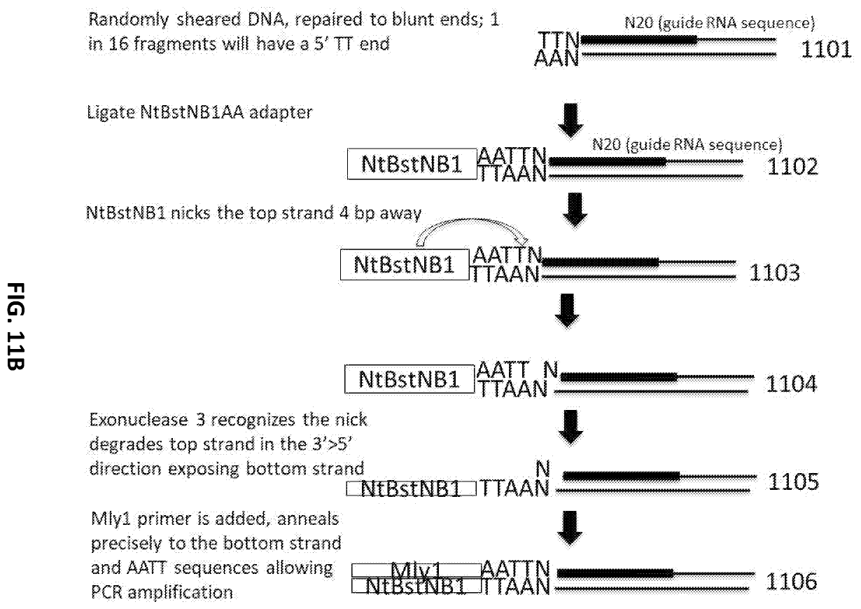

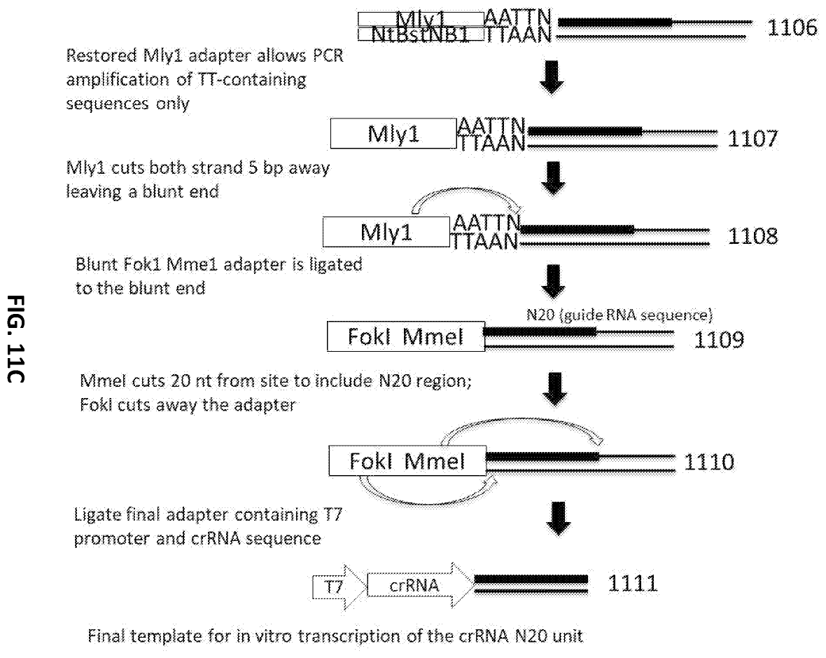

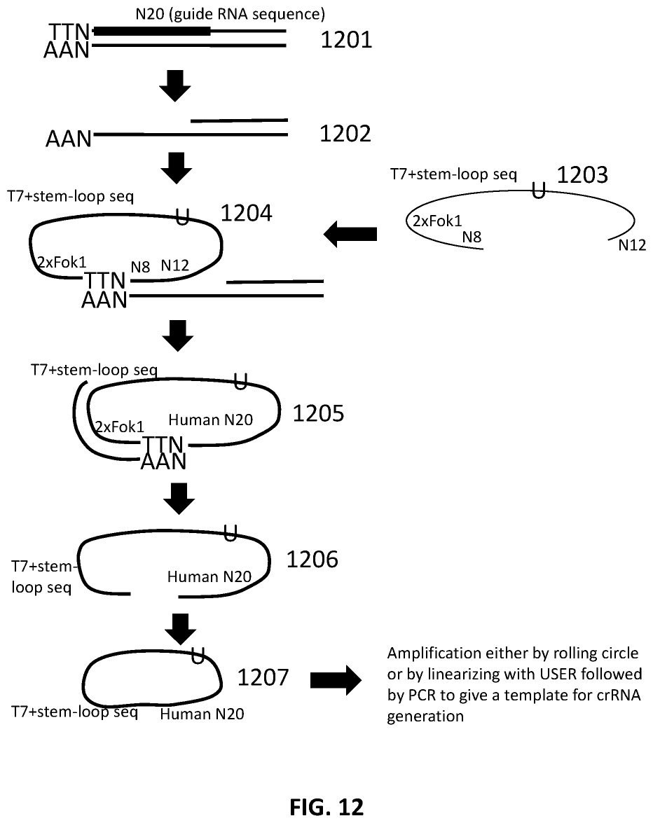

FIG. 12 shows an additional technique for constructing a gRNA library from input nucleic acids (e.g., DNA), such as genomic DNA (e.g., human genomic DNA, reverse transcribed cDNA such as from mRNA). A feature of the method is the ligation at high temperature, that results in circularization of the oligo, and converts randomized N20 sequences to N20 repertoires, as well as building a library of crRNA molecules. In certain embodiments, the nucleic acid starting material for constructing a gRNA library comprises DNA which has been randomly sheared and repaired to have blunt ends. In certain embodiments, 1 in 16 fragments will have a 5′ TT end (1201, TTN and AAN, as indicated). The double stranded DNA fragments are treated with T7 exonuclease to expose a single strand (1202). Following treatment with T7 exonuclease, a linear oligo comprising a 5′ phosphate, a random N12 sequence at the 5′ end, a T7+ stem-loop sequence, 2 opposed Fok1 sites and a TTN sequence followed by an N8 sequence at the 3′ (1203) is added, annealed to the exposed single stranded DNA, and ligated using HiFidelity Taq ligase (1204). High temperature ligase requires greater than 10 bp perfect homology on either side of the nick to ligate. If there is less homology, gaps or mismatches, it will not ligate. This produces a circularized product, and thus the random nucleotides (N8+N12) form a library of N20 sequences adjacent to a TTN PAM site (for example, a library of human N20 sequences as shown in FIG. 12). All remaining DNA is degraded using Exonuclease 1 and Exonuclease 3. An oligo complementary to the 2 opposed Fok1 regions is annealed to the circular DNA (1205) and the resulting product is cut with Fok1. This excises the (double stranded) opposed FokI sites, producing a collection of linear single stranded DNA fragments. TTN and unwanted sequences between end of stem-loop and N20 are eliminated (1206). These DNA fragments are self-circularized using CircLigase (a single stranded DNA ligase, Lucigen) (1207). The resulting circular DNAs are then amplification either by rolling circle amplification or by linearizing with USER followed by PCR to give a template for crRNA (gRNA) generation. This method is presented with reference to generating gRNAs with 20-base pair targeting sequences; it can be modified to yield targeting sequences with other lengths, for example by adjusting the lengths of the N12 and/or N8 sequences to yield a different length targeting sequence.

### Design and Synthesis

Collections of guide nucleic acids can be designed (e.g., computationally) and then synthesized for use. For example, collections of gRNAs with a 5′ protein binding sequence (stem loop) compatible with a Cpf1 system protein and a 3′ targeting sequence can be designed and synthesized. Synthesis of gRNAs can employ standard oligonucleotide synthesis techniques. In some cases, precursors to the gRNAs can be synthesized, from which the gRNAs can be produced. In an example, DNA precursors are synthesized, and gRNAs are transcribed (e.g., via in vitro transcription) from the DNA precursors. Following in vitro transcription, additional untemplated 3′ nucleotides can be removed using the methods of the disclosure.

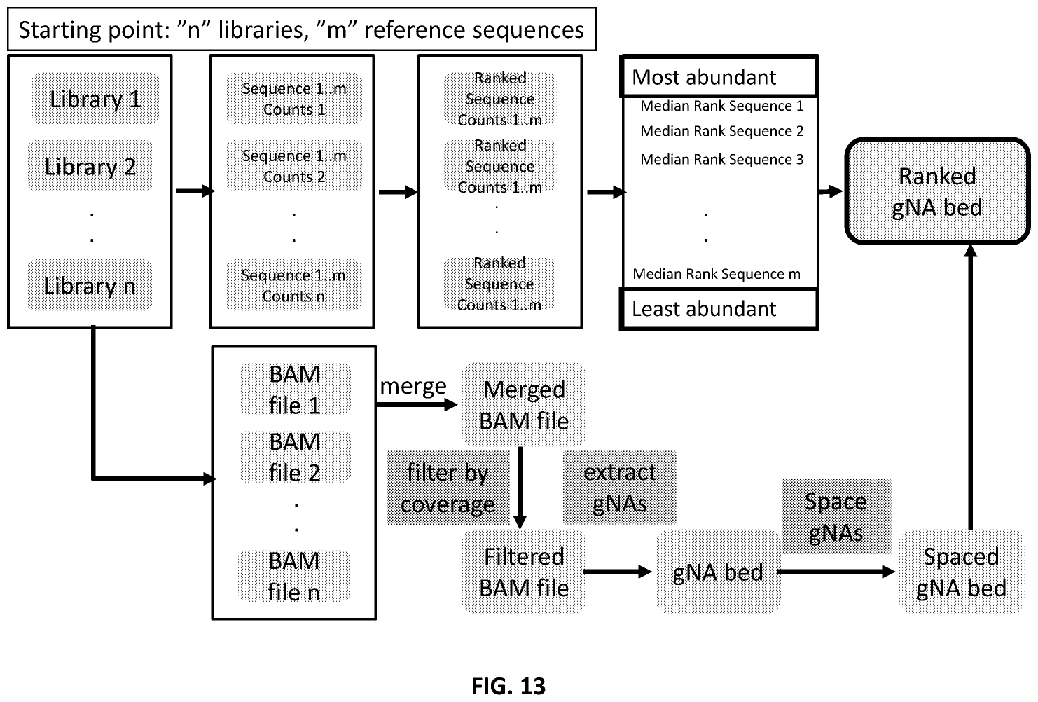

FIG. 13 illustrates a technique for designing collections of guide nucleic acids. Sequence information for the target nucleic acid sequences (e.g., target genome, target transcriptome) can be obtained. Multiple sequencing libraries can be created that include the target nucleic acid, these libraries can be sequenced to the desired coverage, and raw sequencing read data can be generated. Reads from each sequenced library can be mapped to suitable reference sequence(s). Considering all reads that reliably map to the reference sequence(s), a sequence read alignment file (e.g., binary read alignment or “BAM” file) can be created, and the number of target reads that originated from a given reference sequence (the “abundance”) can be calculated. The abundance measures obtained per target sequence can be sorted in decreasing order. Files from multiple sequencing libraries can be merged to create a single file. Regions of the sequence alignment (herein “target regions”) that are covered by a minimum number of reads can be identified. Guide nucleic acid sequences (e.g., 20 nucleotides immediately following a “TTN” motif or other PAM site on either DNA strand) can be extracted from target regions. Next, an additional filtration step can be performed to ensure that gRNAs are spaced by a minimum number of nucleotides. Map reads from each sequenced library to suitable reference sequence(s). This approach can give weight to more abundant sequences in the target sequences (e.g., cDNA from more abundant mRNA molecules for a transcriptome). For example, if the sequencing reads are from cDNA, then the number of reads can be correlated with the abundance of the associated transcript.

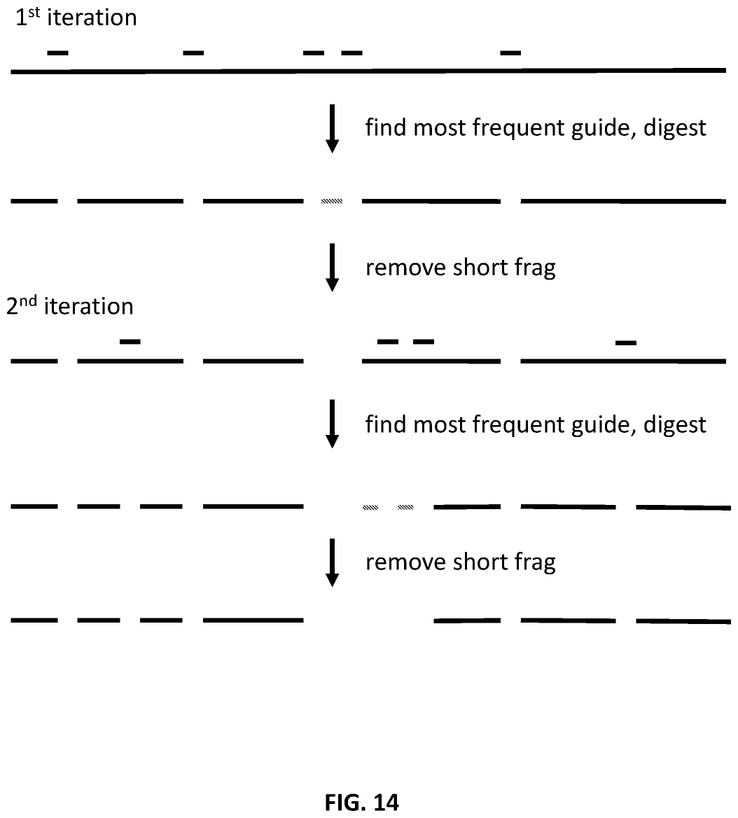

FIG. 14 illustrates a technique for designing collections of guide nucleic acids. Sequence information for the target nucleic acid sequences (e.g., target genome, target transcriptome) can be obtained. The most frequent guide nucleic acid recognition sequence (aka targeting sequence) (e.g., 20 nucleotides (N20) (or other desired targeting region length) immediately following a “TTN” motif or other PAM site on either DNA strand) can be extracted from target regions, and a digestion can be conducted or simulated using this most frequent guide. Short fragments can be removed, and the second most frequent guide can be found and used for a digestion. Short fragments can again be removed, and the third most frequent guide can be found and used for a digestion. This process can be iterated until the number of guides matches a preset number (e.g., a preset number determined by the capacity of a synthesis method such as an array), all remaining fragments are short, no guides can be found, or an acceptable amount of digestion or depletion is enabled by the guides found. This process can be conducted computationally, locating guides and simulating digestions on the target nucleic acid sequences. Multiple guides can be found in a given iteration. For example, each iteration can yield fewer potential guides, so in some after a few iterations multiple guides can found in a given iteration. In some cases, rather than determining the most frequent guide in an iteration, the guide identified is that which yields the most fragments below a certain threshold (e.g., short fragments) after cutting. This approach can give weight to more abundant sequences in the target sequences (e.g., cDNA from more abundant mRNA molecules for a transcriptome).

Short fragments can be nucleic acids less than about 10000 bp, 9000 bp, 8000 bp, 7000 bp, 6000 bp, 5000 bp, 4000 bp, 3000 bp, 2000 bp, 1000 bp, 500 bp, 450 bp, 400 bp, 350 bp, 300 bp, 250 bp, 200 bp, 150 bp, 100 bp, 90 bp, 80 bp, 70 bp, 60 bp, 50 bp, 40 bp, 30 bp, 20 bp, or 10 bp. The preset number of guides can be at least about 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, or 10000000. The acceptable amount of depletion can be at least about 10%, 20%, 30%, 40%, 50%, 60%, 70%, 80%, 90%, 95%, 99%, 99.9%, 99.99%, 99.999%, or 100%. The amount of depletion can, in some cases, be the percentage of starting target nucleic acids that are cleaved to short fragments.

### Exemplary Compositions

In one embodiment, provided herein is a composition comprising a nucleic acid fragment, a nickase nucleic acid-guided nuclease-gRNA complex, and labeled nucleotides. In one exemplary embodiment, provided herein is a composition comprising a nucleic acid fragment, a nickase Cas9-gRNA complex, and labeled nucleotides. In such embodiments, the nucleic acid may comprise DNA. The nucleotides can be labeled, for example with biotin. The nucleotides can be part of an antibody-conjugate pair.

In one embodiment, provided herein is a composition comprising a nucleic acid fragment and a catalytically dead nucleic acid-guided nuclease-gRNA complex, wherein the catalytically dead nucleic acid-guided nuclease is fused to a transposase. In one exemplary embodiment, provided herein is a composition comprising a DNA fragment and a dCpf1-gRNA complex, wherein the dCpf1 is fused to a transposase.

In one embodiment, provided herein is a composition comprising a nucleic acid fragment comprising methylated nucleotides, a nickase nucleic acid-guided nuclease-gRNA complex, and unmethylated nucleotides. In an exemplary embodiment, provided herein is a composition comprising a DNA fragment comprising methylated nucleotides, a nickase Cpf1-gRRNA complex, and unmethylated nucleotides.

In one embodiment, provided herein is a gRNA complexed with a nucleic acid-guided-DNA endonuclease.

In one embodiment, provided herein is a gRNA complexed with a nucleic acid-guided-RNA endonuclease. In one embodiment, the nucleic acid-guided-RNA endonuclease comprises C2c2.

In one embodiment, provided herein is a collection of gRNAs produced or designed by the methods of the present disclosure.

### Samples

The methods described herein can be used to prepare a library of nucleic acids from nucleic acids isolated any biological sample.

In some embodiments, the sample is a clinical sample. In some embodiments, the sample comprises host and non-host nucleic acids, for example a human clinical sample comprising human nucleic acids and nucleic acids from one or more viruses, bacteria, fungi or eukaryotic pathogens.

In some embodiments, the sample is a forensic sample. For example, the sample can be a sample of biological material collected at a crime scene, or collected from a suspect, victim or other target. Any type of biological material from which nucleic acids can be isolated is envisaged as within the scope of the disclosure. Exemplary biological samples include blood, serum, tissue, nails (e.g., fingernails and toenails), saliva, sputum, mucus, tears, semen, vaginal excretions, hair (including hair with roots or follicles, and rootless hair shafts), cells, feces and urine.

In some embodiments, the sample is a trace sample. Trace samples are minute biological samples, for example “touch” samples that are left when a subject touches an object, such as skin cells.

In some embodiments, the sample is degraded. In some embodiments, the sample comprises small nucleic acid fragments, for example, less than about 50 base pairs.

In some embodiments, the sample comprises cell-free nucleic acids, such as cell-free DNA or cell-free RNA.

### Kits and Articles of Manufacture

The present application provides kits comprising any one or more of the compositions described herein, not limited to adapters, gRNAs, gRNA collections, nucleic acid molecules encoding the gRNA collections, and the like.

In exemplary embodiments, the kit comprises a first adapter, a second adapter, indexing primers, enzymes, control samples and instructions for use in preparing libraries from nucleic acid samples using the methods described herein. In some embodiments, the nucleic acids samples are degraded or comprise small nucleic acid fragments (e.g., less than 50 bp in length).

In exemplary embodiments, the kit comprises a collection of DNA molecules capable of transcribing into a library of gRNAs wherein the gRNAs are targeted to human genomic or other sources of DNA sequences.

In one embodiment, the kit comprises a collection of gRNAs wherein the gRNAs are targeted to human genomic or other sources of DNA sequences.

In some embodiments, provided herein are kits comprising any of the collection of nucleic acids encoding gRNAs, as described herein. In some embodiments, provided herein are kits comprising any of the collection of gRNAs, as described herein.

The present application also provides all essential reagents and instructions for carrying out the methods of making the gRNAs and the collection of nucleic acids encoding gRNAs, as described herein. In some embodiments, provided herein are kits that comprise all essential reagents and instructions for carrying out the methods of making individual gRNAs and collections of gRNAs as described herein.

Also provided herein is computer software monitoring the information before and after contacting a sample with a gRNA collection produced herein. In one exemplary embodiment, the software can compute and report the abundance of non-target sequence in the sample before and after providing gRNA collection to ensure no off-target targeting occurs, and wherein the software can check the efficacy of targeted-depletion/encrichment/capture/partitioning/labeling/regulation/editing by comparing the abundance of the target sequence before and after providing gRNA collection to the sample.

### Enumerated Embodiments

The invention may be defined by reference to the following enumerated, illustrative embodiments:
1. A method of preparing a library of nucleic acids, comprising:
a. providing a sample of nucleic acids comprising at least one sequence of interest;
b. contacting the sample of nucleic acids, a plurality of first polymerase chain reaction (PCR) primers, and a polymerase under conditions that allow PCR to occur, thereby generating a plurality of first single-sided PCR products;
c. contacting the plurality of first single-sided PCR products with a terminal transferase and dNTPs under conditions sufficient to transfer dNTPs to the 3′ ends of the plurality of first single-sided PCR products, thereby generating a plurality of PCR products comprising 3′ tails; and
d. contacting the plurality of PCR products comprising 3′ tails, a plurality of second PCR primers, and a polymerase under conditions that allow PCR to occur;
   thereby generating a library of nucleic acids with adapters at the 5′ and 3′ ends.
2. The method of embodiment 1, comprising:
e. contacting the plurality of PCR products from (d) with a plurality of first indexing primers, a plurality of second indexing primers, and a polymerase under conditions that allow PCR to occur.
3. The method of embodiment 1 or 2, wherein the plurality of first PCR primers comprise (i) a sequence complementary to a sequence adjacent to or overlapping the at least one sequence of interest, and (ii) a first adapter sequence.
4. The method of embodiment 3, wherein the first adapter sequence is 5′ of the sequence complementary to the sequence adjacent to the at least one sequence of interest.
5. The method of any one of embodiments 1-4, wherein the plurality of second PCR primers comprise (i) a sequence complementary to the 3′ tails from step (c), and (ii) a second adapter sequence.
6. The method of embodiment 5, wherein the second adapter sequence is 5′ of the sequence complementary to the 3′ tail.
7. The method of any one of embodiments 1-6, wherein first indexing primers comprise a sequence complementary to the first adapter and a first unique molecular identifier sequence (UMI).
8. The method of any one of embodiments 1-7, wherein the second indexing primers comprise a sequence complementary to the second adapter and a second UMI sequence.
9. The method of any one of embodiments 1-8, wherein the 3′ tail is a poly A tail, a polyG tail, a polyC tail or a polyT tail.
10. The method of any one of embodiments 1-9, comprising contacting the sample of nucleic acids with a first enzyme prior to step (b) under conditions that allow for blunting of overhangs in the sample of nucleic acids, thereby generating a blunt-ended sample of nucleic acids.
11. The method of embodiment 10, wherein the first enzyme comprises T4 polymerase, Klenow fragment, or Mung Bean Nuclease.
12. The method of embodiment 11, comprising purifying the blunt-ended sample of nucleic acids.
13. The method of embodiment 12, wherein the purifying comprises removing unincorporated dNTPs.
14. The method of embodiment 13, wherein removing unincorporated dNTPs comprises treating with recombinant shrimp alkaline phosphatase (rSAP), purification using a column or bead-based purification.
15. The method of any one of embodiments 10-14, comprising contacting the blunt-ended sample of nucleic acids with a second enzyme under conditions that allow for the addition of dideoxynucleotides (ddNTPs) to the to the 3′ ends of the blunt ended nucleic acids in the sample, and wherein contacting the blunt-ended sample of nucleic acids with the second enzyme occurs prior to step (b).
16. The method of embodiment 15, wherein the second enzyme has 3′ to 5 exonuclease activity and polymerase activity but does not have 5′ to 3′ exonuclease activity.
17. The method of embodiment 16, wherein the second enzyme comprises a Klenow fragment.
18. The method of embodiment 17, comprising purifying the blunt-ended sample of nucleic acids after contacting the blunt-ended sample of nucleic acids with the second enzyme.
19. The method of embodiment 18, wherein the purifying comprises removing unincorporated ddNTPs.
20. The method of embodiment 19, wherein removing unincorporated ddNTPs comprises treating with recombinant shrimp alkaline phosphatase (rSAP), purification using a column, or bead-based purification.
21. The method of any one of embodiments 1-20, comprising purifying the plurality of first single-sided PCR products following step (b).
22. The method of embodiment 21, wherein the purifying comprises removing unincorporated dNTPs.
23. The method of embodiment 22, wherein removing unincorporated dNTPs comprises treating with recombinant shrimp alkaline phosphatase (rSAP), purification using a column, or bead-based purification.
24. The method of any one of embodiments 1-23, comprising purifying the plurality of first single-sided PCR products following step (b) and prior to step (c).
25. The method of embodiment 24, wherein the purifying comprises removing unincorporated dNTPs.
26. The method of embodiment 25, wherein removing unincorporated dNTPs comprises treating with recombinant shrimp alkaline phosphatase (rSAP), purification using a column, or bead-based purification.
27. The method of any one of embodiments 1-26, comprising purifying the plurality of PCR products comprising 3′ tails after step (c) and prior to step (d).
28. The method of embodiment 27, wherein the purifying comprises removing unincorporated dNTPs.
29. The method of embodiment 28, wherein removing unincorporated dNTPs comprises treating with recombinant shrimp alkaline phosphatase (rSAP), purification using a column, or bead-based purification.
30. The method of any one of embodiments 1-29, comprising purifying the plurality of PCR products from (d).
31. The method of embodiment 30, wherein the purification comprises using a column or a bead-based purification.
32. The method of any one of embodiments 1-31, wherein the nucleic acids comprise ribonucleic acids (RNAs), deoxyribonucleic acids (DNAs), or a combination thereof.
33. The methods of any one of embodiments 7-32, wherein the first unique molecular identifier sequence (UMI) comprises 2, 3, 4, 5, 6, 7, 8, 9, 10, 11 or 12 nucleotides.
34. The method of embodiment 33, wherein the first UMI is a random sequence.
35. The method of any one of embodiments 1-34, wherein the first adapter comprises a sequence of a first sequencing adapter.
36. The method of any one of embodiments 8-35, wherein the second UMI comprises 2, 3, 4, 5, 6, 7, 8, 9, 10, 11 or 12 nucleotides.
37. The method of embodiment 36, wherein the second UMI is a random sequence.
38. The method of any one of embodiments 1-37, wherein the second adapter comprises a sequence of a second sequencing adapter.
39. The method of any one of embodiments 1-38, wherein the sequence adjacent to the sequence of interest is within 1-500, 1-300, 1-200, 1-100, 1-75, 1-50 or 1-25 nucleotides of the sequence of interest.
40. The method of any one of embodiments 1-39, wherein the sequence adjacent to the sequence of interest is within 1-25 nucleotides of the sequence of interest.
41. The method of any one of embodiments 1-40, wherein the sequence of interest comprises a single nucleotide polymorphism (SNP), a miniSTR (mini short tandem repeat), a mitochondrial marker, a Y chromosome marker, a taxonomic marker, or a disease trait marker.
42. The method of embodiment 41, wherein the disease trait marker comprises a marker for pathogenicity, virulence, resistance or strain identification.
43. The method of any one of embodiments 1-42, wherein the sample is degraded.
44. The method of any one of embodiments 1-43, wherein the sample is a forensics sample.
45. The method of any one of embodiments 1-44, comprising sequencing the library of nucleic acids.
46. The methods of any one of embodiments 1-45, wherein the at least one sequence of interest comprises at least 1, 2, 3, 4, 5, 6, 7, 8, 9, 10, 15, 20, 25, 30, 40, 50, 60, 70, 80, 90, 100, 500, 1000, 10,000, 50,000, 100,000 or 200,000 unique sequences of interest.
47. The method of any one of embodiments 1-46, comprising sequencing the library of nucleic acids.
48. The method of embodiment 47, wherein the sequencing is high-throughput sequencing.
49. The methods of any one of embodiments 1-46, comprising:
a. providing a plurality of guide nucleic acid (gNA)-CRISPR/Cas system protein complexes, wherein the gNAs are configured to hybridize to at least one sequence targeted for depletion;
b. mixing the library of nucleic acids with the plurality of gNA-CRISPR/Cas system protein complexes, wherein at least a portion of the gNA-CRISPR/Cas system protein complexes hybridize to the at least one sequence targeted for depletion; and
c. incubating the mixture to cleave the at least one sequence targeted for depletion.
50. The method of embodiment 49, comprising PCR amplifying the library of nucleic acids following step (c).
51. The method of embodiment 49 or 50, wherein the CRISPR/Cas system protein comprises Cpf1, Cas9, Cas3, Cas8a-c, Cas10, CasX, CasY, Cas13, Cas14, Cse1, Csy1, Csn2, Cas4, Csm2, Cm5 or a combination thereof.
52. The method of any one of embodiments 49-51, wherein the CRISPR/Cas system protein comprises Cas9, Cpf1 or a combination thereof.
53. The method of any one of embodiments 49-52, wherein CRISPR/Cas system protein is a Cas9 or Cpf1 nickase.
54. The method of any one of embodiments 49-53, wherein CRISPR/Cas system protein is thermostable.
55. The method of any one of embodiments 49-54, wherein the gNAs are deoxyribonucleic acid (gDNAs) or ribonucleic acids (gRNAs).
56. The method of any one of embodiments 49-55, wherein the plurality of gNAs comprise at least 2, 10, 102, 103, 104, 105 or 106 unique gNAs.
57. The method of any one of embodiments 49-56, comprising sequencing the library of nucleic acids.
58. The method of embodiment 57, wherein the sequencing is high-throughput sequencing.
59. A method of preparing a library of nucleic acids, comprising:
a. providing a sample of nucleic acids comprising at least one sequence of interest;
b. contacting the sample of nucleic acids with a terminal transferase under conditions sufficient to transfer NTPs to the 3′ end of the nucleic acids thereby generating a plurality of nucleic acids comprising 3′ tails;
c. contacting the plurality of nucleic acids comprising 3′ tails with a plurality of first adapters and a reverse transcriptase under conditions sufficient for first strand complementary DNA (cDNA) synthesis to occur, thereby generating a plurality of cDNAs, wherein the plurality of cDNAs comprise 3′ polyC sequences; and
d. contacting the plurality of cDNAs with a second adapter under conditions sufficient to allow generation of double stranded DNA from the plurality of cDNAs to generate a plurality of double stranded DNAs,
   thereby preparing a library of nucleic acids with adapters at the 5′ and 3′ ends.
60. The method of embodiment 60, wherein the plurality of first adapters comprise a sequence complementary to the 3′ tails and a first UMI sequence.
61. The method of embodiment 60 or 61, wherein the plurality of second adapters comprise a second UMI and a polyG sequence.
62. The method of any one of embodiments 59-61, wherein the nucleic acids comprise ribonucleic acids (RNAs).
63. The method of any one of embodiments 59-62, wherein the reverse transcriptase comprises Moloney Murine Leukemia Virus (MMLV) reverse transcriptase.
64. The method of embodiment 59, wherein step (d) comprises adding a polymerase.
65. The method of embodiment 64, wherein step (d) comprises PCR amplification of the plurality of double stranded DNAs.
66. The methods of any one of embodiments 60-65, wherein the first unique molecular identifier sequence (UMI) comprises 2, 3, 4, 5, 6, 7, 8, 9, 10, 11 or 12 nucleotides.
67. The method of embodiment 65, wherein the first UMI is a random sequence.
68. The method of any one of embodiments 59-67, wherein the first adapter comprises a sequence of a first sequencing adapter.
69. The method of any one of embodiments 61-68, wherein the second UMI comprises 2, 3, 4, 5, 6, 7, 8, 9, 10, 11 or 12 nucleotides.
70. The method of embodiment 69, wherein the second UMI is a random sequence.
71. The method of any one of embodiments 59-70, wherein the second adapter comprises a sequence of a second sequencing adapter.
72. The method of any one of embodiments 59-71, wherein the sequence of interest comprises a single nucleotide polymorphism (SNP), a miniSTR (mini short tandem repeat), a mitochondrial marker, a Y chromosome marker, or a disease trait marker.
73. The method of embodiment 72, wherein the disease trait marker comprises a marker for pathogenicity, virulence, resistance or strain identification.
74. The method of any one of embodiments 59-73, wherein the sample is degraded. 75. The method of any one of embodiments 59-74, wherein the sample is a forensics sample.
76. The method of any one of embodiments 59-75, wherein the at least one sequence of interest comprises at least 1, 2, 3, 4, 5, 6, 7, 8, 9, 10, 15, 20, 25, 30, 40, 50, 60, 70, 80, 90, 100, 500, 1000, 10,000, 50,000, 100,000 or 200,000 unique sequences of interest.
77. The method of any one of embodiments 59-76, wherein the sample of nucleic acids comprises ribonucleic acids (RNAs).
78. The method of embodiments 59-77, comprising sequencing the library of nucleic acids.
79. The method of embodiment 78, wherein the sequencing comprises high-throughput sequencing. 80. The methods of any one of embodiments 59-76, comprising:
a. providing a plurality of guide nucleic acid (gNA)-CRISPR/Cas system protein complexes, wherein the gNAs are configured to hybridize to at least one sequence targeted for depletion;
b. mixing the library of nucleic acids with the plurality of gNA-CRISPR/Cas system protein complexes,
wherein at least a portion of the gNA-CRISPR/Cas system protein complexes hybridize to the at least one sequence targeted for depletion; and
c. incubating the mixture to cleave the at least one sequence targeted for depletion. 81. The method of embodiment 80, comprising PCR amplifying the library of nucleic acids following step (c).
82. The method of embodiment 80 or 81, wherein the CRISPR/Cas system protein comprises Cpf1, Cas9, Cas3, Cas8a-c, Cas10, CasX, CasY, Cas13, Cas14, Cse1, Csy1, Csn2, Cas4, Csm2, Cm5 or a combination thereof.
83. The method of any one of embodiments 80-82, wherein the CRISPR/Cas system protein comprises Cas9, Cpf1 or a combination thereof.
84. The method of any one of embodiments 80-83, wherein CRISPR/Cas system protein is a Cas9 or Cpf1 nickase.
85. The method of any one of embodiments 80-84, wherein CRISPR/Cas system protein is thermostable.
86. The method of any one of embodiments 80-85, wherein the gNAs are deoxyribonucleic acids (gDNAs) or ribonucleic acids (gRNAs).
87. The method of any one of embodiments 80-86, wherein the plurality of gNAs comprise at least 2, 10, 102, 103, 104, 105 or 106 unique gNAs.
88. The method of any one of embodiments 80-87, comprising sequencing the library of nucleic acids.
89. The method of embodiment 88, wherein the sequencing is high throughput sequencing.
90. A method of making a guide ribonucleic acid (gRNA) without at least one untemplated 3′ nucleotide, comprising:
   (a) providing a deoxyribonucleic acid (DNA) comprising, from 5′ to 3:
      (i) a sequence encoding a promoter,
      (ii) a sequence encoding a stem-loop,
      (iii) a sequence encoding a targeting sequence, and
      (iv) a sequence encoding a primer binding sequence;
   (b) contacting the DNA of (a) with a polymerase to produce an RNA comprising, from 5′ to 3′, an RNA sequence encoding a stem-loop, an RNA sequence encoding a targeting sequence, an RNA sequence encoding a primer binding sequence and at least one additional untemplated nucleotide;
   (c) hybridizing the RNA of (b) to a single stranded DNA (ssDNA) comprising a sequence complementary to the sequence encoding the primer binding sequence (iv),
      wherein conditions are sufficient for the RNA of (b) and the ssDNA to form an RNA/DNA heteroduplex region; and
   (d) contacting the RNA/DNA heteroduplex region with a Ribonuclease H (RNase H) enzyme, wherein conditions are sufficient for the RNase H enzyme to hydrolyze at least one phosphodiester bond of the RNA in the RNA/DNA heteroduplex region, thereby generating a gRNA without at least one untemplated 3′ nucleotide.
91. The method of embodiment 90, wherein the DNA of (a) is a synthetic DNA.
92. The method of embodiment 90 or 91, wherein the DNA of (a) is a PCR amplification product.
93. The method of embodiment 90 or 91, wherein the DNA of (a) is a plasmid, wherein the method further comprises contacting the plasmid with a restriction enzyme prior to the transcribing step of (b), and wherein conditions are sufficient to produce a linear plasmid DNA.
94. The method of any one of embodiments 90-93, wherein the sequence encoding the promoter is selected from the group consisting of a sequence encoding a T7 promoter, a sequence encoding an SP6 promoter or a sequence encoding a T3 promoter. 95. The method of embodiment 94, wherein the sequence encoding the T7 promoter comprises a sequence of 5′-TAATACGACTCACTATAGG-3′ (SEQ ID NO: 1).
96. The method of embodiment 95, wherein the polymerase is a T7 polymerase. 97. The method of embodiment 94, wherein the sequence encoding the SP6 promoter comprises a sequence of 5′-CATACGATTTAGGTGACACTATAG-3′ (SEQ ID NO: 5). 98. The method of embodiment 97, wherein the polymerase is an SP6 polymerase. 99. The method of embodiment 94, wherein the sequence encoding the T3 promoter comprises a sequence of 5′-AATTAACCCTCACTAAAG-3′ (SEQ ID NO: 6).
100. The method of embodiment 99, wherein the polymerase is a T3 polymerase. 101. The method of any one of embodiments 90-100, wherein the sequence encoding the stem-loop is compatible with a Cpf1 protein.
102. The method of embodiment 101, wherein the sequence encoding the stem-loop comprises a sequence of 5′-AATTTCTACTGTTGTAGAT-3′ (SEQ ID NO: 8).
103. The method of any one of embodiments 90-102, wherein the sequence encoding the targeting sequence comprises a sequence that has at least 85%, at least 90%, at least 95%, at least 96%, at least 97%, at least 98% or at least 99% identity to a sequence that is located immediately 3′ of a protospacer adjacent motif (PAM) site in a sequence of a subject.
104. The method of any one of embodiments 90-102, wherein the sequence encoding the targeting sequence comprises a sequence that has 100% identity to a sequence that is located immediately 3′ of a PAM site in a sequence of a subject.
105. The method of embodiment 103 or 104, wherein the PAM site comprises a PAM site that is compatible with a Cpf1 system protein.
106. The method of any one of embodiments 103-105, wherein the PAM site comprises TTN, TCN or TGN.
107. The method of any one of embodiments 101-106, wherein the Cpf1 system protein comprises a Cpf1 system protein isolated or derived from Francisella tularensis, Acidaminococcus, Lachnospiraceae or Prevotella.
108. The method of any one of embodiments 103 or 104, wherein the sequence of the subject comprises a genomic DNA sequence.
109. The method of embodiment 103 or 104, wherein the sequence of the subject comprises a cDNA sequence.
110. The method of embodiment 103 or 104, wherein the subject is a eukaryote.
111. The method of embodiment 110, wherein the eukaryote is a human.
112. The method of embodiment 103-111, wherein the sequence of the subject comprises host DNA sequence.
113. A method of making a guide ribonucleic acid (gRNA) without at least one untemplated 3′ nucleotide, comprising:
   (a) providing a deoxyribonucleic acid (DNA) comprising, from 5′ to 3:
      (i) a sequence encoding a promoter,
      (ii) a sequence encoding a stem-loop,
      (iii) a sequence encoding a targeting sequence, and
      (iv) a sequence encoding a restriction site;
   (b) contacting the DNA of (a) with a polymerase to produce an RNA comprising, from 5′ to 3′, the sequence encoding the stem-loop (ii), the sequence encoding the targeting sequence (iii), the sequence encoding the restriction site (iv) and at least one additional untemplated 3′ nucleotide;
   (c) hybridizing the RNA of (b) to a single stranded DNA (ssDNA) comprising a sequence complementary to the sequence encoding the restriction site,
      wherein conditions are sufficient for the RNA of (b) and the ssDNA to form an RNA/DNA heteroduplex region; and
   (d) contacting the RNA/DNA heteroduplex region with a restriction enzyme;
      wherein conditions are sufficient for the restriction enzyme to hydrolyze a phosphodiester bond of the RNA in the RNA/DNA heteroduplex region,
      thereby generating a gRNA without at least one untemplated 3′ nucleotide.
114. A method of making a guide ribonucleic acid (RNA) without at least one untemplated 3′ nucleotide, comprising:
   (a) providing a deoxyribonucleic acid (DNA) comprising, from 5′ to 3:
      (i) a sequence encoding a promoter,
      (ii) a sequence encoding a stem-loop,
      (iii) a sequence encoding a targeting sequence, and
      (iv) a sequence encoding a restriction site;
      (v) a sequence encoding a primer binding sequence;
   (b) contacting the DNA of (a) with a polymerase to produce an RNA comprising, from 5′ to 3′, the sequence encoding the stem-loop (ii), the sequence encoding the targeting sequence (iii), the sequence encoding the restriction site (iv), the sequence encoding the primer binding sequence (v) and at least one additional untemplated 3′ nucleotide;
   (c) hybridizing the RNA of (b) to a single stranded DNA (ssDNA) comprising a sequence complementary to the sequence encoding the restriction site and the sequence encoding the primer binding sequence, wherein conditions are sufficient for the RNA of (b) and the ssDNA to form an RNA/DNA heteroduplex region; and
   (d) contacting the RNA/DNA heteroduplex region with a restriction enzyme;
      wherein conditions are sufficient for the restriction enzyme to hydrolyze at least one phosphodiester bond of the RNA in the RNA/DNA heteroduplex region, thereby generating a gRNA without at least one untemplated 3′ nucleotide.
115. The method of embodiment 113 or 114, wherein the restriction enzyme is a Type II restriction enzyme.
116. The method of embodiment 117, wherein the Type II restriction enzyme is a Type IIP restriction enzyme.
117. The method of embodiment 116, wherein the Type IIP restriction enzyme is selected from the group consisting of AvaII, AvrII, HaeIII, HinfI or TaqI.
118. The method of embodiment 115, wherein the restriction enzyme comprises SalI, HhaI, AluI, HindIII, EcoRI or MspI.
119. The method of any one of embodiments 113-118, wherein the DNA of (a) is a synthetic DNA. 120. The method of any one of embodiments 114-118, wherein the DNA of (a) is a PCR amplification product.
121. The method of embodiment 119 or 120, wherein the DNA of (a) is a plasmid,
   wherein the method further comprises contacting the plasmid with a restriction enzyme prior to the transcribing step of (b), and
   wherein conditions are sufficient to produce a linear plasmid DNA.
122. The method of any one of embodiments 113-121, wherein the sequence encoding the promoter is selected from the group consisting of a sequence encoding a T7 promoter, a sequence encoding an SP6 promoter or a sequence encoding a T3 promoter.
123. The method of embodiment 122, wherein the sequence encoding the T7 promoter comprises a sequence of 5′-TAATACGACTCACTATAGG-3′ (SEQ ID NO: 1).
124. The method of embodiment 123, wherein the polymerase is a T7 polymerase.
125. The method of embodiment 122, wherein the sequence encoding the SP6 promoter comprises a sequence of 5′-CATACGATTTAGGTGACACTATAG-3′ (SEQ ID NO: 5).
126. The method of embodiment 125, wherein the polymerase is an SP6 polymerase.
127. The method of embodiment 122, wherein the sequence encoding the T3 promoter comprises a sequence of 5′-AATTAACCCTCACTAAAG-3′ (SEQ ID NO: 6).
128. The method of embodiment 127, wherein the polymerase is a T3 polymerase.
129. The method of any one of embodiments 113-128, wherein the sequence encoding the stem-loop is compatible with a Cpf1 protein.
130. The method of embodiment 129, wherein the sequence encoding the stem-loop comprises a sequence of 5′-AATTTCTACTGTTGTAGAT-3′ (SEQ ID NO: 8).
131. The method of any one of embodiments 113-130, wherein the sequence encoding the targeting sequence comprises a sequence that has at least 85%, at least 90%, at least 95%, at least 96%, at least 97%, at least 98% or at least 99% identity to a sequence that is located immediately 3′ of a protospacer adjacent motif (PAM) site in a sequence of a subject.
132. The method of any one of embodiments 113-130, wherein the sequence encoding the targeting sequence comprises a sequence that has 100% identity to a sequence that is located immediately 3′ of a PAM site in a sequence of a subject.
133. The method of embodiment 131 or 132, wherein the PAM site comprises a PAM site that is compatible with a Cpf1 system protein.
134. The method of any one of embodiments 131-133, wherein the PAM site comprises TTN, TCN or TGN.
135. The method of any one of embodiments 130-133, wherein the Cpf1 system protein comprises a Cpf1 system protein isolated or derived from Francisella tularensis, Acidaminococcus, Lachnospiraceae or Prevotella.
136. The method of any one of embodiments 131 or 132, wherein the sequence of the subject comprises a genomic DNA sequence.
137. The method of embodiment 131 or 132, wherein the sequence of the subject comprises a cDNA sequence.
138. The method of embodiment 131 or 132, wherein the subject is a eukaryote.
139. The method of embodiment 138, wherein the eukaryote is a human.
140. The method of any one of embodiments 131-139, wherein the sequence of the subject comprises host DNA sequence.
141. A method of reducing the number of untemplated 3′ nucleotides in a guide ribonucleic acid (RNA), comprising:
   (a) providing a deoxyribonucleic acid (DNA) comprising, from 5′ to 3:
      (i) a sequence encoding a promoter,
      (ii) a sequence encoding a stem-loop, and
      (iii) a sequence encoding a targeting sequence;
   (b) contacting the DNA of (a) with a polymerase to produce a plurality of RNAs comprising, from 5′ to 3′, the sequence encoding the stem-loop, the sequence encoding the targeting sequence and at least one untemplated 3′ nucleotide; and
   (c) isolating at least one RNA from the plurality of RNAs;
      wherein the at least one isolated RNA is between 39 and 45 base pairs in length, thereby generating a gRNA with a reduced number of untemplated 3′ nucleotides. 142. The method of embodiment 141, wherein the at least one isolated RNA is 39 base pairs in length.
143. The method of embodiment 141 or 142, wherein the isolation step of (c) comprises:
   (i) running the plurality of RNAs and an RNA ladder on a gel,
   (ii) cutting out a region of the gel in the 39 to 48 bp size range, and
   (iii) extracting the RNA from the gel.
144. The method of claim 143, wherein the gel comprises a polyacrylamide gel.
145. The method of claim 144, wherein the isolating step of (c) comprises size exclusion chromatography.
146. The method of any one of embodiments 141-145, wherein the DNA of (a) is a synthetic DNA.
147. The method of any one of embodiments 141-145, wherein the DNA of (a) is a PCR amplification product.
148. The method of embodiment 146 or 147, wherein the DNA of (a) is a plasmid,
   wherein the method further comprises contacting the plasmid with a restriction enzyme prior to the transcribing step of (b), and
   wherein conditions are sufficient to produce a linear plasmid DNA.
149. The method of any one of embodiments 141-148, wherein the sequence encoding the promoter is selected from the group consisting of a sequence encoding a T7 promoter, a sequence encoding an SP6 promoter or a sequence encoding a T3 promoter.
150. The method of embodiment 149, wherein the sequence encoding the T7 promoter comprises a sequence of 5′-TAATACGACTCACTATAGG-3′ (SEQ ID NO: 1).
151. The method of embodiment 150, wherein the polymerase is a T7 polymerase.
152. The method of embodiment 149, wherein the sequence encoding the SP6 promoter comprises a sequence of 5′-CATACGATTTAGGTGACACTATAG-3′ (SEQ ID NO: 5).
153. The method of embodiment 152, wherein the polymerase is an SP6 polymerase.
154. The method of embodiment 149, wherein the sequence encoding the T3 promoter comprises a sequence of 5′-AATTAACCCTCACTAAAG-3′ (SEQ ID NO: 6).
155. The method of embodiment 154, wherein the polymerase is a T3 polymerase.
156. The method of any one of embodiments 141-155, wherein the sequence encoding the stem-loop is compatible with a Cpf1 system protein.
157. The method of any one of embodiments 141-156, wherein the sequence encoding the stem-loop comprises a sequence of 5′-AATTTCTACTGTTGTAGAT-3′ (SEQ ID NO: 8).
158. The method of any one of embodiments 141-157, wherein the sequence encoding the targeting sequence comprises a sequence that has at least 85%, at least 90%, at least 95%, at least 96%, at least 97%, at least 98% or at least 99% identity to a sequence that is located immediately 3′ of a protospacer adjacent motif (PAM) site in a sequence of a subject.
159. The method of any one of embodiments 141-157, wherein the sequence encoding the targeting sequence comprises a sequence that has 100% identity to a sequence that is located immediately 3′ of a PAM site in a sequence of a subject.
160. The method of embodiment 158 or 159, wherein the PAM site comprises a PAM site that is compatible with a Cpf1 system protein.
161. The method of any one of embodiments 158-160, wherein the PAM site comprises TTN, TCN or TGN.
162. The method of any one of embodiments 157-161, wherein the Cpf1 system protein comprises a Cpf1 system protein isolated or derived from Francisella tularensis, Acidaminococcus, Lachnospiraceae or Prevotella.
163. The method of any one of embodiments 158 or 159, wherein the sequence of the subject comprises a genomic DNA sequence.
164. The method of embodiment 158 or 159, wherein the sequence of the subject comprises a cDNA sequence.
165. The method of embodiment 158 or 159, wherein the subject is a eukaryote.
166. The method of embodiment 165, wherein the eukaryote is a human.
167. The method of embodiment 158-166, wherein the sequence of the subject comprises host DNA sequence.

### EXAMPLES

### Example 1: Ligation-Free Library Preparation

A short PCR product was used to produce a sequenceable library using the following protocol:

### Protocol Overview

### Part 1—Blunt Ending

The PCR product was blunt ended using T4 DNA polymerase. The ends of the DNA need to be blunt for T4 DNA polymerases such as Klenow to efficiently add dNTPs or ddNTPs.

Following blunt ending, QiaQuick cleanup was used to remove remaining nucleotides. Optionally, recombinant shrimp alkaline phosphatase (rSAP) enzymatic cleanup, a bead based cleanup or other column can be used to remove nucleotides at this point.

### Part 2—Blocking

3′ end blocking was carried out using ddNTPs and Klenow. Sequencing suggests that this step, and therefore perhaps also the blunt ending step, may not be necessary. Most sequences after sequencing were unblocked, indicating that the blocking step may not be necessary. If the blunt ending is needed, but not the blocking, since the enzyme is heat denatured, it may be possible to skip the post-blunting purification prior to this step.

Following 3′ end blocking, QiaQuick cleanup was used to remove remaining nucleotides. Optionally, rSAP enzymatic cleanup, a bead based cleanup or other column can be used to remove nucleotides at this point.

Note: The initial sequencing results indicates that this step (and therefore even the blunt end step) may not be necessary.

Part 3—Adapter 1 addition

A single-sided PCR (i.e., with only one primer) that allows the adapter+primer to anneal and extend the length of the DNA was carried out. Initially, this step was carried out with Taq polymerase. However, high fidelity polymerases may be used going forward. Optionally, isothermal amplification, for example using Phi29 DNA polymerase, can be used.

Following single-sided PCR, a MinElute PCR purification kit was used to isolate the single-sided PCR product. Optionally, rSAP enzymatic cleanup, a bead based cleanup or other column can be used to isolate the PCR product at this point.

### Part 4—Tailing

The single-sided PCR product was polyadenylated (A-tailed) using a Terminal Transferase. Optionally, a polyG tail can be used, and is less variable with respect to the concentration of the DNA input.

Following polyadenylation, a MinElute PCR purification kit was used to isolate the A-tailed DNA. Optionally, rSAP enzymatic cleanup, a bead based cleanup or other column can be used to isolate the tailed DNA at this point.

### Part 5—Adapter 2 Addition

The tailed PCR product was then used as a template in a second single-sided PCR (i.e., only one primer) that allowed the second adapter+primer to anneal to the Poly-A tail and extend the full length of the molecule, thus including the adapter on the other side of the PCR product. Initially, this step was carried out with Taq polymerase. However, high fidelity polymerases may be used going forward. Optionally, isothermal amplification, for example using Phi29 DNA polymerase, can be used.

Following the second single-sided PCR reaction, a MinElute PCR purification kit was used to isolate the A-tailed DNA. Optionally, a bead based cleanup or other column can be used to isolate the PCR product at this point.

The PCR product was then checked by qPCR. Successful qPCR amplification indicated that a sequenceable library had been made.

### Part 6—Indexing PCR

A standard indexing PCR reaction was used to add barcodes to adapters, followed by Kapa bead purification

### Part 7—Sequencing

Standard high throughput sequencing methods were used to sequence the library. Optionally a one tube reaction (i.e., all enzymatic clean ups until the indexing, combining steps potentially Poly-G tailing then heat inactivating and adding Adapter 2) can be used. An additional variation of the protocol is the adapter 1 addition, followed poly-g tailing, then adapter 2 addition and finally indexing PCR (no blunt or blocking).

### Detailed Protocol

The following samples were processed according to the protocol set forth below.
(1) Negative control (water, called “Negative”), the 3′ end was not blocked
(2) 64 bp DNA digested into 2 parts by MseI to test blocking efficiency (called “Positive”), the 3′ end was not blocked
(3) 64 bp DNA digested into 2 parts by MseI to test blocking efficiency (called “Test”), the 3′ end was blocked.
   Unless otherwise indicated, sample PCR products, rSAP products/DNA, Klenow products were treated the same during processing.

### Detailed Protocol

### Part 1—Blunt Ending

The blunt ending was carried out using the conditions shown in Table 3 below:

TABLE 3
Blunt ending

| | Per Sample | Initial | | final | |
|---|---|---|---|---|---|
| Reagent | (ul) | concentration | | concentration | |
| T4 DNA | 2.0 | 3 | U/ul | 0.12 | U/ul |
| Polymerase | | | | | |
| Cutsmart Buffer | 0.40 | 10× | | 1× | |
| dNTPs | 1.60 | 10 | mM each | 48.5 | uM each |
| PCR product | 29.0 | 26.8 | ng/ul | 723 | ng total |

| | | | |
|---|---|---|---|
| Water | 0.00 | — | — |
| Sum | 33 | | |

1 Unit (U) T4 DNA polymerase per μg DNA was used. PCR product was from the NL01 SNP PCR, and was MseI digested. The reaction was incubated at 12° C. for 15 minutes, and then at 75° C. for 20 minutes. A Qiaquick PCR purification kit was used to remove nucleotides from 33 μL to 65 μL of the reaction mixture.

### Part 2—Blocking

The blunt ended PCR product was blocked using the conditions shown in Tables 4-6 below:

TABLE 4
Sample 1: Klenow Negative Control (with water) - No tail

| | Per Sample | Initial | | final | |
|---|---|---|---|---|---|
| Reagent | (ul) | concentration | | concentration | |
| Klenow (exo-) | 3 | 5 | U/ul | 0.3 | U/ul |
| Cutsmart Buffer | 5 | 10× | | 1× | |
| dNTPs | 2.5 | 10 | mM each | 500 | uM each |

| | | | |
|---|---|---|---|
| Water (no DNA) | 30 | — | — |
| Water | 9.5 | — | — |
| Sum | 50 | | |

TABLE 5
Sample 2: Klenow Positive Control (with DNA + dNTPS) - Tail

| | Per Sample | Initial | | final | |
|---|---|---|---|---|---|
| Reagent | (ul) | concentration | | concentration | |
| Klenow (exo-) | 3 | 5 | U/ul | 0.3 | U/ul |
| Cutsmart Buffer | 5 | 10× | | 1× | |
| dNTPs | 2.5 | 10 | mM each | 500 | uM each |
| rSAP product | 30 | 13 | ng/ul | 5.2 | ng/ul |

| | | | |
|---|---|---|---|
| Water | 9.5 | — | — |
| Sum | 50 | | |

TABLE 6
Sample 3: Klenow Test (with DNA + ddNTPs) - Testing

| | Per Sample | Initial | | final | |
|---|---|---|---|---|---|
| Reagent | (ul) | concentration | | concentration | |
| Klenow (exo-) | 3 | 5 | U/ul | 0.3 | U/ul |
| Cutsmart Buffer | 5 | 10× | | 1× | |
| ddNTPs | 0.5 | 2.5 | mM each | 500 | uM each |
| rSAP product | 30 | 13 | ng/ul | 5.2 | ng/ul |

| | | | |
|---|---|---|---|
| Water | 11.5 | — | — |
| Sum | 50 | | |

All samples were incubated for 40 minutes at 37° C., and then for 75° C. for 20 minutes. Excess nucleotides were then removed using the Qiaquick Nucleotide removal kit, and eluted into 50 μL elution buffer (EB).

### Part 3—Adapter 1

Single-sided Adapter 1 PCR was carried out using the following reaction conditions:

TABLE 7
Adapter 1 PCR Reaction Mixture

| | Per Sample | Initial | final |
|---|---|---|---|
| Reagent | (ul) | concentration | concentration |
| Taq 2X MM | 110.5 | 2× | 1× |
| NL01_Rev+Adapter | 4.4 | 10 uM | 0.2 uM |
| Klenow product | 20 | | |
| Water | 86.08 | — | — |
| Sum | 221 | | |

The primer was designed to target a phenotypic SNP present in the PCR product, and also had an NEBNext Adapter attached.

TABLE 8
Adapter 1 PCR Reaction Conditions

| Run for: | |
|---|---|
| 95° C. for 3 min | |
| 95° C. for 30 sec | 45 cycles |
| 68° C. for 60 sec | |
| 68° C. for 5 min | |
| 12° C. hold | |

Other, higher fidelity polymerases, for example the Qiagen high fidelity polymerase master mix (MM), may also be suitable. It may also be possible to vary the number of cycles (i.e., use more than 45 or less than 45 cycles). Following single-sided PCR, the MinElute PCR purification kit was used to purify the PCR product. This removed unincorporated nucleotides and small un-extended fragments. 221 μL PCR product were eluted into 60 μL EB.

### Part 4—A-tailing

PCR products were polyadenylated using the following reaction conditions:

TABLE 9
Polyadenylation Reaction

| | Per Sample | Initial | | final | |
|---|---|---|---|---|---|
| Reagent | (ul) | concentration | | concentration | |
| Tdt buffer | 7.5 | 10× | | 1× | |
| CoCl2 Solution | 7.5 | 2.5 | mM | 0.25 | mM |
| dATP | 2.7 | 1 | mM | 2,737 | |
| Terminal transferase | 0.8 | 20 | U/ul | 0.2 | U/ul |

| | | | |
|---|---|---|---|
| DNA | 50 | 1.37 | pmol |
| Water | 6.5 | — | — |
| Sum | 75 | | |

For dATP, 1:1000 pmol ends to pmol dNTPs was used. 0.2 U/μL Terminal Transferase for up to 5 pmol were used. 52 ng of DNA were used for the Test and Negative samples, 101 ng DNA was used for the Positive sample. Reactions were incubated at 37° C. for 30 minutes, and then at 70° C. for 10 minutes. A MinElute Reaction cleanup kit was used to purify poly Adenylated PCR products. 75 μL of polyadenylated PCR product were eluted into 40 μL of EB.

### Part 5—Adapter 2 Addition

The second adapter was added using the following PCR conditions:

TABLE 10
Adapter 2 PCR Reaction Mixture

| | Per Sample | Initial | final |
|---|---|---|---|
| Reagent | (ul) | concentration | concentration |
| Taq 2X MM | 100 | 2× | 1× |
| P7_PolyT_Adapter | 4.0 | 10 uM | 0.2 uM |
| DNA | 35 | | |
| Water | 61 | — | — |
| Sum | 200 | | |

The second primer was designed to have a polyT sequence with an NEBNext adapter sequence attached.

TABLE 11
Adapter 2 PCR Reaction Conditions

| Run for: | |
|---|---|
| 95° C. for 3 min | |
| 95° C. for 30 sec | 45 cycles |
| 60° C. for 60 sec | |
| 68° C. for 60 sec | |
| 12° C. hold | |

Other, higher fidelity polymerases, for example the Qiagen high fidelity polymerase master mix (MM), may also be suitable. It may also be possible to vary the number of cycles (i.e., use more than 45 or less than 45 cycles). A MinElute Reaction cleanup kit was used to purify polyAdenylated PCR products. 200 μL PCR product were eluted into 30 μL of EB. The PCR product was checked by qPCR amplification. Successful amplification indicated a sequenceable library had been made.

Part 6—Indexing PCR (iPCR1)

Indexing PCR to add barcodes to the library was carried out as follows:

TABLE 12
Indexing PCR Reaction Mixture

| | Per Sample | | Initial | | final | |
|---|---|---|---|---|---|---|
| Reagent | (ul) | x3 | concentration | | concentration | |
| Kapa HiFi | 5.00 | 15 | 5× | | 1× | |
| Buffer | | | | | | |
| Kapa dNTP | 0.75 | 2.25 | 10 | mM each | 0.3 | mM each |
| mix | | | | | | |
| Kapa HiFi | 0.50 | 1.5 | 1 | U/ul | 0.5 | U total |
| Polym | | | | | | |
| Fwd (i5) | 0.75 | 2.3 | 10 | uM | 0.3 | uM |
| Rev (i7) | 0.75 | 2.3 | 10 | uM | 0.3 | uM |

| | | | | |
|---|---|---|---|---|
| Water | 17.25 | 51.75 | — | — |
| Sum | 25 | 25 | | |

NEBNext indexes that amplify only NEBNext adapters were used on the indexing primers. 5 μL DNA (post Adapter 2 addition) was added.

TABLE 13
Indexing PCR Reaction Conditions

| Run for: | |
|---|---|
| 95° C. for 3 min | |
| 98° C. for 20 sec | 6 Cycles |
| 60° C. for 15 sec | |
| 72° C. for 20 sec | |
| 72° C. for 3 min | |
| 12° C. hold | |

*The number of cycles was calculated based off of qPCR plateau values.

Following indexing PCR, Kapa bead purification was used to purify the PCR product. 25 μL of PCR product was eluted into 25 μL EB.

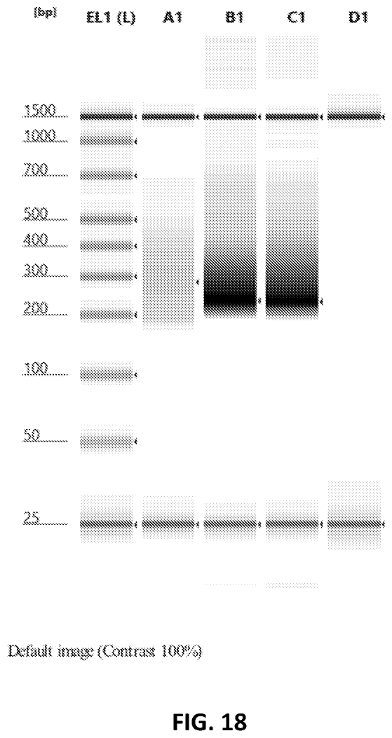

The Positive, Negative and Test sample libraries created with this protocol, as well as an A-tail negative control, were quantified using the Agilent High Sensitivity D1000 ScreenTape System following indexing PCR and purification, and the results are shown in FIGS. 18-24 below. See Table 14 below for sample/well identity and concentration, and Tables 15-23 for quantification corresponding to FIGS. 19-23.

TABLE 14
Sample Information

| | Concentration | | | |
|---|---|---|---|---|
| Well | (pg/μL) | Sample Description | Alert | Observations |
| EL1 | 2350 | Electronic Ladder | | Ladder |
| A1 | 124 | iPCR1-Pur-Neg | | |
| B1 | 7140 | iPCR1-Pur-Test | | |
| C1 | 6380 | iPCR1-Pur-Pos | | |
| D1 | | PCR10-Atail-Neg | | |

Neg = Negative (sample 1),
Test = Test (sample 3),
Pos = Positive (sample 2),
Atail-Neg = Atailing negative control.

TABLE 15
Electronic Ladder Peak Table

| | Calibrated | Assigned | Peak | % | Peak | |
|---|---|---|---|---|---|---|
| Size | Conc. | Conc. | Molarity | Integrated | Com- | |
| [bp] | [pg/μL] | [pg/μL] | [pmol/l] | Area | ment | Observations |
| 25 | 340 | — | 20900 | — | | Lower Marker |
| 50 | 265 | — | 8160 | 11.28 | | |
| 100 | 278 | — | 4270 | 11.82 | | |
| 200 | 290 | — | 2230 | 12.32 | | |
| 300 | 304 | — | 1560 | 12.95 | | |
| 400 | 306 | — | 1180 | 13.00 | | |
| 500 | 312 | — | 961 | 13.29 | | |
| 700 | 286 | — | 629 | 12.19 | | |
| 1000 | 309 | — | 476 | 13.15 | | |
| 1500 | 250 | 250 | 256 | — | | Upper Marker |

TABLE 16
iPCR1-Pur-Neg Peak Table

| | Calibrated | Assigned | Peak | % | Peak | |
|---|---|---|---|---|---|---|
| Size | Conc. | Conc. | Molarity | Integrated | Com- | |
| [bp] | [pg/μL] | [pg/μL] | [pmol/l] | Area | ment | Observations |
| 25 | 425 | — | 26200 | — | | Lower Marker |
| 286 | 124 | — | 665 | 100.00 | | |
| 1500 | 250 | 250 | 256 | — | | Upper Marker |

TABLE 17
iPCR1-Pur-Neg Region Table

| | | | | Region | | | |
|---|---|---|---|---|---|---|---|
| From | To | Average | Conc. | Molarity | % of | Region | |
| [bp] | [bp] | Size [bp] | [pg/μL] | [pmol/l] | Total | Comment | Color |
| 100 | 1000 | 331 | 1840 | 9560 | 96.75 | | Dark |
| 265 | 1000 | 387 | 1230 | 5240 | 64.55 | | Light |

TABLE 18
iPCR1-Pur-Test Peak Table

| | Calibrated | Assigned | Peak | % | Peak | |
|---|---|---|---|---|---|---|
| Size | Conc. | Conc. | Molarity | Integrated | Com- | |
| [bp] | [pg/μL] | [pg/μL] | [pmol/l] | Area | ment | Observations |
| 25 | 383 | — | 23600 | — | | Lower Marker |
| 237 | 7140 | — | 46400 | 100.00 | | |
| 1500 | 250 | 250 | 256 | — | | Upper Marker |

TABLE 19
iPCR1-Pur-Test Region Table

| | | | | Region | | | |
|---|---|---|---|---|---|---|---|
| From | To | Average | Conc. | Molarity | % of | Region | |
| [bp] | [bp] | Size [bp] | [pg/μL] | [pmol/l] | Total | Comment | Color |
| 100 | 1000 | 309 | 10400 | 57000 | 97.05 | | Dark |
| 265 | 1000 | 373 | 5540 | 25100 | 51.50 | | Light |

TABLE 20
iPCR1-Pur-Pos Peak Table

| | Calibrated | Assigned | Peak | % | Peak | |
|---|---|---|---|---|---|---|
| Size | Conc. | Conc. | Molarity | Integrated | Com- | |
| [bp] | [pg/μL] | [pg/μL] | [pmol/l] | Area | ment | Observations |
| 25 | 404 | — | 24900 | — | | Lower Marker |
| 235 | 6380 | — | 41900 | 100.00 | | |
| 1500 | 250 | 250 | 256 | — | | Upper Marker |

TABLE 21
iPCR1-Pur-Pos Region Table

| | | | | Region | | | |
|---|---|---|---|---|---|---|---|
| From | To | Average | Conc. | Molarity | % of | Region | |
| [bp] | [bp] | Size [bp] | [pg/μL] | [pmol/l] | Total | Comment | Color |
| 100 | 1000 | 305 | 9660 | 53100 | 97.32 | | Dark |
| 265 | 1000 | 367 | 5100 | 23200 | 51.31 | | Light |

TABLE 22
PCR10-Atail-Neg Peak Table

| | Calibrated | Assigned | Peak | % | Peak | |
|---|---|---|---|---|---|---|
| Size | Conc. | Conc. | Molarity | Integrated | Com- | |
| [bp] | [pg/μL] | [pg/μL] | [pmol/l] | Area | ment | Observations |
| 25 | 376 | — | 23200 | — | | Lower Marker |
| 1500 | 250 | 250 | 256 | — | | Upper Marker |

TABLE 23
PCR10- Atail-Neg Region Table

| | | | | Region | | | |
|---|---|---|---|---|---|---|---|
| From | To | Average | Conc. | Molarity | % of | Region | |
| [bp] | [bp] | Size [bp] | [pg/μL] | [pmol/l] | Total | Comment | Color |
| 100 | 1000 | 440 | 5.59 | 45.5 | 5.13 | | Dark |
| 265 | 1000 | 642 | 3.13 | 12.6 | 2.88 | | Light |

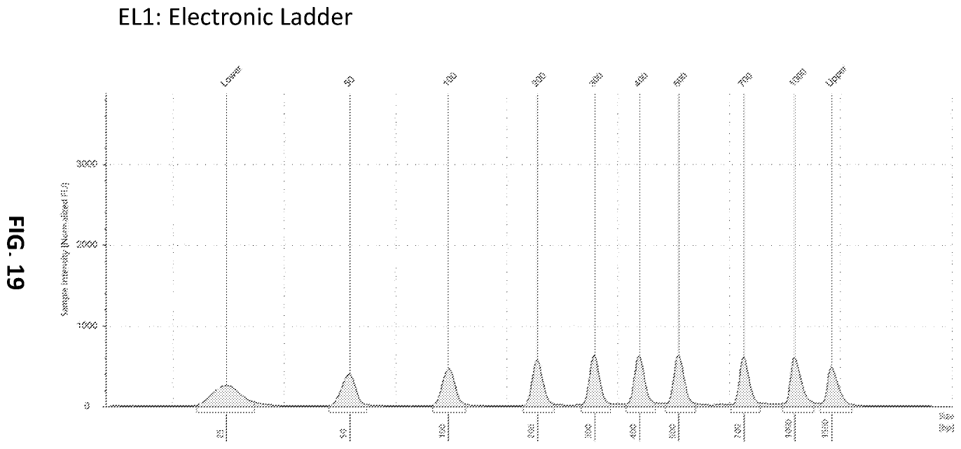

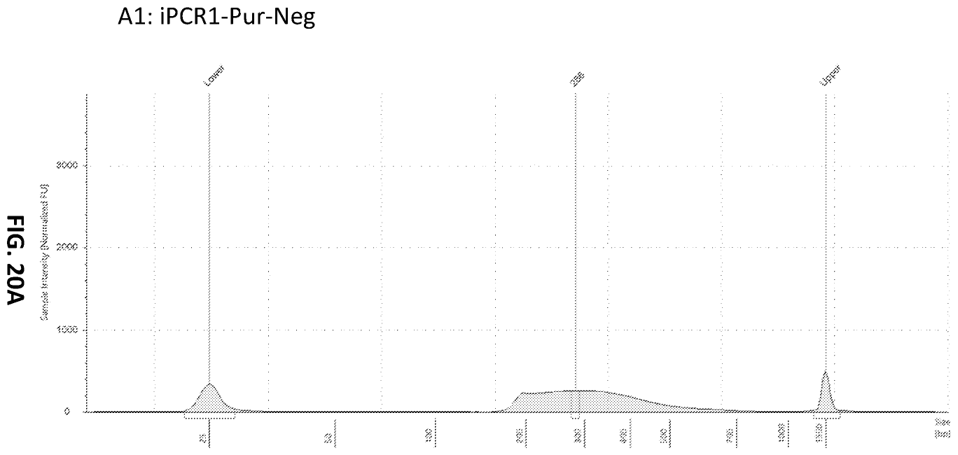

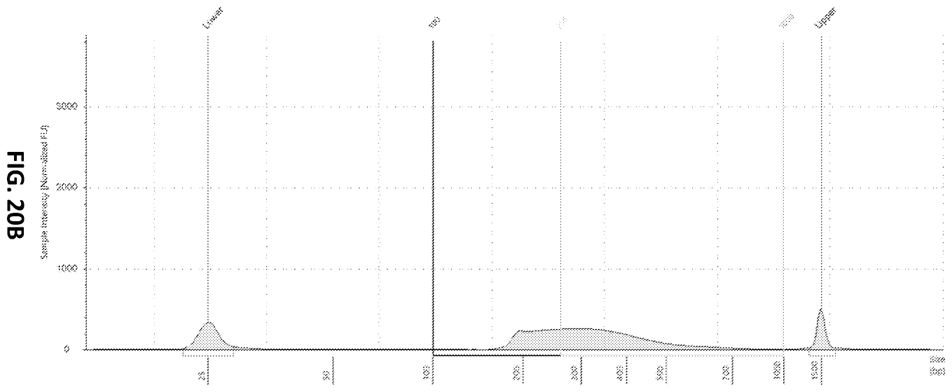

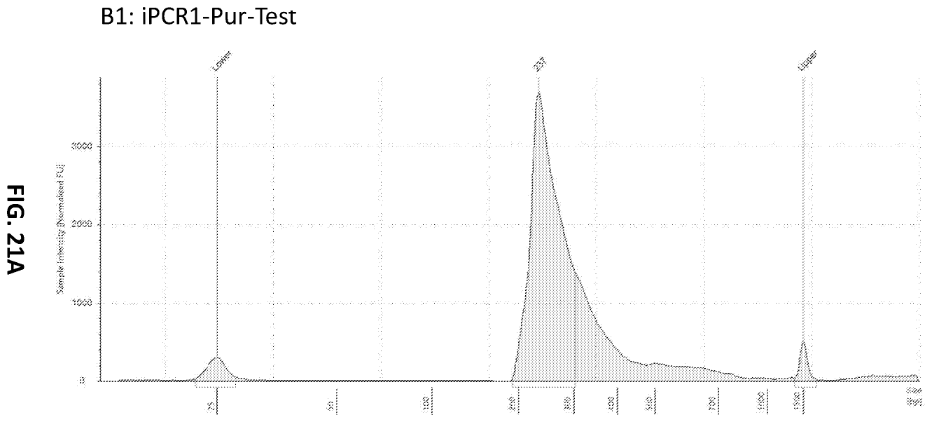

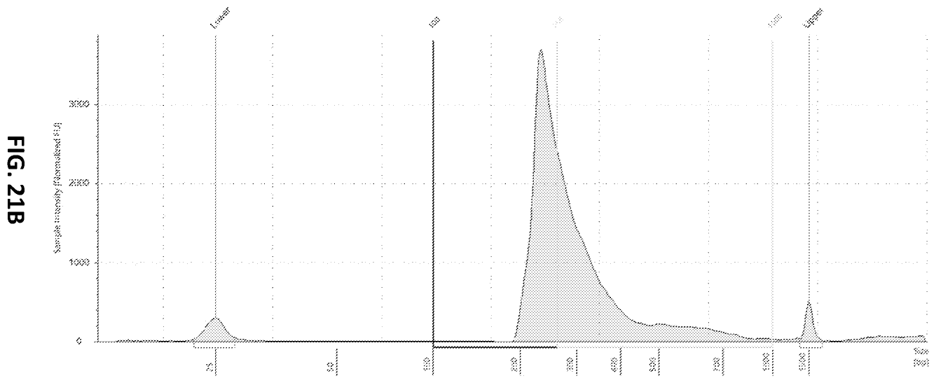

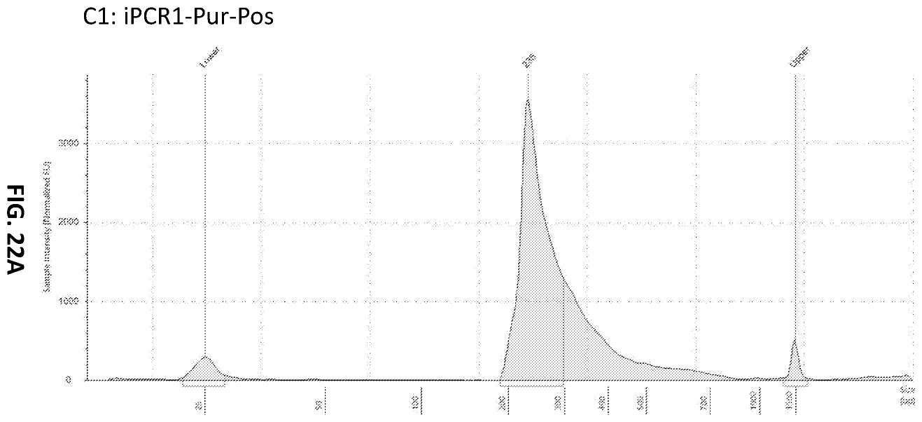

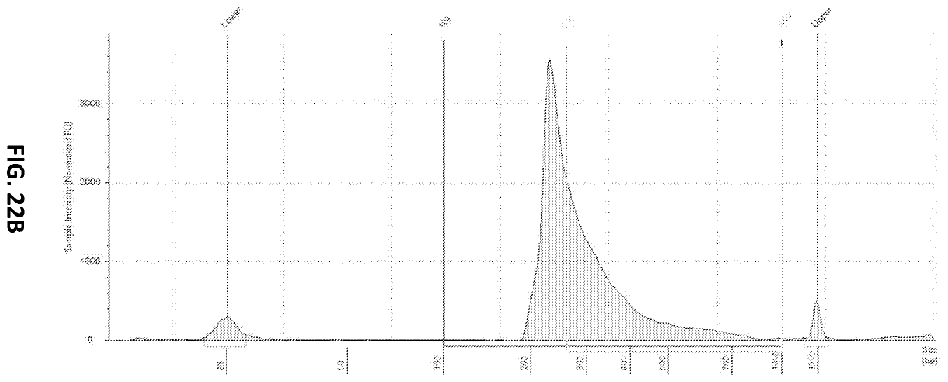

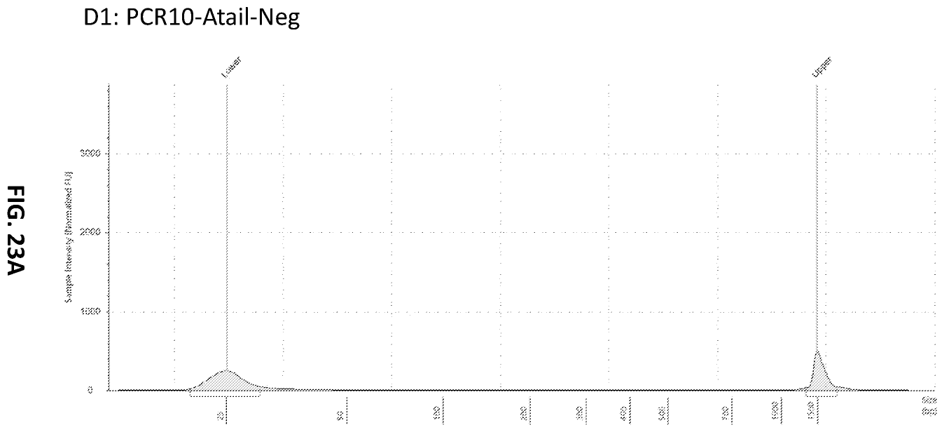

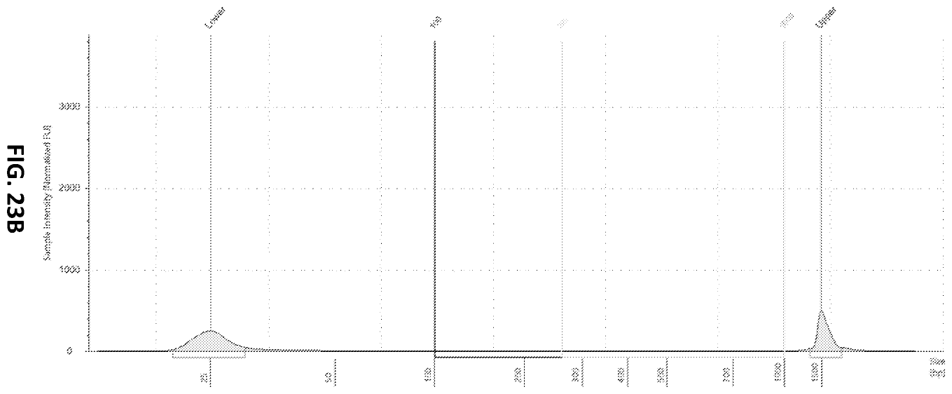

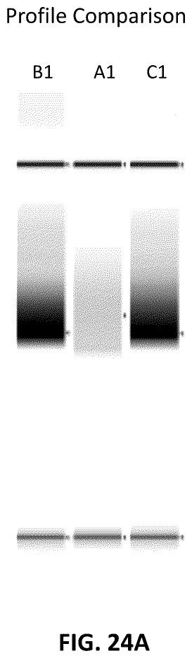

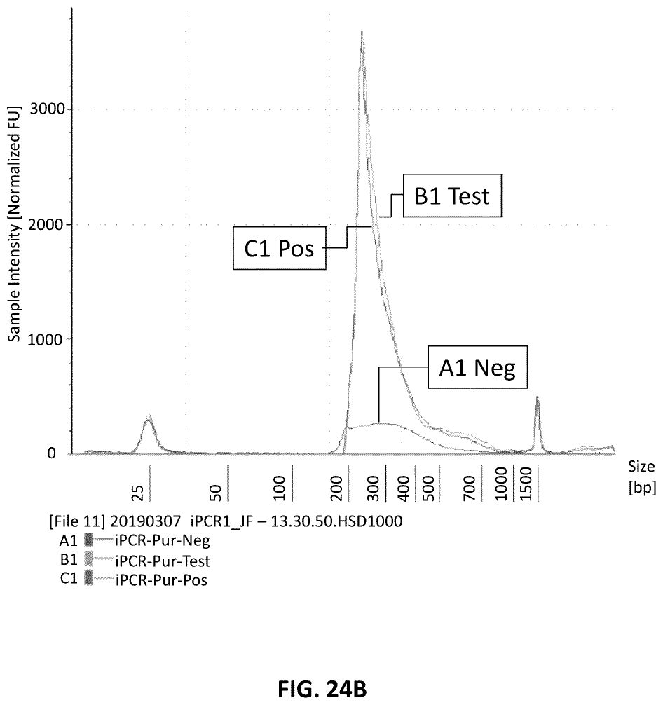

FIG. 18 shows a picture of the gel. FIG. 19 shows the ladder, while FIG. 20A-20B, FIG. 21A-21B, FIG. 22A-22B and FIG. 23 show High Sensitivity D1000 ScreenTape results for the Negative, Test, Positive and Atail negative control samples, respectively. FIG. 24A and FIG. 24B C show a comparison of the Positive, Negative and Test libraries.

Once purified, the Positive and Test libraries were high throughput sequenced.

### Example 2: Library Analysis

FastQC analysis was done on the trimmed, complexity and quality filtered data from Run 2 of both samples (Positive and Test). Analysis of the high throughput dataset was carried out using Samtools and FastQC, and the data summarized using MultiQC. Table 24 shows an overview of the general statistics from the two libraries.

TABLE 24
General Statistics

| | Reads | % | Average | Total |
|---|---|---|---|---|
| Sample | Mapped | Duplicate | % | Sequences |
| Name | (millions) | Reads | GC | (Millions) |
| Positive | 1 | 95% | 49% | 1 |
| Test | 0.3 | 95.40% | 49% | 0.3 |

Table 25 shows the output from the Samtools flagstat function, which does a full pass through the input file and calculates and prints the statistics. Results are in Millions of reads.

TABLE 25
Samtools Flagstat Output

| Parameter | Test | Positive |
|---|---|---|
| Total Reads | 0.27M | 0.96M |
| Total Passed QC | 0.27M | 0.96M |
| Mapped | 0.27M | 0.96M |
| Duplicates | 0.0M | 0.0M |
| Paired in Sequencing | 0.0M | 0.0M |
| Properly Paired | 0.0M | 0.0M |
| Self mate mapped | 0.0M | 0.0M |
| Singletons | 0.0M | 0.0M |
| Mapped to different | 0.0M | 0.0M |
| chromosome | | |
| Diff chr (MapQ >= 5) | 0.0M | 0.0M |

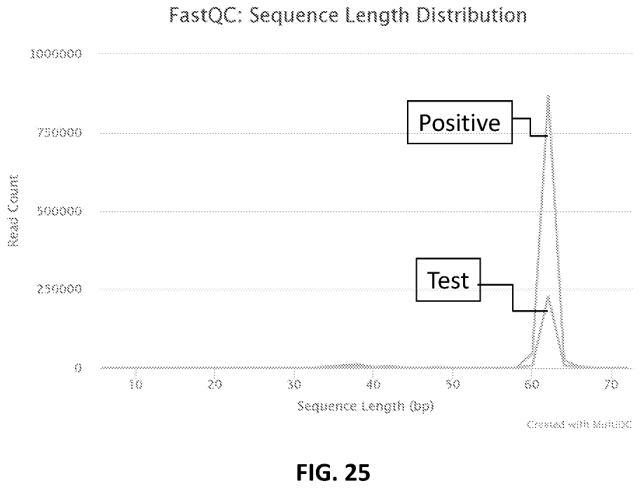

The sequencing showed that mainly the full-length 64 bp product was successfully sequenced, rather than the blocked, shorter fragments (this can be seen from the fragment size distribution shown in FIG. 25). Hence, it may be possible to omit the blocking and blunting steps.

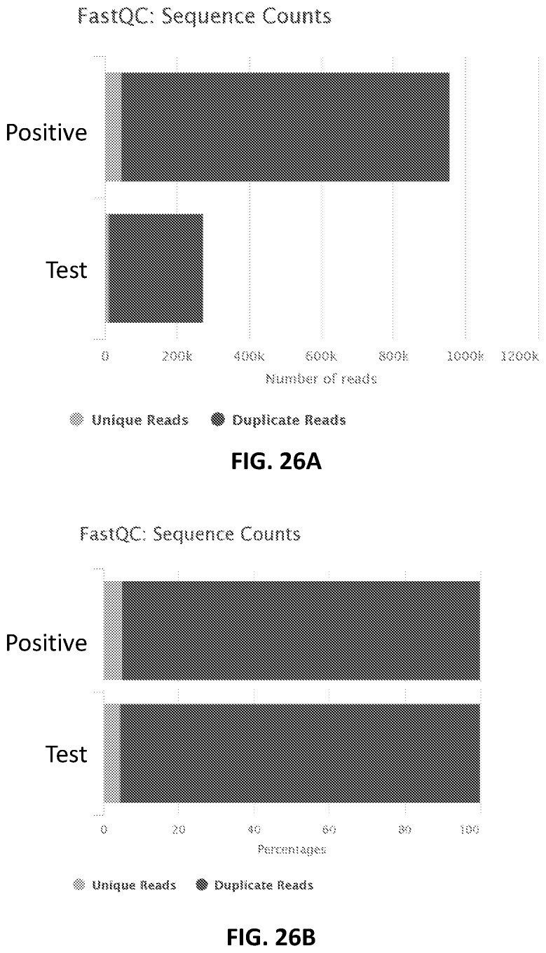

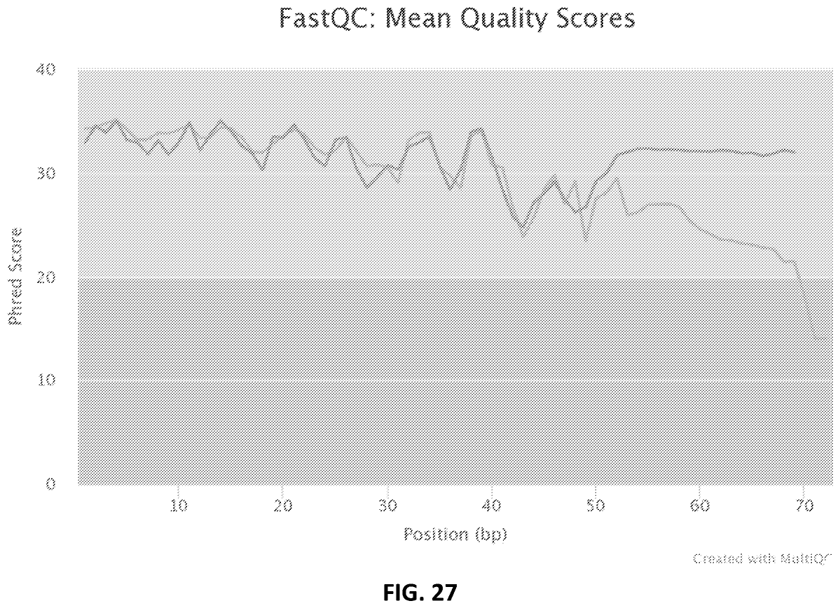

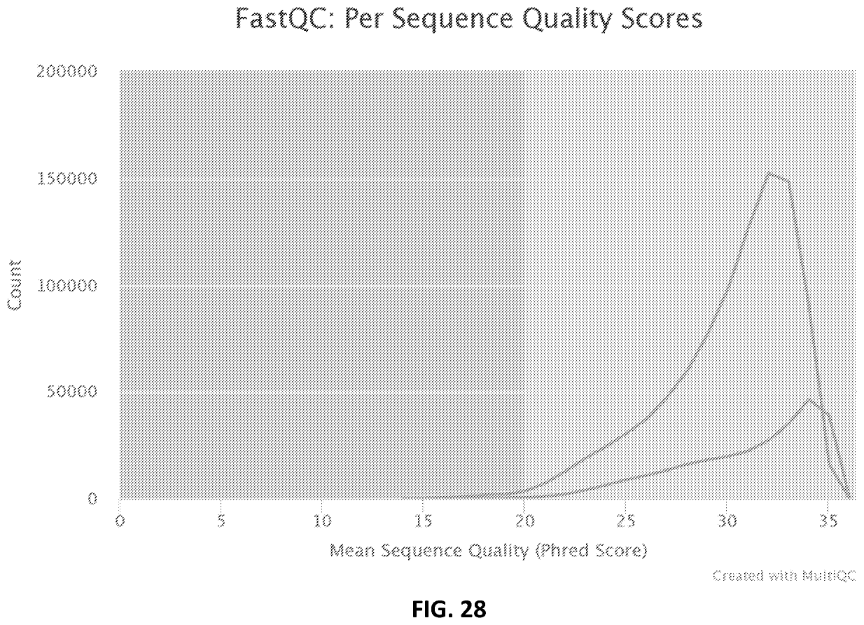

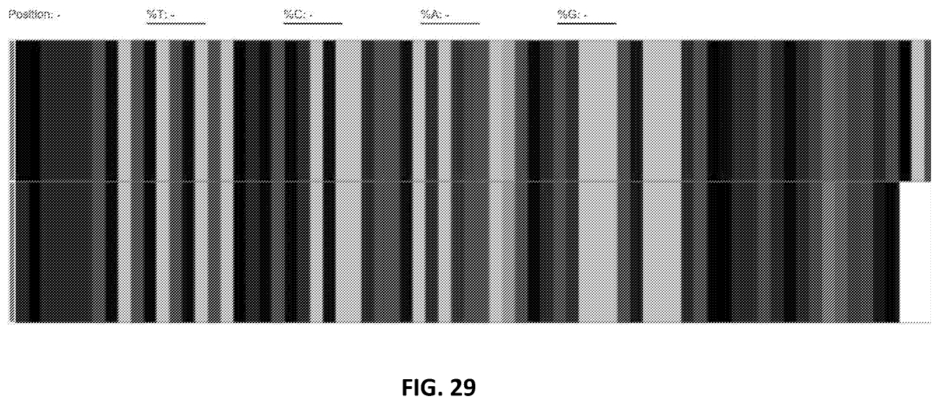

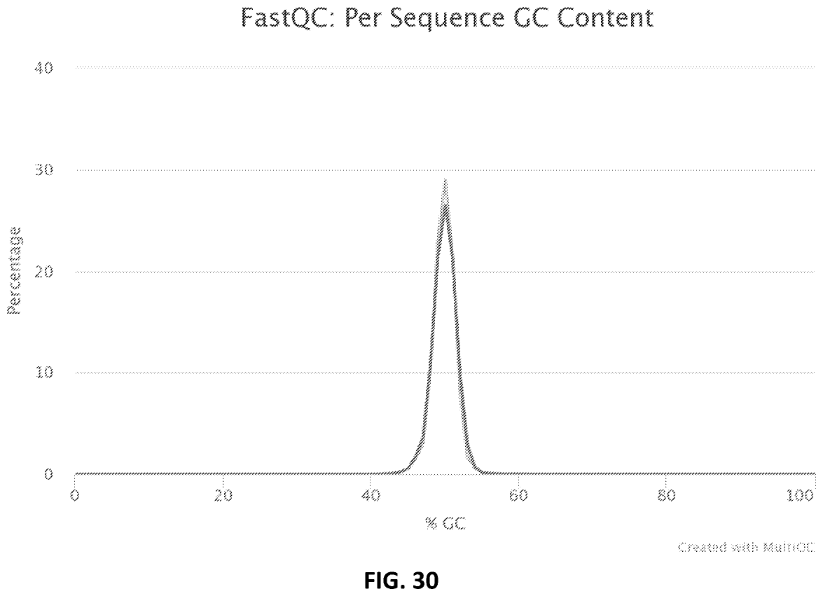

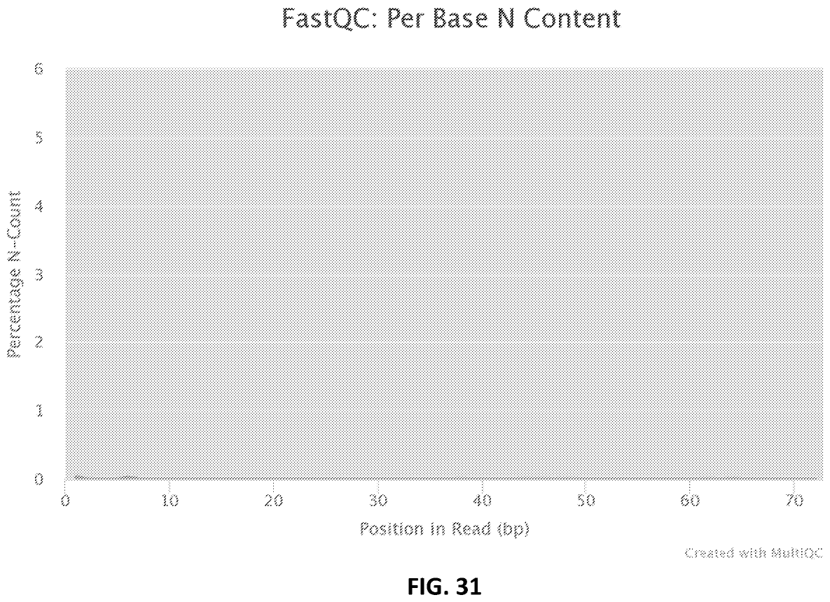

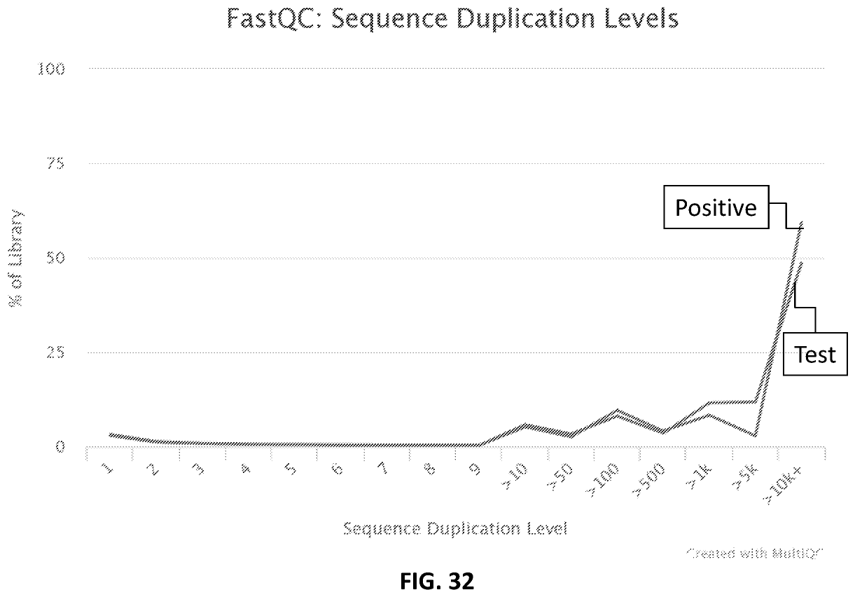

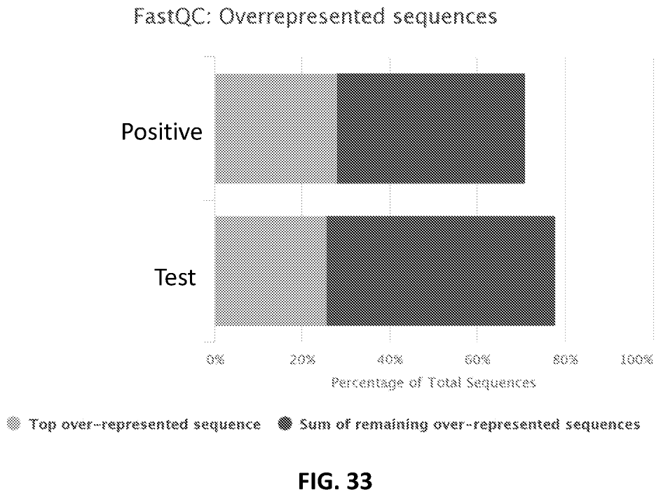

The samples went on two runs since the first did not produce enough data. In the first run, the Positive sample produced 74 reads. In the second run, the Positive sample produced 1,095,378 reads. 957,262 of these reads (87%) mapped sufficiently to the expected sequence. In the first run, the Test sample produced 385 reads. In the second run, the Test sample produced 289,368 reads. 272,245 of these reads (94%) mapped sufficiently to the expected sequence. No statistics are provided for the Run 1, since the read count was so low that the results are likely to just be sporadic. Statistics for Run 2 are presented in FIG. 25, FIG. 26A, FIG. 26B, FIG. 27, FIG. 28, FIG. 29, FIG. 30, FIG. 31, FIG. 32 and FIG. 33.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-58. (canceled)

59. A method of preparing a library of nucleic acids, comprising:
a. providing a sample of nucleic acids comprising at least one sequence of interest; b. contacting the sample of nucleic acids with a terminal transferase and NTPs under conditions sufficient to transfer NTPs to the 3′ end of the nucleic acids thereby generating a plurality of nucleic acids comprising 3′ tails;
c. contacting the plurality of nucleic acids comprising 3′ tails with a plurality of first adapters and a reverse transcriptase under conditions sufficient for first strand complementary DNA (cDNA) synthesis to occur, thereby generating a plurality of cDNAs; and
d. contacting the plurality of cDNAs with a second adapter under conditions sufficient to allow generation of double stranded DNA from the plurality of cDNAs to generate a plurality of double stranded DNAs,
thereby preparing a library of nucleic acids with adapters at the 5′ and 3′ ends.

60. The method of claim 60, wherein the plurality of first adapters comprise a sequence complementary to the 3′ tails and a first UMI sequence.

61. The method of claim 60, wherein the plurality of second adapters comprise a second UMI and a polyG sequence.

62. The method of claim 59, wherein the nucleic acids comprise ribonucleic acids (RNAs).

63. The method of claim 59, wherein the reverse transcriptase comprises Moloney Murine Leukemia Virus (MMLV) reverse transcriptase.

64. The method of claim 59, wherein step (d) comprises adding a polymerase.

65. The method of claim 64, wherein step (d) comprises PCR amplification of the plurality of double stranded DNAs.

66. The methods of claim 60, wherein the first unique molecular identifier sequence (UMI) comprises 2, 3, 4, 5, 6, 7, 8, 9, 10, 11 or 12 nucleotides.

67. The method of claim 65, wherein the first UMI is a random sequence.

68. The method of claim 59, wherein the first adapter comprises a sequence of a first sequencing adapter.

69. The method of claim 61, wherein the second UMI comprises 2, 3, 4, 5, 6, 7, 8, 9, 10, 11 or 12 nucleotides.

70. The method of claim 69, wherein the second UMI is a random sequence.

71. The method of any one of claims 59-70, wherein the second adapter comprises a sequence of a second sequencing adapter.

72. The method of claim 59, wherein the sequence of interest comprises a single nucleotide polymorphism (SNP), a miniSTR (mini short tandem repeat), a mitochondrial marker, a Y chromosome marker, or a disease trait marker.

73. The method of claim 72, wherein the disease trait marker comprises a marker for pathogenicity, virulence, resistance or strain identification.

74. The method of claim 59, wherein the sample is degraded.

75. The method of claim 59, wherein the sample is a forensics sample.

76. The method of claim 59, wherein the at least one sequence of interest comprises at least 1, 2, 3, 4, 5, 6, 7, 8, 9, 10, 15, 20, 25, 30, 40, 50, 60, 70, 80, 90, 100, 500, 1000, 10,000, 50,000, 100,000 or 200,000 unique sequences of interest.

77. The method of claim 59, wherein the sample of nucleic acids comprises ribonucleic acids (RNAs).

78. The method of claim 59, comprising sequencing the library of nucleic acids.

79. The method of claim 78, wherein the sequencing comprises high-throughput sequencing.

80. The methods of claim 59, comprising:
a. providing a plurality of guide nucleic acid (gNA)-CRISPR/Cas system protein complexes, wherein the gNAs are configured to hybridize to at least one sequence targeted for depletion;
b. mixing the library of nucleic acids with the plurality of gNA-CRISPR/Cas system protein complexes,
wherein at least a portion of the gNA-CRISPR/Cas system protein complexes hybridize to the at least one sequence targeted for depletion; and
c. incubating the mixture to cleave the at least one sequence targeted for depletion.

81. The method of claim 80, comprising PCR amplifying the library of nucleic acids following step (c).

82. The method of claim 80, wherein the CRISPR/Cas system protein comprises Cpf1, Cas9, Cas3, Cas8a-c, CaslO, CasX, CasY, Cas13, Cas14, Cse1, Csy1, Csn2, Cas4, Csm2, Cm5 or a combination thereof.

83. The method of claim 80, wherein the CRISPR/Cas system protein comprises Cas9, Cpf1 or a combination thereof.

84. The method of claim 80, wherein CRISPR/Cas system protein is a Cas9 or Cpf1 nickase.

85. The method of claim 80, wherein CRISPR/Cas system protein is thermostable.

86. The method of claim 80, wherein the gNAs are deoxyribonucleic acids (gDNAs) or ribonucleic acids (gRNAs).

87. The method of claim 80, wherein the plurality of gNAs comprise at least 2, 10, 102, 103, 104, 105 or 106 unique gNAs.

88. The method of claim 80, comprising sequencing the library of nucleic acids.

89. The method of claim 88, wherein the sequencing is high throughput sequencing.

90. The method of claim 59, wherein the plurality of cDNAs comprise 3′ polyC sequences.


SEQUENCE LISTING (12 sequences)

SEQ ID NO: 1 - DNA, 19 bp, synthetic construct
  misc_feature 1..19: note = T7 promoter
  source 1..19: mol_type = other DNA
taatacgactcactatagg

SEQ ID NO: 2 - DNA, 20 bp, synthetic construct
  misc_feature 1..20: note = T7 promoter
  source 1..20: mol_type = other DNA
taatacgactcactataggg

SEQ ID NO: 3 - DNA, 29 bp, synthetic construct
  misc_feature 1..29: note = T7 promoter
  source 1..29: mol_type = other DNA
gcctcgagctaatacgactcactatagag

SEQ ID NO: 4 - DNA, 18 bp, synthetic construct
  misc_feature 1..18: note = SP6 promoter
  source 1..18: mol_type = other DNA
atttaggtgacactatag

SEQ ID NO: 5 - DNA, 24 bp, synthetic construct
  misc_feature 1..24: note = SP6 promoter
  source 1..24: mol_type = other DNA
catacgatttaggtgacactatag

SEQ ID NO: 6 - DNA, 18 bp, synthetic construct
  misc_feature 1..18: note = T3 promoter
  source 1..18: mol_type = other DNA
aattaaccctcactaaag

SEQ ID NO: 7 - RNA, 19 nt, synthetic construct
  misc_feature 1..19: note = Cpf1 guide RNA
  source 1..19: mol_type = other RNA
aatttctactgttgtagat

SEQ ID NO: 8 - DNA, 19 bp, synthetic construct
  misc_feature 1..19: note = Cpf1 guide RNA
  source 1..19: mol_type = other DNA
aatttctactgttgtagat

SEQ ID NO: 9 - DNA, 19 bp, synthetic construct
  misc_feature 1..19: note = Cpf1 guide RNA
  source 1..19: mol_type = other DNA
atctacaacagtagaaatt

SEQ ID NO: 10 - DNA, 83 bp, synthetic construct
  misc_feature 1..83: note = Cas9 single guide RNA
  source 1..83: mol_type = other DNA
gttttagagctagaaatagcaagttaaaataaggctagtccgttatcaacttgaaaaagtggcaccgagtcggtgcttttttt

SEQ ID NO: 11 - DNA, 83 bp, synthetic construct
  misc_feature 1..83: note = Cas9 single guide RNA
  source 1..83: mol_type = other DNA
aaaaaaagcaccgactcggtgccactttttcaagttgataacggactagccttattttaacttgctatttctagctctaaaac

SEQ ID NO: 12 - RNA, 83 nt, synthetic construct
  misc_feature 1..83: note = Cas9 single guide RNA
  source 1..83: mol_type = other RNA
gttttagagctagaaatagcaagttaaaataaggctagtccgttatcaacttgaaaaagtggcaccgagtcggtgcttttttt